(19) United States
(12) Patent Application Publication
(10) Pub. No.: US 2023/0039614 A1
(43) Pub. Date: Feb. 9, 2023

(54) MICROSIMULATION OF MULTI-CANCER EARLY DETECTION EFFECTS USING PARALLEL PROCESSING AND INTEGRATION OF FUTURE INTERCEPTED INCIDENCES OVER TIME

(71) Applicant: GRAIL, LLC, Menlo Park, CA (US)

(72) Inventors: Nan Zhang, Palo Alto, CA (US); Jing Zhang, Los Altos, CA (US); Earl Hubbell, Palo Alto, CA (US); Jerome Victor Braun, Davis, CA (US); Noah Robin Simon, Seattle, WA (US)

(21) Appl. No.: 17/879,777

(22) Filed: Aug. 3, 2022

Related U.S. Application Data
(60) Provisional application No. 63/230,464, filed on Aug. 6, 2021.

(51) Int. Cl. G16H 50/20; G16B 5/00; G16B 30/00; G16B 40/20

(52) U.S. Cl. CPC G16H 50/20; G16B 5/00; G16B 30/00; G16B 40/20

(57) ABSTRACT

A simulation system performs microsimulations to model the impact of one or more early cancer detection screenings for a plurality of participants to simulate a randomized controlled trial (RCT). In one instance, the microsimulations are performed using parallel processing techniques. The microsimulation simulates the impact of early detection screenings on individual trajectories of the participants. In particular, while most screening modalities are for single cancer types, the microsimulation herein simulates the effect of a detection model on individual trajectories for participant populations having multiple types of cancer using, for example, multi-cancer early detection (MCED) screenings that are capable of detecting multiple types of cancer.

### CROSS-REFERENCE TO RELATED APPLICATIONS

This application claims the benefit of U.S. Provisional Patent Application No. 62/230,464 filed on Aug. 6, 2021, which is hereby incorporated by reference in its entirety.

### BACKGROUND

Cancer screening aims to prevent cancer death by detecting cancerous lesions early, when treatment is still a viable option and more effective. However, with some exceptions, many cancers lack available screening modalities. The diagnosis of these cancers, often by the presentation of clinical symptoms, typically occurs in later stages when the cancer has already spread to other parts of the body and when the chances for survival are much lower. A multi-cancer early detection (MCED) test can be trained to generate detection estimates for multiple cancer types across stages. If the MCED test can detect cancers at earlier stages, then it can induce a paradigm shift in terms of when cancer can be treated. The clinical utility of the MCED test will be piloted in a large-scale randomized controlled trial (RCT), with the aim to assess stage shift and evaluate mortality reduction.

### SUMMARY

A method for simulating early cancer detection for a plurality of participants having multiple types of cancer are described. A cancer progression for each of a plurality of participants can be simulated using a simulation model. A first subset of the participants can be assigned to a control group and a second subset of the participants can be assigned to an intervention group. For each simulated participant in the plurality of participants, a cancer type and a diagnostic stage of cancer for the participant can be sampled. The participant can be expected to be clinically diagnosed with the cancer type at the diagnostic stage after a diagnosis time period for the participant has passed with respect to an enrollment time. A cancer trajectory for the participant can be generated that indicates progression of one or more stages of cancer for the participant and a survival time can be determined based on the cancer type and the diagnostic stage for the participant. A first detection time can be determined that indicates a time the cancer of the participant was correctly detected at a first detection stage equal to or earlier than the diagnostic stage. For example, in one embodiment, for participants assigned to a control group, the first detection time can be determined that indicates a time the cancer of the participant was correctly detected at a first detection stage equal to the diagnostic stage.

For each simulated participant in the second subset of participants of the intervention group, an early cancer detection screening can be simulated by one or more machine-learned cancer detection models for at least one screening time. Specifically, an indication for the participant can be generated that indicates whether the machine-learned cancer detection models predict that the participant has cancer. If the indication of the cancer detection models predict the participant has cancer and the participant has an earlier stage of cancer at the at least one screening time than the first detection stage, a second detection stage can be generated as the earlier stage and the survival time is updated based on the second detection stage. For each of the first subset of participants and the second subset of participants, one or more metrics related to progression of cancer can be evaluated. A difference between the one or more metrics of the first subset of participants and the second subset of participants can be evaluated to determine an effect of early cancer detection screening.

### BRIEF DESCRIPTION OF DRAWINGS

FIG. 1A is a flowchart describing a process of sequencing a fragment of cell-free (cf) DNA to obtain a methylation state vector, according to an embodiment.

FIG. 1B is an illustration of the process of FIG. 1A of sequencing a fragment of cell-free (cf) DNA to obtain a methylation state vector, according to an embodiment.

FIGS. 2A & 2B illustrate flowcharts describing a process of determining anomalously methylated fragments from a sample, according to an embodiment.

FIG. 3A is a flowchart describing a process of training a cancer classifier, according to an embodiment.

FIG. 3B illustrates an example generation of feature vectors used for training the cancer classifier, according to an embodiment.

FIG. 4A illustrates a flowchart of devices for sequencing nucleic acid samples according to one embodiment.

FIG. 4B is a block diagram of an analytics system, according to an embodiment.

FIG. 5 illustrates many graphs showing cancer prediction accuracy of a multiclass cancer classifier for various cancer types, according to an example implementation.

FIG. 6 illustrates many graphs showing cancer prediction accuracy of a multiclass cancer classifier for various cancer types after first using a binary cancer classifier, according to an example implementation.

FIG. 7 illustrates a confusion matrix demonstrating performance of a trained cancer classifier, according to an example implementation.

FIG. 8 illustrates a graph of cancer type likelihood for non-cancer samples above 95% specificity.

FIGS. 9A and 9B illustrate graphs of hematological subtypes separated according to methylation sequencing data.

FIG. 10A illustrates a flowchart describing a process of determining a binary threshold cutoff for binary cancer classification, in accordance with one or more embodiments.

FIG. 10B illustrates a flowchart describing a process of thresholding a TOO label for determining a binary threshold cutoff for binary cancer classification, in accordance with one or more embodiments.

FIG. 11 illustrates a confusion matrix demonstrating performance of a trained cancer tissue of origin classifier with additional hematological cancer subtypes.

FIGS. 12A and 12B illustrate graphs showing cancer prediction accuracy for cancer classifiers with and without adjusting a threshold cutoff for numerous cancer types over stages of cancer.

FIG. 13A illustrates a process for stratifying hematological signals into two strata, in accordance with one or more embodiments.

FIG. 13B illustrates a process for stratifying hematological signals into three strata, in accordance with one or more embodiments.

FIG. 13C illustrates a process for first stratifying hematological signals, and subsequently stratifying colorectal signals, in accordance with one or more embodiments.

FIG. 14 illustrates a process of determining binary threshold cutoffs for TOO stratification, in accordance with one or more embodiments.

FIG. 15 illustrates a flowchart describing a process of predicting cancer presence or cancer absence for a test sample using a binary threshold cutoff determined by TOO stratification, in accordance with one or more embodiments.

FIG. 16A illustrates a graph showing the classifier's sensitivity at 99.5% specificity level across the hematological subtypes.

FIG. 16B illustrates a graph showing the classifier's sensitivity at 95% specificity across stages for Hodgkin lymphomas and Non-Hodgkin lymphomas.

FIG. 17 illustrates a confusion matrix showing cancer prediction accuracy of the hematological-specific cancer classifier, in a first example implementation.

FIG. 18 illustrates a series of graphs plotting cancer score against distance from the centroid in the UMAP embedding for hematological-specific cancer classification, in the first example implementation.

FIG. 19 illustrates a graph plotting the anomaly scores of a plurality of training samples for hematological-specific cancer classification, in a second example implementation.

FIG. 20 illustrates a graph showing the hematological-specific cancer classifier's sensitivity at 99.5% specificity, in the second example implementation.

FIG. 21 illustrates a confusion matrix showing cancer prediction accuracy of the hematologic-specific cancer classifier, in the second example implementation.

FIG. 22 illustrates the simulated RCT structure and outcome measures at different study lengths, in accordance with one embodiment.

FIG. 23 illustrates simulation of a cancer trajectory for a participant from a control group or an intervention group, in accordance with one embodiment.

FIG. 24 illustrates simulation of a cancer trajectory for a participant from an intervention group with MCED screening, in accordance with one embodiment.

FIG. 25A are experimental results illustrating true positive, false positive, and positive predicted value (PPV) of an example RCT simulation. FIG. 25B are experimental results illustrating negative predicted value (NPV), sensitivity, specificity, cancer detection rate (CDR), CSO accuracy, and cancer incidence rate for simulated example MCED screening based on CCGA3 clinical study.

FIG. 26 are experimental results illustrating proportions of cancers detected in the intervention group compared to the control group after three rounds of MCED screening.

FIG. 27 are experimental results illustrating proportions of stage I and II cancers after three rounds of MCED screening.

FIG. 28 are experimental results illustrating absolute and relative reductions of stage III and IV cancers after three rounds of MCED screening.

FIG. 29 are experimental results illustrating mortality reduction under different types of tumor growth rates.

FIGS. 30A and 30B illustrates a flowchart describing a process of computer-simulating a RCT to determine impacts of MCED screenings, in accordance with an embodiment.

FIG. 31 illustrates an interception model approach that identifies different subpopulations of detectability, according to one embodiment.

FIGS. 32A-32C illustrate cancer incidence as a function of age as obtained from SEER data, according to one embodiment.

FIG. 33 illustrates a weight matrix for a MCED screening as a function of time from screen for a subpopulation that would be clinically diagnosed at stage IV but is first detectable at stage I for lung cancer, according to one embodiment.

FIG. 34 illustrates weight matrices for a MCED screening as a function of time from screen for one or more subpopulations, according to one embodiment.

FIG. 35 illustrates intercepted incidences of lung cancer as a function of age post screen for a particular cancer type and starting age, according to one embodiment.

FIG. 36 illustrates intercepted incidences of lung cancer by clinical diagnosis stage, according to one embodiment.

FIG. 37 illustrates intercepted incidences of lung cancer over all clinical diagnosis stages, according to one embodiment.

FIG. 38 illustrates intercepted incidences for each starting age, according to one embodiment.

FIG. 39 illustrates a comparison of interception incidences modeled by the integration system with interception incidences modeled by an interception model, according to one embodiment.

FIG. 40 illustrate a flowchart describing a process of an integrated method for determining intercepted incidences for MCED screenings, in accordance with an embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

### I.A. Overview of Methylation

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy individuals, may provide insight into a subject's cancer status. DNA methylation anomalies (compared to healthy controls) can cause different effects, which may contribute to cancer. Various challenges arise in the identification of anomalously methylated cfDNA fragments. First off, determining a DNA fragment to be anomalously methylated can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site can causally influence methylation at a subsequent CpG site. To encapsulate this dependency can be another challenge in itself.

Methylation can occur in deoxyribonucleic acid (DNA) when a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein are the same, and consequently the inventive concepts described herein are applicable to those other forms of methylation.

### I.B. Definitions

The term “individual” refers to a human individual. The term “healthy individual” refers to an individual presumed to not have a cancer or disease. The term “subject” refers to an individual who is known to have, or potentially has, a cancer or disease. Likewise, the term “subject” can also encompass non-human animals or any other living organism.

The term “cell free nucleic acid” or “cfNA” refers to nucleic acid fragments that circulate in an individual's body (e.g., blood) and originate from one or more healthy cells and/or from one or more cancer cells. The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “sequence read” refers to a nucleotide sequence obtained from a nucleic acid molecule from a test sample from an individual. Sequence reads can be obtained through various methods known in the art.

The term “sequencing depth” or “depth” refers to a total number of sequence reads or read segments at a given genomic location or loci from a test sample from an individual.

The term “anomalous fragment,” “anomalously methylated fragment,” or “fragment with an anomalous methylation pattern” refers to a fragment that has anomalous methylation of CpG sites. Anomalous methylation of a fragment may be determined using probabilistic models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “anomaly score” refers to a score for a CpG site based on a number of anomalous fragments (or, in some embodiments, UFXMs) from a sample overlaps that CpG site. The anomaly score is used in context of featurization of a sample for classification.

### II. Sample Processing

### II. A. Generating Methylation State Vectors for DNA Fragments

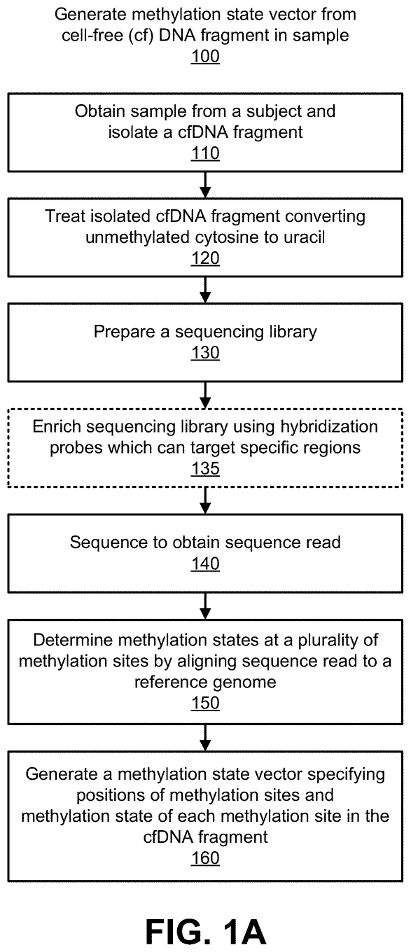

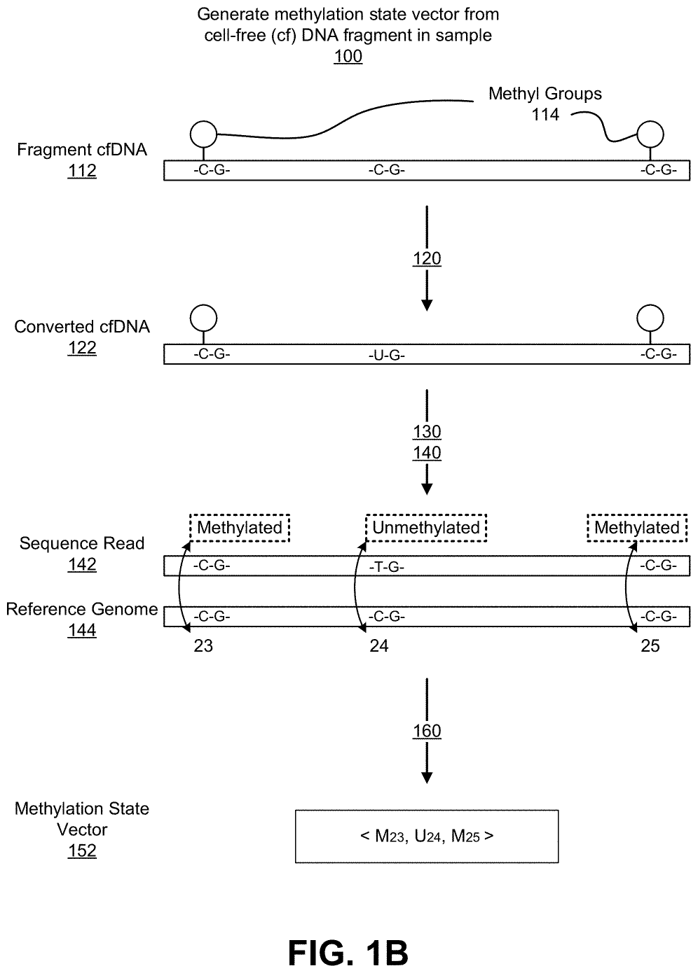

FIG. 1A is a flowchart describing a process 100 of sequencing a fragment of cell-free (cf) DNA to obtain a methylation state vector, according to an embodiment. In order to analyze DNA methylation, an analytics system first obtains 110 a sample from an individual comprising a plurality of cfDNA molecules. Generally, samples may be from healthy individuals, subjects known to have or suspected of having cancer, or subjects where no prior information is known. The test sample may be a sample selected from the group consisting of blood, plasma, serum, urine, fecal, and saliva samples. Alternatively, the test sample may comprise a sample selected from the group consisting of whole blood, a blood fraction (e.g., white blood cells (WBCs)), a tissue biopsy, pleural fluid, pericardial fluid, cerebral spinal fluid, and peritoneal fluid. In additional embodiments, the process 100 may be applied to sequence other types of DNA molecules.

From the sample, the analytics system can isolate each cfDNA molecule. The cfDNA molecules can be treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cfDNA molecules, a sequencing library is prepared 130. Optionally, the sequencing library may be enriched 135 for cfDNA molecules, or genomic regions, that are informative for cancer status using a plurality of hybridization probes. The hybridization probes can be short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. In one embodiment, the hybridization probes are designed to enrich for DNA molecules that have been treated (e.g., using bisulfite) for conversion of unmethylated cytosines to uracils. Once prepared, the sequencing library or a portion thereof can be sequenced to obtain a plurality of sequence reads. The sequence reads may be in a computer-readable, digital format for processing and interpretation by computer software. The number of sequence reads may be at least 1, 10, 1000, 5000, 10000, 20000, 50000, or more. In some embodiments, the number of sequence read may be at most 100000, 50000, 20000, 10000, 5000, 1000, 20 or less.

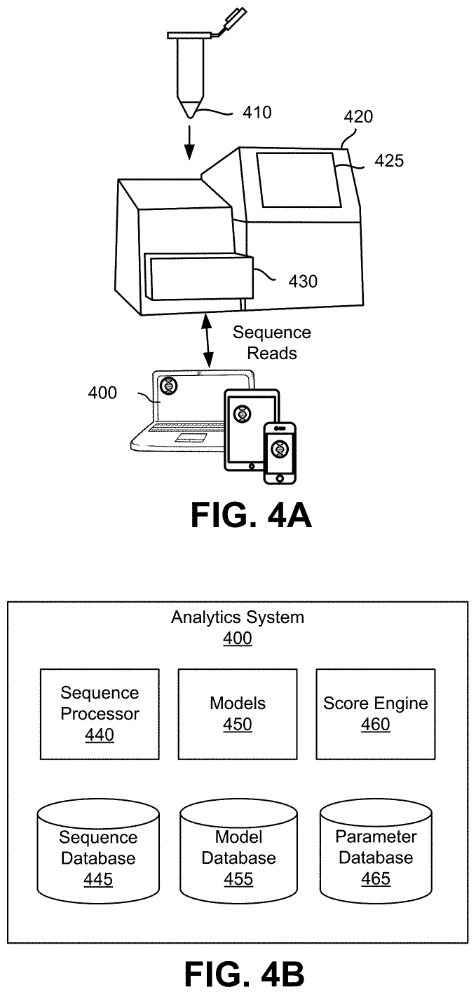

From the sequence reads, the analytics system determines 150 a location and methylation state for each CpG site based on alignment to a reference genome. The analytics system generates 160 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states can be states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses; one such model will be described below in conjunction with FIG. 4.

FIG. 1B is an illustration of the process 100 of FIG. 1A of sequencing a cfDNA molecule to obtain a methylation state vector, according to an embodiment. As an example, the analytics system receives a cfDNA molecule 112 that, in this example, contains three CpG sites. As shown, the first and third CpG sites of the cfDNA molecule 112 are methylated 114. During the treatment step 120, the cfDNA molecule 112 is converted to generate a converted cfDNA molecule 122. During the treatment 120, the second CpG site which was unmethylated has its cytosine converted to uracil. However, the first and third CpG sites were not converted.

After conversion, a sequencing library 130 is prepared and sequenced 140 generating a sequence read 142. The analytics system aligns 150 the sequence read 142 to a reference genome 144. The reference genome 144 provides the context as to what position in a human genome the fragment cfDNA originates from. In this simplified example, the analytics system aligns 150 the sequence read 142 such that the three CpG sites correlate to CpG sites 23, 24, and 25 (arbitrary reference identifiers used for convenience of description). The analytics system thus generates information both on methylation status of all CpG sites on the cfDNA molecule 112 and the position in the human genome that the CpG sites map to. As shown, the CpG sites on sequence read 142 which were methylated are read as cytosines. In this example, the cytosines appear in the sequence read 142 in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system generates 160 a methylation state vector 152 for the fragment cfDNA 112. In this example, the resulting methylation state vector 152 is <M₂₃, U₂₄, M₂₅>, wherein M corresponds to a methylated CpG site, U corresponds to an unmethylated CpG site, and the subscript number corresponds to a position of each CpG site in the reference genome.

### II.B. Identifying Anomalous Fragments

The analytics system can determine anomalous fragments for a sample using the sample's methylation state vectors. For each fragment in a sample, the analytics system can determine whether the fragment is an anomalous fragment using the methylation state vector corresponding to the fragment. In one embodiment, the analytics system calculates a p-value score for each methylation state vector describing a probability of observing that methylation state vector or other methylation state vectors even less probable in the healthy control group. The process for calculating a p-value score will be further discussed below in Section II.B.i. P-Value Filtering. The analytics system may determine fragments with a methylation state vector having below a threshold p-value score as anomalous fragments. In another embodiment, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

### II.B.I P-Value Filtering

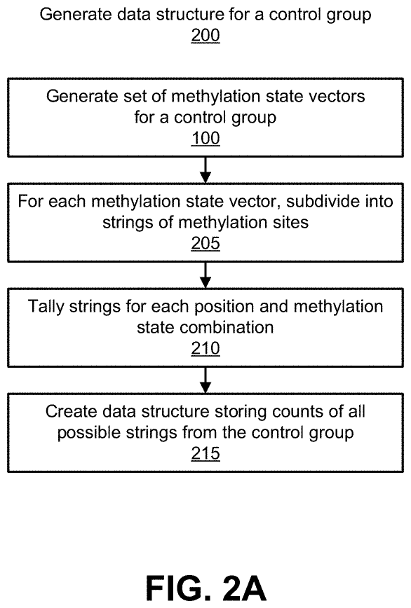

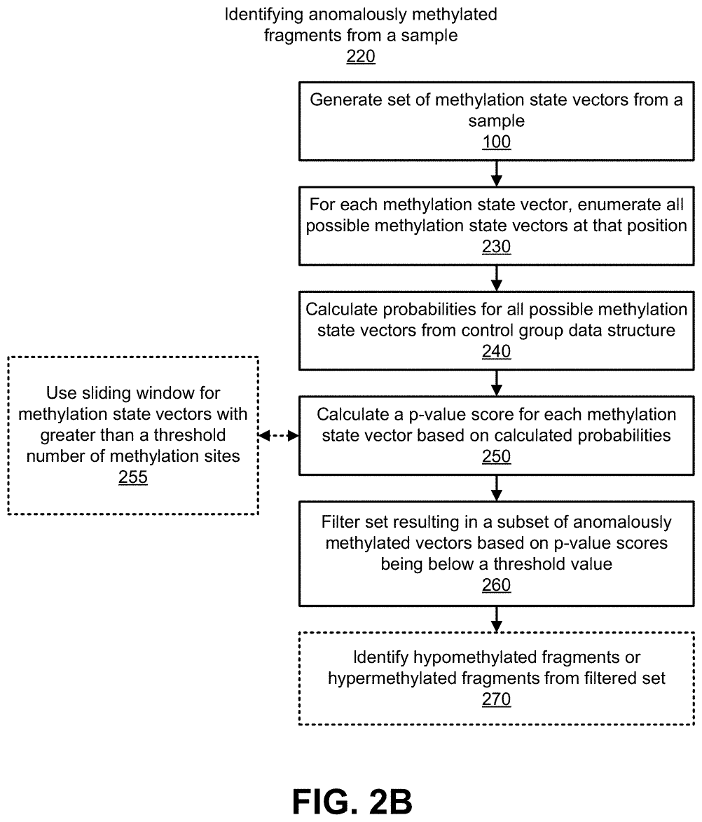

In one embodiment, the analytics system calculates a p-value score for each methylation state vector compared to methylation state vectors from fragments in a healthy control group. The p-value score can describe a probability of observing the methylation status matching that methylation state vector or other methylation state vectors even less probable in the healthy control group. In order to determine a DNA fragment to be anomalously methylated, the analytics system can use a healthy control group with a majority of fragments that are normally methylated. When conducting this probabilistic analysis for determining anomalous fragments, the determination can hold weight in comparison with the group of control subjects that make up the healthy control group. To ensure robustness in the healthy control group, the analytics system may select some threshold number of healthy individuals to source samples including DNA fragments. FIG. 2A below describes the method of generating a data structure for a healthy control group with which the analytics system may calculate p-value scores. FIG. 2B describes the method of calculating a p-value score with the generated data structure.

FIG. 2A is a flowchart describing a process 200 of generating a data structure for a healthy control group, according to an embodiment. To create a healthy control group data structure, the analytics system can receive a plurality of DNA fragments (e.g., cfDNA) from a plurality of healthy individuals. A methylation state vector can be identified for each fragment, for example via the process 100.

With each fragment's methylation state vector, the analytics system subdivides 205 the methylation state vector into strings of CpG sites. In one embodiment, the analytics system subdivides 205 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 210 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 210 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 215 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory used) compared to the prior string length. Reducing string size can help keep the data structure creation and performance (e.g., use for later accessing as described below), in terms of computational and storage, reasonable. Second, a statistical consideration to limiting the maximum string length can avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it uses a significant amount of data that may not be available, and thus would be too sparse for a model to perform appropriately. For example, calculating a probability of anomalousness/cancer conditioned on the prior 100 CpG sites may use counts of strings in the data structure of length 100, ideally some matching exactly the prior 100 methylation states. If sparse counts of strings of length 100 are available, there may be insufficient data to determine whether a given string of length of 100 in a test sample is anomalous or not.

FIG. 2B is a flowchart describing a process 220 for identifying anomalously methylated fragments from an individual, according to an embodiment. In process 220, the analytics system generates 100 methylation state vectors from cfDNA fragments of the subject. The analytics system handles each methylation state vector as follows.

For a given methylation state vector, the analytics system enumerates 230 all possibilities of methylation state vectors having the same starting CpG site and same length (i.e., set of CpG sites) in the methylation state vector. As each methylation state is generally either methylated or unmethylated, there can be effectively two possible states at each CpG site, and thus the count of distinct possibilities of methylation state vectors can depend on a power of 2, such that a methylation state vector of length n can be associated with 2ⁿ possibilities of methylation state vectors. With methylation state vectors inclusive of indeterminate states for one or more CpG sites, the analytics system may enumerate 230 possibilities of methylation state vectors considering CpG sites that have observed states.

The analytics system calculates 240 the probability of observing each possibility of methylation state vector for the identified starting CpG site and methylation state vector length by accessing the healthy control group data structure. In one embodiment, calculating the probability of observing a given possibility uses a Markov chain probability to model the joint probability calculation. In other embodiments, calculation methods other than Markov chain probabilities are used to determine the probability of observing each possibility of methylation state vector.

The analytics system calculates 250 a p-value score for the methylation state vector using the calculated probabilities for each possibility. In one embodiment, this includes identifying the calculated probability corresponding to the possibility that matches the methylation state vector in question. Specifically, this is the possibility having the same set of CpG sites, or similarly the same starting CpG site and length as the methylation state vector. The analytics system can sum the calculated probabilities of any possibilities having probabilities less than or equal to the identified probability to generate the p-value score.

This p-value can represent the probability of observing the methylation state vector of the fragment or other methylation state vectors even less probable in the healthy control group. A low p-value score, thereby, generally corresponds to a methylation state vector which is rare in a healthy individual, and which causes the fragment to be labeled anomalously methylated, relative to the healthy control group. A high p-value score generally relates to a methylation state vector is expected to be present, in a relative sense, in a healthy individual. If the healthy control group is a non-cancerous group, for example, a low p-value can indicate that the fragment is anomalous methylated relative to the non-cancer group, and therefore possibly indicative of the presence of cancer in the test subject.

As above, the analytics system can calculate p-value scores for each of a plurality of methylation state vectors, each representing a cfDNA fragment in the test sample. To identify which of the fragments are anomalously methylated, the analytics system may filter 260 the set of methylation state vectors based on their p-value scores. In one embodiment, filtering is performed by comparing the p-values scores against a threshold and keeping those fragments below the threshold. This threshold p-value score can be on the order of 0.1, 0.01, 0.001, 0.0001, or similar.

According to example results from the process 400, the analytics system yields a median (range) of 2,800 (1,500-12,000) fragments with anomalous methylation patterns for participants without cancer in training, and a median (range) of 3,000 (1,200-220,000) fragments with anomalous methylation patterns for participants with cancer in training. These filtered sets of fragments with anomalous methylation patterns may be used for the downstream analyses as described below in Section III.

In one embodiment, the analytics system uses 255 a sliding window to determine possibilities of methylation state vectors and calculate p-values. Rather than enumerating possibilities and calculating p-values for entire methylation state vectors, the analytics system can enumerate possibilities and calculates p-values for a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window can identify the sequential set of CpG sites from the vector within the window starting from the first CpG site in the vector. The analytic system can calculate a p-value score for the window including the first CpG site. The analytics system can then “slide” the window to the second CpG site in the vector, and calculate another p-value score for the second window. Thus, for a window size/and methylation vector length m, each methylation state vector can generate m−/+1 p-value scores. After completing the p-value calculations for each portion of the vector, the lowest p-value score from all sliding windows can be taken as the overall p-value score for the methylation state vector. In another embodiment, the analytics system aggregates the p-value scores for the methylation state vectors to generate an overall p-value score.

Using the sliding window can help to reduce the number of enumerated possibilities of methylation state vectors and their corresponding probability calculations that would otherwise be performed. To give a realistic example, it is possible for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations can enumerate 2\hat{ }5 (32) possibilities of methylation state vectors, which total results in 50×2\hat{ }5 (1.6×10\hat{ }3) probability calculations. This can result in a vast reduction of calculations to be performed, with no meaningful hit to the accurate identification of anomalous fragments.

In embodiments with indeterminate states, the analytics system may calculate a p-value score summing out CpG sites with indeterminates states in a fragment's methylation state vector. The analytics system can identify all possibilities that have consensus with the all methylation states of the methylation state vector excluding the indeterminate states. The analytics system may assign the probability to the methylation state vector as a sum of the probabilities of the identified possibilities. As an example, the analytics system calculates a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states can use calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm can operate in linear computational time.

In one embodiment, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities can allow for efficient calculation of p-score values without re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

### II.B.II. Hypermethylated Fragments and Hypomethylated Fragments

In another embodiment, the analytics system determines anomalous fragments as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### II.C. Example Analytics System

FIG. 4A is a flowchart of devices for sequencing nucleic acid samples according to one embodiment. This illustrative flowchart includes devices such as a sequencer 420 and an analytics system 400. The sequencer 420 and the analytics system 400 may work in tandem to perform one or more steps in the processes 100 of FIG. 1A, 200 of FIG. 2A, 220 of FIG. 2B, and other process described herein.

In various embodiments, the sequencer 420 receives an enriched nucleic acid sample 410. As shown in FIG. 4A, the sequencer 420 can include a graphical user interface 425 that enables user interactions with particular tasks (e.g., initiate sequencing or terminate sequencing) as well as one more loading stations 430 for loading a sequencing cartridge including the enriched fragment samples and/or for loading buffers for performing the sequencing assays. Therefore, once a user of the sequencer 420 has provided the reagents and sequencing cartridge to the loading station 430 of the sequencer 420, the user can initiate sequencing by interacting with the graphical user interface 425 of the sequencer 420. Once initiated, the sequencer 420 performs the sequencing and outputs the sequence reads of the enriched fragments from the nucleic acid sample 410.

In some embodiments, the sequencer 420 is communicatively coupled with the analytics system 400. The analytics system 400 includes some number of computing devices used for processing the sequence reads for various applications such as assessing methylation status at one or more CpG sites, variant calling or quality control. The sequencer 420 may provide the sequence reads in a BAM file format to the analytics system 400. The analytics system 400 can be communicatively coupled to the sequencer 420 through a wireless, wired, or a combination of wireless and wired communication technologies. Generally, the analytics system 400 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information, e.g., via step 140 of the process 100 in FIG. 1A. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 400 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. An output file having SAM (sequence alignment map) format or BAM (binary) format may be generated and output for further analysis.

Referring now to FIG. 4B, FIG. 4B is a block diagram of an analytics system 400 for processing DNA samples according to one embodiment. The analytics system implements one or more computing devices for use in analyzing DNA samples. The analytics system 400 includes a sequence processor 440, sequence database 445, model database 455, models 450, parameter database 465, and score engine 460. In some embodiments, the analytics system 400 performs some or all of the processes 100 of FIG. 1A and 200 of FIG. 2.

The sequence processor 440 generates methylation state vectors for fragments from a sample. At each CpG site on a fragment, the sequence processor 440 generates a methylation state vector for each fragment specifying a location of the fragment in the reference genome, a number of CpG sites in the fragment, and the methylation state of each CpG site in the fragment whether methylated, unmethylated, or indeterminate via the process 100 of FIG. 1A. The sequence processor 440 may store methylation state vectors for fragments in the sequence database 445. Data in the sequence database 445 may be organized such that the methylation state vectors from a sample are associated to one another.

Further, multiple different models 450 may be stored in the model database 455 or retrieved for use with test samples. In one example, a model is a trained cancer classifier for determining a cancer prediction for a test sample using a feature vector derived from anomalous fragments. The training and use of the cancer classifier will be further discussed in conjunction with Section III. Cancer Classifier for Determining Cancer. The analytics system 400 may train the one or more models 450 and store various trained parameters in the parameter database 465. The analytics system 400 stores the models 450 along with functions in the model database 455.

During inference, the score engine 460 uses the one or more models 450 to return outputs. The score engine 460 accesses the models 450 in the model database 455 along with trained parameters from the parameter database 465. According to each model, the score engine receives an appropriate input for the model and calculates an output based on the received input, the parameters, and a function of each model relating the input and the output. In some use cases, the score engine 460 further calculates metrics correlating to a confidence in the calculated outputs from the model. In other use cases, the score engine 460 calculates other intermediary values for use in the model.

### III. Cancer Classifier for Determining Cancer

### III. A. Overview

The cancer classifier can be trained to receive a feature vector for a test sample and determine whether the test sample is from a test subject that has cancer or, more specifically, a particular cancer type. The cancer classifier can comprise a plurality of classification parameters and a function representing a relation between the feature vector as input and the cancer prediction as output determined by the function operating on the input feature vector with the classification parameters. In one embodiment, the feature vectors input into the cancer classifier are based on set of anomalous fragments determined from the test sample. The anomalous fragments may be determined via the process 220 in FIG. 2B, or more specifically hypermethylated and hypomethylated fragments as determined via the step 270 of the process 220, or anomalous fragments determined according to some other process. Prior to deployment of the cancer classifier, the analytics system trains the cancer classifier with the process 300.

### III.B. Training of Cancer Classifier

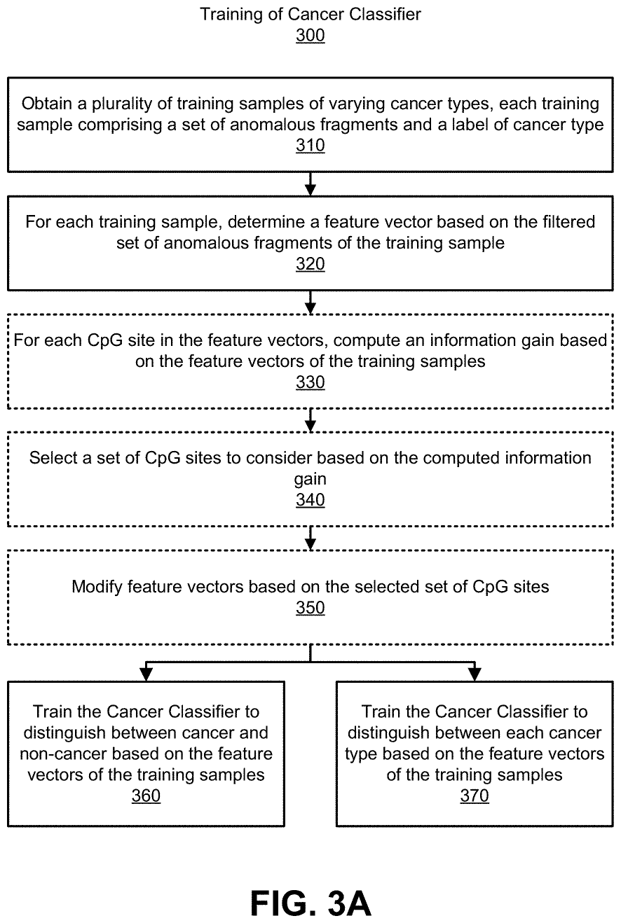

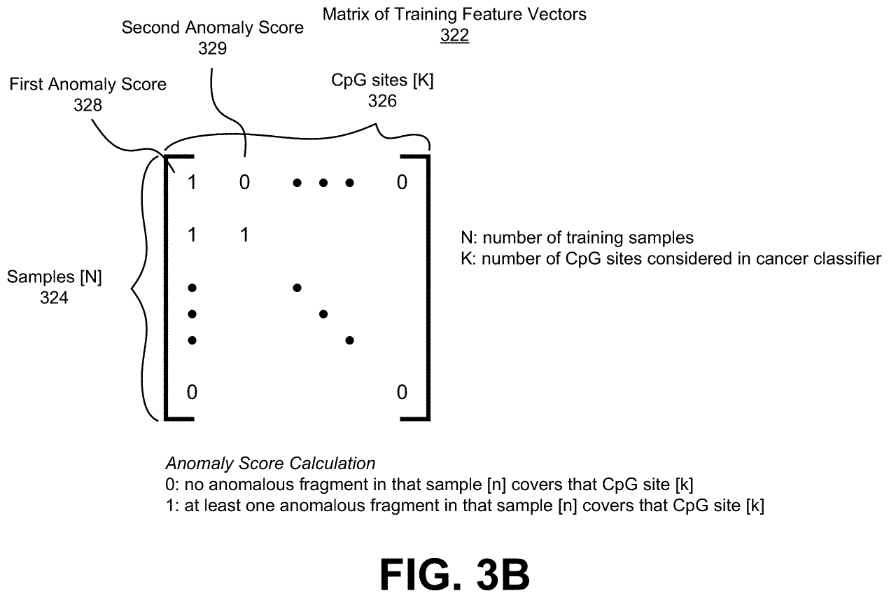

FIG. 3A is a flowchart describing a process 300 of training a cancer classifier, according to an embodiment. The analytics system obtains 310 a plurality of training samples each having a set of anomalous fragments and a label of a cancer type. The plurality of training samples includes any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 320, for each training sample, a feature vector based on the set of anomalous fragments of the training sample. The analytics system can calculate an anomaly score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragment in a sample that overlap the CpG site and calculates an anomaly score based on the count of 5.

Once all anomaly scores are determined for a training sample, the analytics system can determine the feature vector as a vector of elements including, for each element, one of the anomaly scores associated with one of the CpG sites in an initial set. The analytics system can normalize the anomaly scores of the feature vector based on a coverage of the sample. Here, coverage refers to a median or average sequencing depth over all CpG sites covered by the initial set of CpG sites used in the classifier, or based on the set of anomalous fragments for a given training sample.

As an example, reference is now made to FIG. 3B illustrating a matrix of training feature vectors 322. In this example, the analytics system has identified CpG sites [K] 326 for consideration in generating feature vectors for the cancer classifier. The analytics system selects training samples [N] 324. The analytics system determines a first anomaly score 328 for a first arbitrary CpG site [k1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 328 for the first CpG site as 1, as illustrated in FIG. 3B. Considering a second arbitrary CpG site [k2], the analytics system similarly checks the set of anomalous fragments for at least one that includes the second CpG site [k2]. If the analytics system does not find any such anomalous fragment that includes the second CpG site, the analytics system determines a second anomaly score 329 for the second CpG site [k2] to be 0, as illustrated in FIG. 3B. Once the analytics system determines all the anomaly scores for the initial set of CpG sites, the analytics system determines the feature vector for the first training sample [n1] including the anomaly scores with the feature vector including the first anomaly score 328 of 1 for the first CpG site [k1] and the second anomaly score 329 of 0 for the second CpG site [k2] and subsequent anomaly scores, thus forming a feature vector [1, 0, . . . ].

The analytics system may further limit the CpG sites considered for use in the cancer classifier. The analytics system computes 330, for each CpG site in the initial set of CpG sites, an information gain based on the feature vectors of the training samples. From step 320, each training sample has a feature vector that may contain an anomaly score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 330 an information gain for each cancer type and for each CpG site in the initial set to determine whether to include that CpG site in the classifier. The information gain can be computed for training samples with a given cancer type compared to all other samples. For example, two random variables ‘anomalous fragment’ (‘AF’) and ‘cancer type’ (CT′) are used. In one embodiment, AF is a binary variable indicating whether there is an anomalous fragment overlapping a given CpG site in a given samples as determined for the anomaly score/feature vector above. CT can be a random variable indicating whether the cancer is of a particular type. The analytics system can compute the mutual information with respect to CT given AF. That can be, how many bits of information about the cancer type are gained if it is known whether there is an anomalous fragment overlapping a particular CpG site. In practice, for a first cancer type, the analytics system can compute pairwise mutual information gain against each other cancer type and sums the mutual information gain across all the other cancer types.

For a given cancer type, the analytics system can use this information to rank CpG sites based on how cancer specific they are. This procedure can be repeated for all cancer types under consideration. If a particular region is commonly anomalously methylated in training samples of a given cancer but not in training samples of other cancer types or in healthy training samples, then CpG sites overlapped by those anomalous fragments can have high information gains for the given cancer type. The ranked CpG sites for each cancer type can be greedily added (selected) 340 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 350 the feature vectors of the training samples. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 320 or to the selected set of CpG sites from step 350. In one embodiment, the analytics system trains 360 a binary cancer classifier to distinguish between cancer and non-cancer based on the feature vectors of the training samples. In this manner, the analytics system uses training samples that include both non-cancer samples from healthy individuals and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 450 a multiclass cancer classifier to distinguish between many cancer types (also referred to as tissue of origin (TOO) labels). Cancer types can include one or more cancers and may include a non-cancer type (may also include any additional other diseases or genetic disorders, etc.). To do so, the analytics system can use the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

### III.C. Tuning of Cancer Classifier

During use of the cancer classifier, the analytics system may perform operations to tune the predictive capabilities of the cancer classifier. Generally, a sample distribution may include one or more non-cancer samples with high tissue signal. Some of these high tissue signal non-cancer samples may even be pre-stage cancer, early stage cancer, or undiagnosed cancer. As such, non-cancer samples with high-tissue signal may muddle the predictive capabilities of the cancer classifier.

As used herein, “high tissue signal” refers to a sample with a tissue signal, e.g., generally for any type of tissue or for a particular cancer type—also referred to as a TOO label, that exceeds some threshold. The tissue signal may be determined by a multiclass cancer classifier or other approaches, in comparison to a healthy distribution. Non-cancer samples with high tissue signal can be outliers in the non-cancer distribution. Some of these non-cancer samples may be pre-stage cancer, early stage cancer, or undiagnosed cancer. The analytics system can identify non-cancer samples with high tissue signal in at least one TOO label. In one approach of determining high tissue signal, a prediction value for a TOO label output by the multiclass cancer classifier is compared against a tissue signal threshold. Samples with a prediction value above the tissue signal threshold can be deemed to have high tissue signal for that TOO label; whereas, samples with a prediction value below the tissue signal threshold can be deemed to not have high tissue signal for that TOO label (or low tissue signal). In another approach, one or more top predictions in a TOO prediction are considered. For example, a TOO prediction for a sample has a first prediction of the colorectal TOO label, a second prediction of the breast TOO label, and a third prediction of head/neck TOO label. If the top prediction is considered, then the sample can be deemed to have high tissue signal for the TOO label in the first prediction, that being the colorectal TOO label in the example. If the top two predictions are considered, then there can be high tissue signal in both the colorectal TOO label and the breast TOO label. Other approaches of determining tissue signal may include other models trained to determine tissue signal for one or more TOO labels. Such models may include classifiers trained to determine tissue signal for a subset of TOO labels. For example, a hematological-specific classifier may be trained and used to determine tissue signal for one or more hematological subtypes. Two such example implementations are described under Section V. Example Results of Cancer Classifier. Other models include deconvolution models that can deconvolve tissue signal from methylation sequencing data (and/or other types of sequencing data).

In one embodiment of tuning the classifier, excluding high tissue signal non-cancer samples improves the classifier's sensitivity in cancer prediction. With a pruned sample distribution, a binary threshold cutoff may be determined according to a minimum specificity, wherein the binary threshold cutoff is used to predict presence or absence of cancer in a test sample. This method is further elaborated under Section III.C.i. Removal of High Signal Non-Cancer Samples.

In another embodiment of tuning the cancer classifier, the sample distribution may be stratified according to TOO signal. The analytics system can determine a binary threshold cutoff for each resulting stratum with the samples stratified into the stratum. With a test sample, the analytics system can place the test sample into a stratum according to the TOO signal and predicts the presence or absence of cancer in the test sample with the stratum's binary threshold cutoff. This method can be further elaborated under Section III.C.ii. Stratification of Sample Distribution According to TOO Signal.

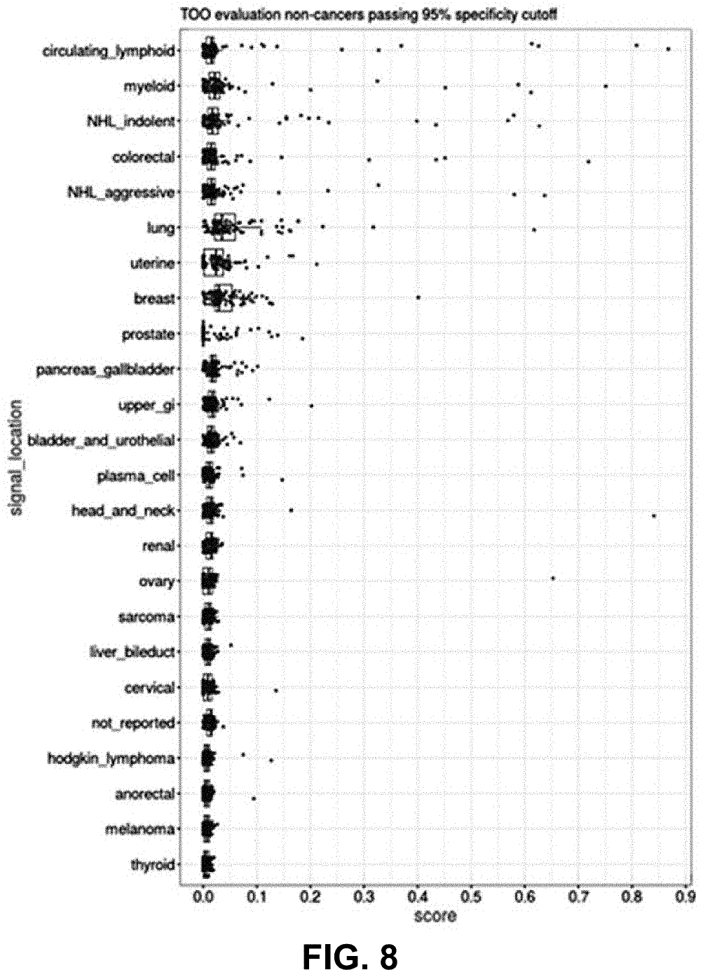

Referring to FIG. 8, FIG. 8 illustrates a graph of cancer type likelihood for non-cancer samples above 95% specificity. A cancer score was calculated for each non-cancer sample from a plurality of non-cancer samples, i.e., samples from healthy individuals not currently diagnosed with cancer. The cancer score can be determined by the binary classifier as a likelihood that a sample has cancer given the sample's methylation sequencing data. In other embodiments, the cancer score can be calculated according to other methods that input at least sequencing data (e.g., methylation, single nucleotide polymorphism (SNP), DNA, RNA, etc.) and output a sample's likelihood of having cancer based on the input sequencing data. One example of a classifier is a mixture model classifier. A distribution of the non-cancer samples can be generated according to the cancer scores of the non-cancer samples. A binary threshold cutoff can be set to ensure some level of binary classification specificity, e.g., a true negative rate. Typically, a high specificity cutoff can be used in classifying cancer, e.g., 99.4% specificity or higher. However, many non-cancer samples, used in training the cancer classifier and just below the specificity cutoff, can have high tissue signal thereby positively biasing the binary threshold cutoff.

In some embodiments, certain cancer types are further separated into cancer subtypes. For example, the hematological cancer type can further be separated into a combination of, for instance, circulating lymphoid subtype, non-Hodgkin's-Lymphoma (NHL) indolent subtype, NHL aggressive subtype, Hodgkin's-Lymphoma (HL) subtype, myeloid subtype, and plasma cell subtype, all of which also belong to a lymphoid neoplasm class.

To demonstrate, non-cancer samples above the 95% specificity were selected and then input into a multiclass cancer classifier to determine a probability for each cancer type—or tissue of origin (TOO). The cancer types or TOO labels used in this embodiment of the multiclass cancer classifier include circulating lymphoid, myeloid, NHL indolent, colorectal, NHL aggressive, lung, uterine, breast, prostate, pancreas and gallbladder, upper gastrointestinal, bladder and urothelial, plasma cell, head and neck, renal, ovary, sarcoma, liver and bile duct, cervical, other tissues, HL, anorectal, melanoma, thyroid. The graph in FIG. 8 shows many non-cancer samples having high tissue signal from at least one tissue type. Each dot in a row for a tissue type corresponds to a tissue of origin likelihood for a non-cancer sample above the 95% specificity threshold. Notably, many tissue types have multiple non-cancer sample outliers having significant tissue contribution, not typical for non-cancer samples. This can arise when such non-cancer samples have cfDNA signals being driven by cancer-like methylation, clonal fraction, and/or rate of growth/turnover. Nonetheless, these non-cancer samples with significant tissue contribution shift the binary classification threshold cutoff up thereby decreasing sensitivity of the cancer classification, especially with samples with significant tissue signal just below the previously set binary classification threshold cutoff. In practice, such signals (e.g., corresponding to circulating lymphoid, myeloid, and NHL indolent) can be a major attractor of false positive determinations. Of note, circulating lymphoid, myeloid, NHL indolent, colorectal, NHL aggressive, lung, uterine, breast, prostate, pancreas and gallbladder, upper gastrointestinal, plasma cell, head and neck, cervical, HL had at least one non-cancer sample with a probability of tissue origin above 0.1. Particularly, circulating lymphoid, myeloid, NHL indolent, and NHL aggressive (all hematological subtypes) had two or more non-cancer samples with a probability of tissue origin above 0.5.

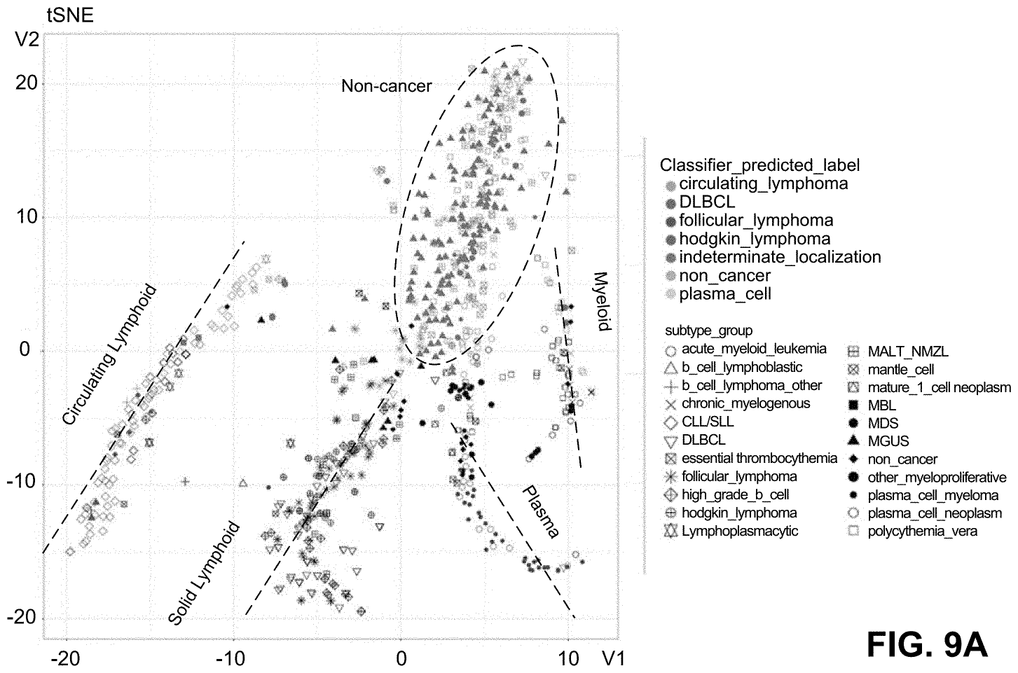

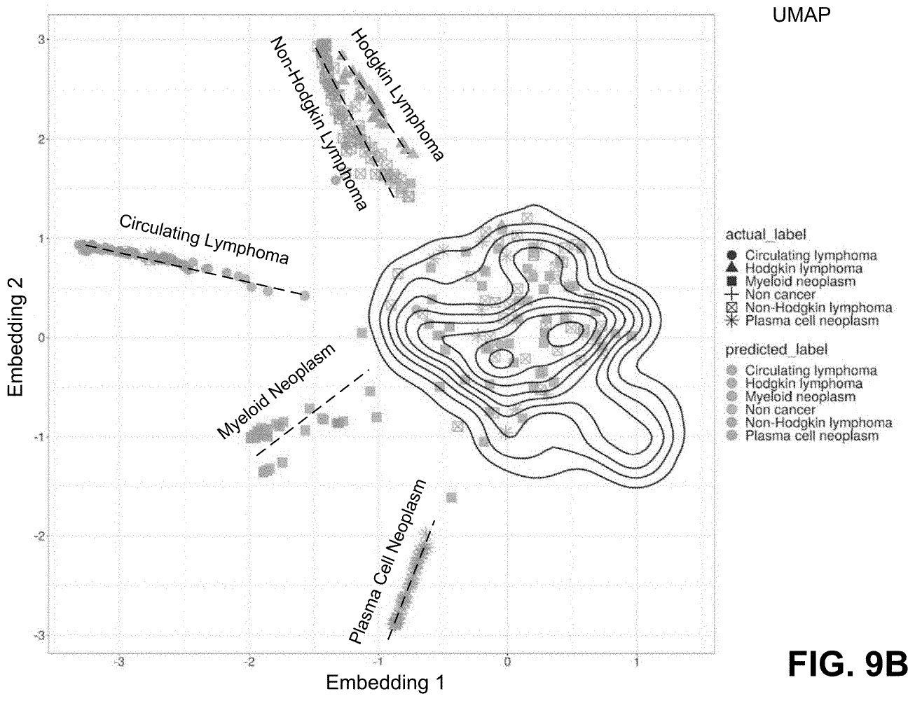

Referring to FIGS. 9A and 9B, FIGS. 9A and 9B illustrate graphs of hematological subtypes separated according to methylation sequencing data. The graphs of FIGS. 9A and 9B demonstrate an ability to model hematological subtypes. This can prove beneficial in providing more granularity to the multiclass cancer classification (e.g., classifying additionally with the hematological subtype labels) or as a manner of tuning the cancer classification through pruning non-cancer samples with high hematological subtype signal prior to training the cancer classifier. As described above, methylation signal can cover a plurality of CpG sites, thereby creating a high-dimensional vector space. The hematological subtypes shown include circulating lymphoid, solid lymphoid, plasma cell, and myeloid. The solid lymphoid subtype can be further divided into HL, NHL indolent, and NHL aggressive.

In FIG. 9A, the analytics system performs a t-distributed stochastic neighbor embedding. The t-distributed stochastic neighbor embedding identifies reduced dimensionality of the vector space (encompassing the methylation sequencing data) into a smaller number of embeddings. The embeddings are in order of variance in methylation signal amongst the samples. The first principal embedding, shown as “V1” on the horizontal axis on the graph, has the highest variance. The third principal embedding, shown as “V2” on the vertical axis on the graph, has the third highest variance. Annotated on the graph are clusters of the samples for each hematological subtype and non-cancer.

In FIG. 9B, the analytics system performs a UMAP embedding. The UMAP embedding also reduces dimensionality of the vector space into a smaller number of embeddings. The embeddings are in order of variance in methylation signal amongst the samples. The first principal embedding, shown as “embedding 1” on the horizontal axis on the graph, has the highest variance. The second principal embedding, shown as “embedding 2” on the vertical axis on the graph, has the second highest variance. Non-cancer samples are shown using a contour density.

The graphs show potential for classifying according to the hematological subtypes—either for addition of the hematological subtypes in the multiclass cancer classification or for modeling each of the hematological subtypes for tuning of the cancer classifiers.

### III.C.I. Removal of High Signal Non-Cancer Samples

In one or more embodiments, the analytics system tunes the trained cancer classifier by pruning the non-cancer samples used in training the cancer classifier. In particular, the analytics system may seek to remove non-cancer samples with high tissue signal that dilute the cancer classifier's sensitivity in cancer prediction.

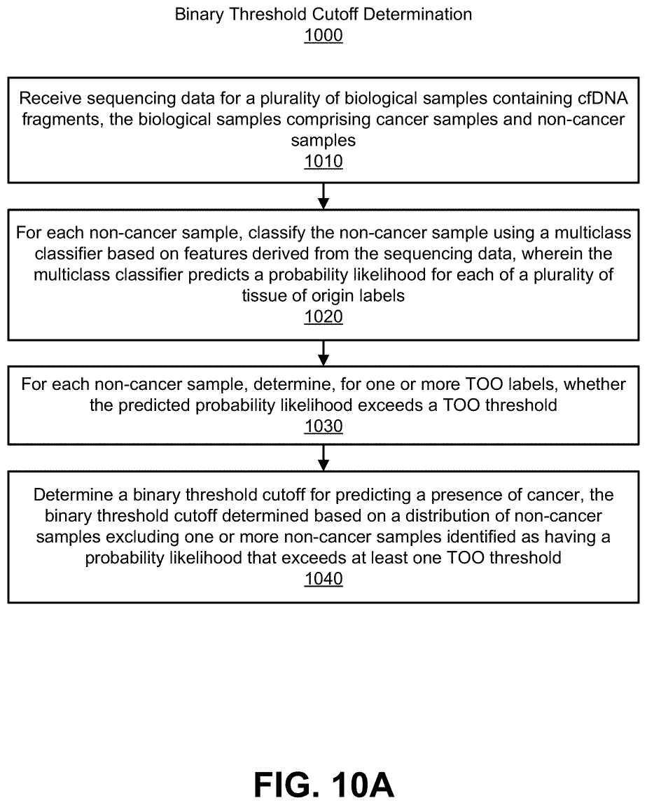

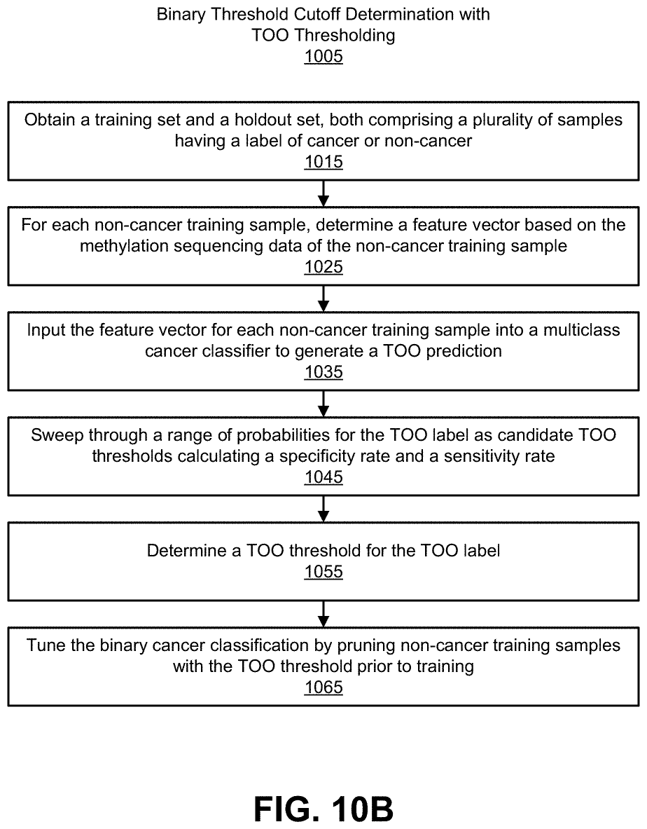

FIG. 10A illustrates a flowchart describing a process 1000 of determining a binary threshold cutoff for binary cancer classification, in accordance with one or more embodiments. A binary classification for predicting between cancer and non-cancer can evaluate a sample's cancer score against a determined binary threshold cutoff, wherein a sample with a cancer score below the binary threshold cutoff is determined to be non-cancer and with a cancer score at or above the binary threshold cutoff is determined to be cancer. A trained multiclass cancer classifier can evaluate a sample's methylation signal (and/or other sequencing data) to determine probabilities for a number of TOO labels classified by the multiclass cancer classifier. A TOO label used in a multiclass cancer classifier can be a cancer tissue type or a cancer tissue subtype (e.g., the hematological subtypes described above). The process 1000 can be performed or accomplished by the analytics system.

The analytics system receives 1010 sequencing data for a plurality of biological samples containing cfDNA fragments, the biological samples comprising cancer samples and non-cancer samples. The sequencing data can be methylation sequencing data, SNP sequencing data, another DNA sequencing data, RNA sequencing data, etc.

For each non-cancer sample, the analytics system classifies 1020 the non-cancer sample using a multiclass cancer classifier based on features derived from the sequencing, wherein the multiclass cancer classifier predicts a probability for each of a plurality of TOO labels. The analytics system can generate a feature vector for the non-cancer sample according to step 320 of FIG. 3A, i.e., assigning an anomaly score for each CpG site in consideration based on at least one anomalously methylated cfDNA fragment overlapping that CpG site.

For each non-cancer sample, the analytics system determines 1030, for one or more TOO labels, whether the predicted probability likelihood exceeds a TOO threshold. The TOO threshold determination is further described below in FIG. 10B.

The analytics system determines 1040 a binary threshold cutoff for predicting a presence of cancer, the binary threshold cutoff determined based on a distribution of non-cancer samples excluding one or more non-cancer samples identified as having a probability likelihood that exceeds at least one TOO threshold. Non-cancer samples, that have at least one probability likelihood for a TOO label that exceeds the TOO threshold corresponding to that TOO label, can be excluded. The analytics system can then calculate a distribution of the non-cancer samples according to a cancer score for each non-cancer sample and then from the distribution determines the binary threshold cutoff at a specificity level (e.g., 99.4-99.9% specificity). Each cancer score can be determined according to the sequencing data, e.g., the cancer score can be output by a binary cancer classifier predicting a likelihood of cancer based on methylation sequencing data, as described in FIG. 3A. In other embodiments, the cancer score can be calculated according to other methods that input at least sequencing data (e.g., methylation, single nucleotide polymorphism (SNP), DNA, RNA, etc.) and output a sample's likelihood of having cancer based on the input sequencing data.

FIG. 10B illustrates a flowchart describing a process 1005 of thresholding a TOO label for determining a binary threshold cutoff for binary cancer classification, in accordance with one or more embodiments. This process 1005 can be an embodiment of the process 1000. A binary classification for predicting between cancer and non-cancer evaluates a sample's cancer score against a determined binary threshold cutoff, wherein a sample with a cancer score below the binary threshold cutoff is determined to be non-cancer and with a cancer score at or above the binary threshold cutoff is determined to be cancer. A trained multiclass cancer classifier can evaluate a sample's methylation signal (and/or other sequencing data) to determine probabilities for a number of TOO labels classified by the multiclass cancer classifier. A TOO label can be a cancer tissue type or more particularly a cancer tissue subtype (e.g., the hematological subtypes described above). The process 1005 can be performed or accomplished by the analytics system.

The analytics system obtains 1015 a training set comprising a plurality of samples having a label of cancer or non-cancer and a holdout set comprising a plurality of samples having a label of cancer or non-cancer, i.e., either a cancer sample or a non-cancer sample, respectively. Each sample in the training set can comprise methylation sequencing data, e.g., generated according to the process 100 of FIG. 1A. In other embodiments, each training sample has other sequencing data used in tandem or in substitution of the methylation sequencing data. Moreover, each sample from the training set and the holdout set can have a cancer score. As noted above, the cancer score can be determined by the binary classifier as a likelihood that a sample has cancer given the sample's methylation sequencing data. In other embodiments, the cancer score is calculated according to other methods that input at least sequencing data (e.g., methylation, single nucleotide polymorphism (SNP), DNA, RNA, etc.) and output a sample's likelihood of having cancer according to the input sequencing data.

The analytics system, for each non-cancer training sample, determines 1025 a feature vector based on the methylation sequencing data. The analytics system can determine the feature vector for each non-cancer training sample, e.g., in a similar manner to step 320 in FIG. 3A which describes determining an anomaly score for each CpG site in a set of CpG sites considered. In some embodiments, the analytics system defines the anomaly score for the feature vector with a binary score based on whether there is an anomalous fragment in the set of anomalous fragments that encompasses the CpG site. Once all anomaly scores are determined for a sample, the analytics system can determine the feature vector as a vector of the anomaly scores associated with each CpG site considered. The analytics system can additionally normalize the anomaly scores of the feature vector based on a coverage of the sample.

The analytics system inputs 1035 the feature vector for each non-cancer training sample into a multiclass cancer classifier to generate a TOO prediction. The multiclass cancer classifier can be trained on a plurality of TOO labels, including cancer types, cancer subtypes, non-cancer, or any combination thereof. The multiclass cancer classifier can be trained according to the process 300 of FIG. 3A. The trained multiclass cancer classifier can determine, as the cancer prediction, a plurality of probabilities for the TOO labels, wherein a probability for a TOO label indicates likelihood of having a cancer corresponding to the TOO label.

In some examples, the analytics system sweeps 1045 or iterates through a range of probabilities for the TOO label as candidate TOO thresholds calculating a specificity rate and a sensitivity rate over the range of probabilities for the TOO label. The analytics system can sweep through the range of probabilities incrementally, e.g., by 0.01, 0.02, 0.03, 0.04, 0.05, etc. As the analytics system sweeps through the range of probabilities, the analytics system can filter non-cancer training samples having a probability of the TOO label at or above the candidate TOO threshold, according to the output of the multiclass cancer classifier. As a numerical example, the analytics system can consider a candidate TOO threshold of 0.35. Non-cancer training samples with a probability of the TOO label at or above 0.35 can be filtered out of the training set. The analytic system can determine an adjusted binary threshold cutoff based on the filtered training set. The analytics system can calculate a specificity rate of prediction with the adjusted binary threshold cutoff against the holdout set. The specificity can refer to an accuracy of identifying non-cancer samples as the non-cancer label. The analytics system can also calculate a sensitivity rate of prediction with the adjusted binary threshold cutoff against the holdout set. The sensitivity can refer to an accuracy of identifying cancer samples as the cancer label. In practice, the specificity rate and/or the sensitivity rate may be defined according to a true positive rate, a false positive rate, a true negative rate, a false negative rate, another statistical calculation, etc.

The analytics system determines 1055 a TOO threshold for the TOO label. The analytics system can select the TOO threshold from the candidate TOO thresholds by optimizing the calculated specificity rates and/or sensitivity rates over the range of candidate TOO thresholds. In some examples, TOO thresholds are determined or otherwise applied for certain TOO tissue type classes or subtype classes, such as hematological classes. Merely by way of example, an algorithm for computing and applying TOO-specific probability thresholds can be used to remove non-cancer samples with exceeding signals of blood disorders. The algorithm can include, for each pre-specified TOO labels, first searching through a grid of probability values, and for every value, evaluating the clinical specificity and the clinical sensitivity of a holdout set using the binary detection threshold computed after removing non-cancer samples with equal or greater probability of the specified TOO label. By iterating through the probability grids, the algorithm can identify a combination of TOO threshold values for the pre-specified TOO labels that optimizes the tradeoff between the clinical specificity and the clinical sensitivity of the holdout set. The final optimized TOO probability threshold values can be used to filter out non-cancer samples that exceeds any of the values given the TOO labels. The cleaned set of non-cancer samples can be used to compute cancer-non-cancer detection threshold. Still, in some examples, the TOO-specific thresholding can be manually set at any cutpoint, such as a specificity level (e.g., 99.4-99.9% specificity).

The analytics system tunes 1065 the binary cancer classification by pruning non-cancer training samples exceeding the TOO thresholding prior to determining the binary threshold cutoff. The analytics system can filter out non-cancer training samples from the training set according to the determined TOO threshold for the TOO label. The analytics system can set the binary threshold cutoff according to the filtered training set. For example, the analytics system determines a new binary threshold cutoff based on a filtered distribution of scores. In additional embodiments, the analytics system can determine a TOO threshold for any of the TOO labels according to steps 1010, 1020, 1030, and 1040, to tune the binary cancer classification.

### III.C.II. Stratification of Sample Distribution According to Too Signal

In one or more embodiments, the analytics system tunes the cancer classifier by stratifying the sample distribution according to TOO signal to determine a binary threshold cutoff for each stratum. The analytics system may stratify the sample distribution according to the signal for one or more TOO labels, determined according a TOO prediction output by the multiclass cancer classifier.

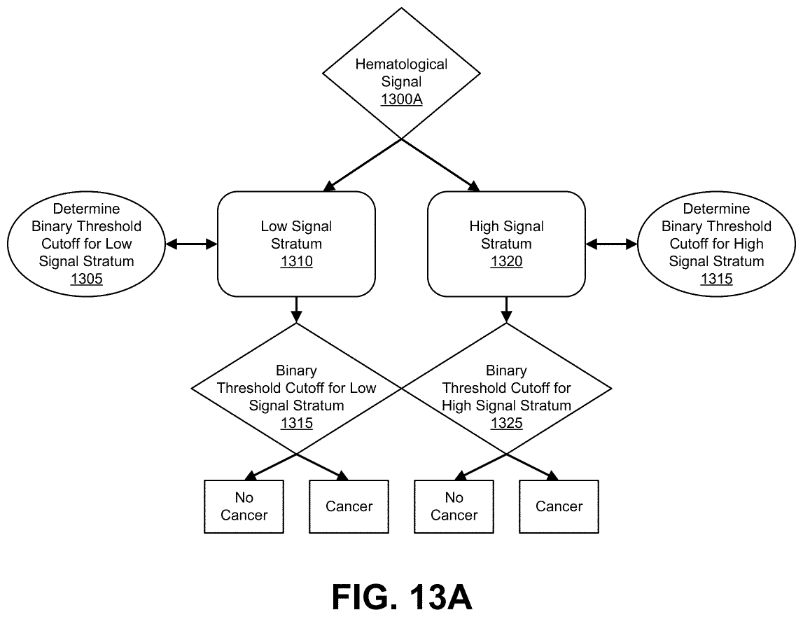

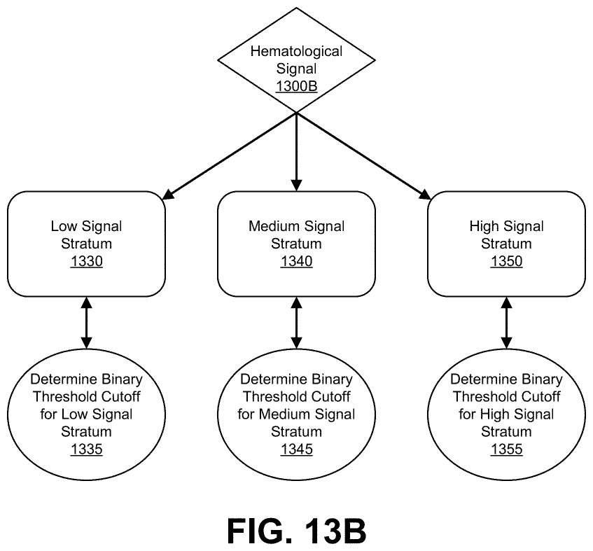

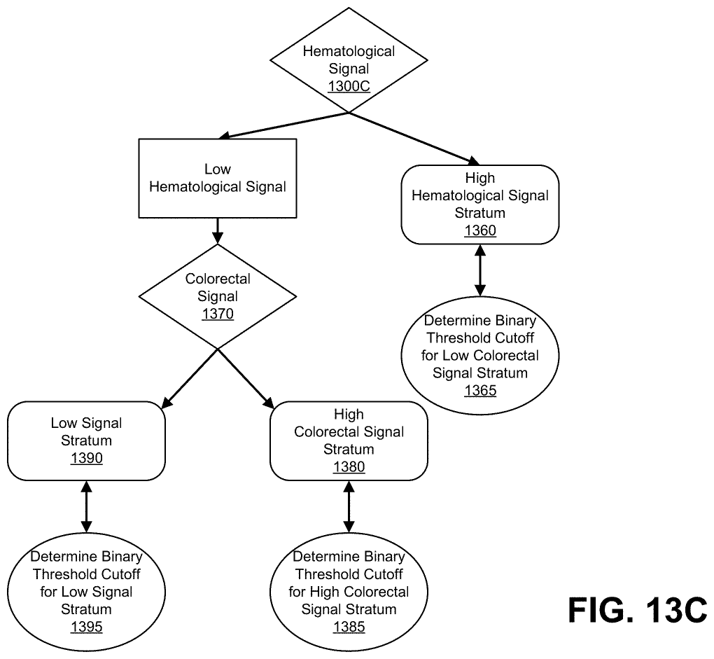

Referring now to FIG. 13A, FIG. 13A illustrates a process for stratifying hematological signals into two strata, in accordance with one or more embodiments. Although the following description describes stratification with a hematological signal, the principles may be readily applied to other TOO signals.

The analytics system stratifies 1300A a holdout set of cancer and non-cancer samples according to the hematological signal into a low signal stratum 1310 and a high signal stratum 1320. Each sample of the holdout set can have a cancer score determined by a binary cancer classifier and a TOO prediction determined by a multiclass cancer classifier. In one embodiment, hematological signal for a sample is determined according to a TOO prediction output by a multiclass cancer classifier. High tissue signal may be determined as described under Section III.C. Tuning of Cancer Classifier. In one embodiment, when considering one or more top predictions (e.g., top one, top two, etc.), high hematological signal is determined if at least one of the top predictions being considered is one of a hematological subtype (e.g., lymphoid neoplasm subtype and myeloid neoplasm subtype). Other hematological subtypes may be included. As such, if a sample has a TOO prediction with at least one of the top predictions being considered as the lymphoid neoplasm subtype or the myeloid neoplasm subtype, then the sample can be determined to have high hematological signal. Otherwise, the sample can be determined not to have high hematological signal.

The analytics system can determine a binary threshold cutoff for each stratum for predicting presence or absence of cancer of a sample. The samples in the low signal stratum 1310 are used by the analytics system to determine 1305 a binary threshold cutoff for predicting absence or presence of cancer in samples in the low signal stratum 1310. The binary threshold cutoff is determined 1305 according to a false positive budget set for the low signal stratum 1310. With cancer scores for the samples in the low signal stratum 1310, the analytics system sweeps through a range of candidate binary threshold cutoffs evaluating a true positive rate (also referred to as sensitivity) and a false positive rate at each candidate binary threshold cutoff. The candidate binary threshold cutoff with a false positive rate that is closest within the false positive budget can be determined to be the candidate binary threshold cutoff. The analytics system performs similar operations to determine 1315 a binary threshold cutoff for the high signal stratum 1320. The false positive budget for the low signal stratum 1310 and the false positive budget for the high signal stratum 1320 may be set according to a ratio of statistical true positive rates of the strata. The ratio may aim to suppress the false positive rate in the high signal stratum 1320.

For a test sample, the analytics system can place the test sample into either the low signal stratum 1310 or the high signal stratum 1320 according to hematological signal. If the test sample is placed in the low signal stratum 1310, then the analytics system applies 1315 the binary threshold cutoff for the low signal stratum 1310 to the cancer score of the test sample. If the cancer score is greater than or equal to the binary threshold cutoff for the low signal stratum 1310, then the analytics system returns a prediction of cancer presence in the test sample, and returns a prediction of no cancer otherwise. If test sample is placed in the high signal stratum 1320, then the binary threshold cutoff for the low signal stratum 1320 is applied 1325 to the cancer score of the test sample. If the cancer score is greater than or equal to the binary threshold cutoff for the high signal stratum 1320, then the analytics system returns a prediction of cancer presence in the test sample, and returns a prediction of no cancer otherwise.

FIG. 13B illustrates a process for stratifying hematological signals into three strata, in accordance with one or more embodiments. Although the following description describes stratification with a hematological signal, the principles may be readily applied to other TOO signals. The principles may also be readily extended to stratification into numbers of strata beyond three.

The analytics system can stratify a holdout set of cancer and non-cancer samples into three strata according to hematological signal: a low signal stratum 1330, a medium signal stratum 1340, and a high signal stratum 1350. Each sample of the holdout set can have a cancer score determined by a binary cancer classifier and a TOO prediction determined by a multiclass cancer classifier. In one or more embodiments, a hematological TOO label comprises multiple hematological subtypes. Any sample of the holdout set with a high tissue signal in one or more aggressive hematological subtypes can be placed into the high signal stratum 1350. Any sample of the holdout set (not already classified into the high signal stratum 1350) with a high tissue signal in one or more indolent hematological subtypes can be placed into the medium signal stratum 1340. And samples not classified in either the high signal stratum 1350 or the medium signal stratum 1340 are placed into the low signal stratum 1330. According to the principles described in FIG. 13A, the analytics system determines a binary threshold cutoff for each stratum based on a false positive budget for each stratum—a binary threshold cutoff for the low signal stratum 1330 is determined 1335, a binary threshold cutoff for the medium signal stratum 1340 is determined 1345, and a binary threshold cutoff for the high signal stratum 1350 is determined 1355. Similar to FIG. 13A, for a test sample, the analytics system identifies a stratum in which to place the test sample and applies the binary threshold cutoff for that stratum to predict the presence or absence of cancer in the test sample.

FIG. 13C illustrates a process for first stratifying hematological signals, and subsequently stratifying colorectal signals, in accordance with one or more embodiments. Although the following description describes stratification with a hematological signal and subsequently with a colorectal signal, the principles may be readily applied to other combinations of TOO signals serially evaluated.

The analytics system stratifies a holdout set of cancer and non-cancer samples according to hematological signal 1300C and subsequently a colorectal signal 1370. Each sample of the holdout set can have a cancer score determined by a binary cancer classifier and a TOO prediction determined by a multiclass cancer classifier. Similar to the principles described above in FIG. 13A, any sample of the holdout set having a high hematological signal is placed into a high hematological signal stratum 1360. Remaining samples are subsequently stratified according to colorectal signal 1370. Analogous to the hematological stratification, any sample with a high colorectal signal is placed into a high colorectal signal stratum 1380. Samples placed into neither the high hematological signal stratum 1360 nor the high colorectal signal stratum 1380 are grouped into the low signal stratum 1390. In this embodiment, the hematological signal is of a higher priority than the colorectal signal. In one or more embodiments, a plurality of TOO signals may be serially evaluated in order of priority. As such, a sample having both high hematological signal and high colorectal signal can be placed under the high hematological stratum 1360 and not under the high colorectal stratum 1380, as the hematological signal is of higher priority than the colorectal signal. According to the principles described in FIG. 13A, the analytics system determines a binary threshold cutoff for each stratum based on a false positive budget for each stratum. A binary threshold cutoff for the high hematological signal stratum 1360 is determined 1365, a binary threshold cutoff for the high colorectal signal stratum 1380 is determined 1385, and a binary threshold cutoff for the low signal stratum 1390 is determined 1395. Similar to FIG. 13A, for a test sample, the analytics system identifies a stratum in which to place the test sample and applies the binary threshold cutoff for that stratum to predict the presence or absence of cancer in the test sample.

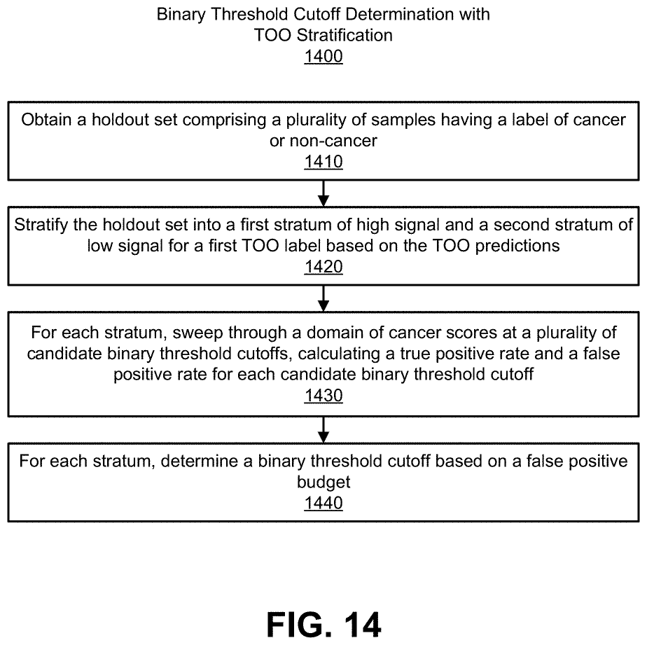

FIG. 14 illustrates a process 1400 of determining binary threshold cutoffs for TOO stratification, in accordance with one or more embodiments. Although the process 1400 is described as being performed by the analytics system, the process 1400 may more generally be performed by any computing system.

The analytics system obtains 1410 a holdout set comprising a plurality of samples classified as or having a label of cancer or non-cancer. Each sample of the holdout set can be accompanied with a cancer score, for instance, representative of a likelihood that the sample corresponds to cancer (e.g., determined by a binary cancer classifier), and a TOO prediction, for instance representative of a likelihood that the sample corresponds to cancer of a particular type of tissue (e.g., determined by a multiclass cancer classifier).

The analytics system stratifies 1420 the holdout set into a first stratum of high signal and a second stratum of low signal for a first TOO label based on the TOO predictions. In one example, the stratification uses a prediction value threshold. Any sample with a prediction value for the first TOO label in the TOO prediction at or above the prediction value threshold is classified as high signal for the first TOO label. Otherwise, the sample can be classified as low signal for the first TOO label. In another example, the analytics system considers one or more top predictions in a TOO prediction for each sample. Any sample with the first TOO label in at least one of the top predictions being considered can be classified as high signal for the first TOO label. Otherwise, the sample can be classified as low signal for the first TOO label.

In one or more embodiments, the analytics system further stratifies into a third stratum of medium signal for the first TOO label. In embodiments where signal is stratified according to prediction value, the range of prediction values may be segmented into three portions for determining high signal, medium signal, and low signal.

In one or more embodiments, the analytics system further stratifies one or more strata into additional strata according to tissue signal for one or more additional TOO labels. The additional TOO labels may be a lower priority in stratification than the first TOO label.

The analytics system, for each stratum, sweeps 1440 through a domain of cancer scores at a plurality of candidate binary threshold cutoffs, calculating a true positive rate and a false positive for each candidate binary threshold cutoff. The true positive rate can be plotted against the false positive rate to generate a receiver operator characteristic (ROC) curve.

The analytics system, for each stratum, determines 1440 a binary threshold cutoff based on a false positive budget. The false positive budget may be allocated to each stratum according to a ratio of statistical true positive rates of the strata.

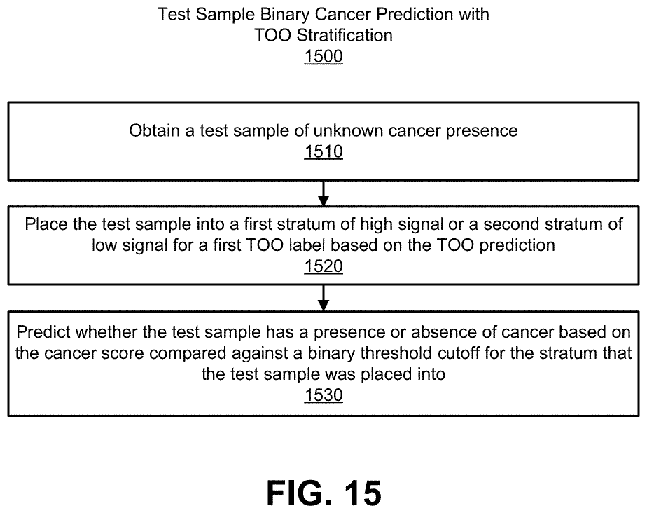

FIG. 15 illustrates a flowchart describing a process 1500 of predicting cancer presence or cancer absence for a test sample using a binary threshold cutoff determined by TOO stratification, in accordance with one or more embodiments. The process 1500 is described as being performed by the analytics system, though the process 1500 may more generally by accomplished by any computing system.

The analytics system obtains 1510 a test sample of unknown cancer presence. The test sample is accompanied with a cancer score, e.g., determined by a binary cancer classifier, and a TOO prediction, e.g., determined by a multiclass cancer classifier.

The analytics system places 1520 the test sample into a first stratum of high signal or a second stratum of low signal for a first TOO label based on the TOO prediction. Placement (or classification) is described above (for instance, with regards to stratification at step 1420 of the process 1400).

The analytics system predicts 1530 whether the test sample has a presence or absence of cancer by comparing the cancer score against a binary threshold cutoff for the stratum that the test sample was placed into. For example, if the test sample had high signal for the first TOO label and was placed into the first stratum of high signal, then the analytics system applies the binary threshold cutoff determined for the first stratum of high signal to the cancer score of the test sample. Alternatively, if the test sample was placed into the second stratum of low signal, then the binary threshold cutoff determined for the second stratum can be used. If the cancer score of the test sample is at or above the binary threshold cutoff used, then the test sample can be predicted to have a presence of cancer. Otherwise, the test sample can be predicted to be absent of cancer.

### III.D. Deployment of Cancer Classifier

During use of the cancer classifier, the analytics system can obtain a test sample from a subject of unknown cancer type. The analytics system may process the test sample comprised of DNA molecules with any combination of the processes 100, 200, and 220 to achieve a set of anomalous fragments. The analytics system can determine a test feature vector for use by the cancer classifier according to similar principles discussed in the process 300. The analytics system can calculate an anomaly score for each CpG site in a plurality of CpG sites in use by the cancer classifier. For example, the cancer classifier receives as input feature vectors inclusive of anomaly scores for 1,000 selected CpG sites. The analytics system can thus determine a test feature vector inclusive of anomaly scores for the 1,000 selected CpG sites based on the set of anomalous fragments. The analytics system can calculate the anomaly scores in a same manner as the training samples. In one embodiment, the analytics system defines the anomaly score as a binary score based on whether there is a hypermethylated or hypomethylated fragment in the set of anomalous fragments that encompasses the CpG site.

The analytics system can then input the test feature vector into the cancer classifier. The function of the cancer classifier can then generate a cancer prediction based on the classification parameters trained in the process 300 and the test feature vector. In the first manner, the cancer prediction can be binary and selected from a group consisting of “cancer” or non-cancer;” in the second manner, the cancer prediction can be selected from a group of many cancer types and “non-cancer.” In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 360 of the process 300 with another cancer classifier trained in step 370 or the process 300. The analytics system inputs the test feature vector into the cancer classifier trained as a binary classifier in step 360 of the process 300. The analytics system can receive an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier can receive the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system can determine a cancer score for a test sample based on the test sample's sequencing data (e.g., methylation sequencing data, SNP sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

### IV. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

### IV.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers can include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### IV.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

The test samples can be obtained from a cancer patient over any set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

### IV.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V. Example Results of Cancer Classifier

### V.A. Sample Collection and Processing

Study design and samples: CCGA (NCT02889978) is a prospective, multi-center, case-control, observational study with longitudinal follow-up. De-identified biospecimens were collected from approximately 15,000 participants from 142 sites. Samples were divided into training (1,785) and test (1,015) sets; samples were selected to ensure a prespecified distribution of cancer types and non-cancers across sites in each cohort, and cancer and non-cancer samples were frequency age-matched by gender.

Whole-genome bisulfite sequencing: cfDNA was isolated from plasma, and whole-genome bisulfite sequencing (WGBS; 30× depth) was employed for analysis of cfDNA. cfDNA was extracted from two tubes of plasma (up to a combined volume of 10 ml) per patient using a modified QIAamp Circulating Nucleic Acid kit (Qiagen; Germantown, Md.). Up to 75 ng of plasma cfDNA was subjected to bisulfite conversion using the EZ-96 DNA Methylation Kit (Zymo Research, D5003). Converted cfDNA was used to prepare dual indexed sequencing libraries using Accel-NGS Methyl-Seq DNA library preparation kits (Swift BioSciences; Ann Arbor, Mich.) and constructed libraries were quantified using KAPA Library Quantification Kit for Illumina Platforms (Kapa Biosystems; Wilmington, Mass.). Four libraries along with 10% PhiX v3 library (Illumina, FC-110-3001) were pooled and clustered on an Illumina NovaSeq 6000 S2 flow cell followed by 150-bp paired-end sequencing (30×).

For each sample, the WGBS fragment set was reduced to a small subset of fragments having an anomalous methylation pattern. Additionally, hyper or hypomethylated cfDNA fragments were selected. cfDNA fragments selected for having an anomalous methylation pattern and being hyper or hypermethylated, i.e., UFXM. Fragments occurring at high frequency in individuals without cancer, or that have unstable methylation, are unlikely to produce highly discriminatory features for classification of cancer status. We therefore produced a statistical model and a data structure of typical fragments using an independent reference set of 108 non-smoking participants without cancer (age: 58±14 years, 79 [73%] women) (i.e., a reference genome) from the CCGA study. These samples were used to train a Markov-chain model (order 3) estimating the likelihood of a given sequence of CpG methylation statuses within a fragment as described above in Section II.B. This model was demonstrated to be calibrated within the normal fragment range (p-value>0.001) and was used to reject fragments with a p-value from the Markov model as >=0.001 as insufficiently unusual.

As described above, further data reduction step selected fragments with at least 5 CpGs covered, and average methylation either >0.9 (hyper methylated) or <0.1 (hypomethylated). This procedure resulted in a median (range) of 2,800 (1,500-12,000) UFXM fragments for participants without cancer in training, and a median (range) of 3,000 (1,200-220,000) UFXM fragments for participants with cancer in training. As this data reduction procedure used reference set data, this stage was applied to each sample once.

### V.B. Tissue of Origin Cancer Classification

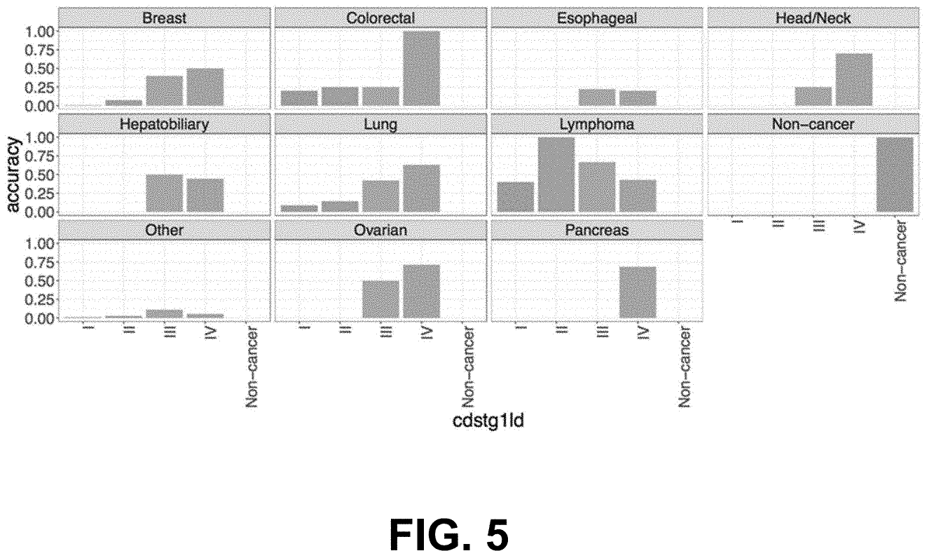

FIGS. 5-7, 11, 12A, 12B, 16A, 16B, 17, and 18 illustrate many graphs showing cancer prediction accuracy of various trained cancer classifiers, according to an embodiment. The cancer classifiers used to produce results shown in FIGS. 5-7, 11, 12A, 12B, 16A, 16B, 17, and 18 are trained according to example implementations of the process 300 described above in FIG. 3A.

The analytics system selects CpG sites to be considered in the cancer classifier. The information gain is computed for training samples with a given cancer type compared to all other samples. For example, two random variables ‘anomalous fragment’ (‘AF’) and ‘cancer type’ (‘CT’) are used. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site. For a given cancer type, the analytics system uses this information to rank CpG sites based on how cancer specific they are. This procedure is repeated for all cancer types under consideration. The ranked CpG sites for each cancer type are greedily added (e.g., to achieve approximately 3,000 CpG sites) for use in the cancer classifier.

For featurization of samples, the analytics system identifies fragments in each sample with anomalous methylation patterns and furthermore UFXM fragments. For one sample, the analytics system calculates an anomaly score for each selected CpG site for consideration (˜3,000). The analytics system defines the anomaly score with a binary scoring based on whether the sample has a UFXM fragment that encompasses the CpG site.

FIG. 5 illustrates many graphs showing cancer prediction accuracy of a multiclass cancer classifier for various cancer types, according to an example implementation. In this illustrative example, the multiclass cancer classifier is trained to distinguish feature vectors according to 11 cancer types: breast cancer type, colorectal cancer type, esophageal cancer type, head/neck cancer type, hepatobiliary cancer type, lung cancer type, lymphoma cancer type, ovarian cancer type, pancreas cancer type, non-cancer type, and other cancer type. The samples used in this example were from subjects known to have each of the cancer types. For example, a cohort of breast cancer type samples were used to validate the cancer classifier's accuracy in calling the breast cancer type. Moreover, the samples used are from subjects in varying stages of cancer.

For the breast cancer cohort, the colorectal cancer cohort, and the lung cancer cohort, the cancer classifier was gradually more accurate in accurately predicting the cancer type in subsequent stages of cancer. For the head/neck cohort, ovarian cohort, and pancreas cohort, the cancer classifier had accuracy increases in the latter stage, i.e., Stage III and/or Stage IV. For the esophageal cohort and the hepatobiliary cohort, the cancer classifier also had latter stage accuracy, i.e., Stage III and Stage IV. With the non-cancer cohort, the cancer classifier was perfectly accurate in predicting the non-cancer samples to not likely have cancer. Last but not least, the lymphoma cohort had success throughout varying stages with a peak success in accurately predicting samples in Stage II of cancer.

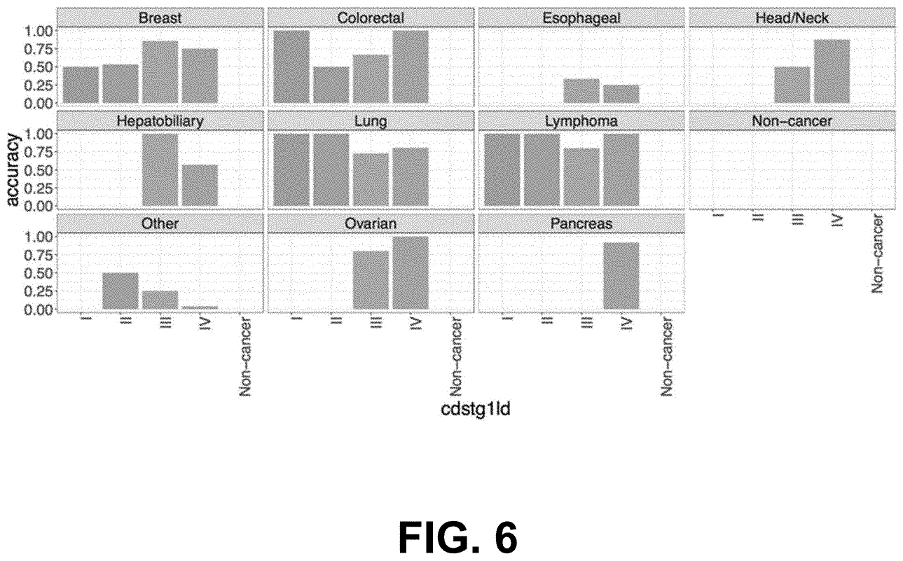

FIG. 6 illustrates many graphs showing cancer prediction accuracy of a multiclass cancer classifier for various cancer types after first using a binary cancer classifier, according to an example implementation. In this example, the analytics system first inputs the samples from many cancer type cohorts into the binary cancer classifier to determine whether or not the samples likely have or do not have cancer. Then the analytics system inputs samples that are determined to likely have cancer into the multiclass cancer classifier to predict a cancer type for those samples. The cancer types in consideration include: breast cancer type, colorectal cancer type, esophageal cancer type, head/neck cancer type, hepatobiliary cancer type, lung cancer type, lymphoma cancer type, ovarian cancer type, pancreas cancer type, and other cancer type.

In comparison to the example in FIG. 5, the analytics system showed an increase in accuracy when first using the binary cancer classifier then the multiclass cancer classifier. Among the breast cancer cohort, the colorectal cancer cohort, the lung cancer cohort, and the lymphoma cancer cohort, the analytics system had overall increases in accuracy. In particular, the analytics system had stark increases in prediction accuracy for each of those cancer types in early stages of cancer, i.e., Stage I, Stage II, and even Stage III.

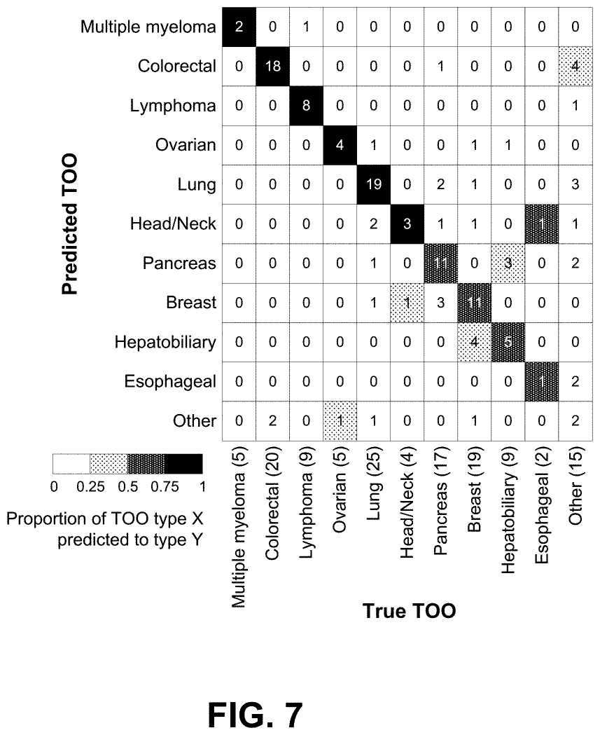

FIG. 7 illustrates a confusion matrix demonstrating performance of a trained cancer classifier, according to an example implementation. In one example of training according to the process 300, a multiclass kernel logistic regression (KLR) classifier with ridge regression penalty was trained on the derived feature vectors with a penalty on the weights, and a fixed penalty on the bias term for each cancer type. The ridge regression penalty was optimized on a portion of the training data not used in selecting high-relevance locations (using log-loss), and, once the optimum parameter was found, the logistic classifier was retrained on the whole set of local training folds. The selected high-relevance sites and classifier weights were then applied to new data. Within the CCGA training set, one fold was repeatedly held out, relevant sites on 8 of the 9 folds were selected, the hyper-parameters for the KLR classifier were optimized on the 9th set, and the KLR was retrained on 9 of 10 folds and applied to the held-out fold. This was repeated 10 times to estimate TOO within the CCGA training set. For the CCGA test set, relevant sites were selected on 9/10 folds of CCGA train, hyper-parameters were optimized on the 10th fold, and the KLR classifier was retrained on all CCGA training data and the selected sites and the KLR classifier were applied to the test set. The cancer types considered include: multiple myeloma cancer type, colorectal cancer type, lymphoma cancer type, ovarian cancer type, lung head/neck cancer type, pancreas cancer type, breast cancer type, hepatobiliary cancer type, esophageal cancer type, and other cancer type. Other cancer type included cancers with less than 5 samples collected within CCGA, such as anorectal, bladder, cancer of unknown primary TOO, cervical, gastric, leukemia, melanoma, prostate, renal thyroid, uterine, and other additional cancers.

The confusion matrix shows agreement between cancer types having samples with known cancer TOO (along x-axis) and predicted cancer TOO (along y-axis). To validate performance of the trained KLR classifier, a cohort of samples (indicated in parentheses along the y-axis for each cancer type) for each cancer type was classified with the KLR classifier. The x-axis indicates how many samples from each cohort was classified under each cancer type. For example, with the lung cancer cohort having 25 samples with known lung cancer, the KLR classifier predicted one sample to have ovarian cancer, nineteen samples to have lung cancer, two samples to have head/neck cancer, one sample to have pancreas cancer, one sample to have breast cancer, and one sample to be labeled as other cancer type. Notably, for all cancer types except other cancer type, the KLR classifier accurately predicted more than half of each cohort with particularly high accuracy for the cancer types of multiple myeloma (2/2 or 100%), colorectal (18/20 or 90%), lymphoma (8/9 or 88.8%), ovarian (4/5 or 80%), lung (19/25 or 76%), and head/neck (3/4 or 75%). These results demonstrate the predictive accuracy of the KLR classifier.

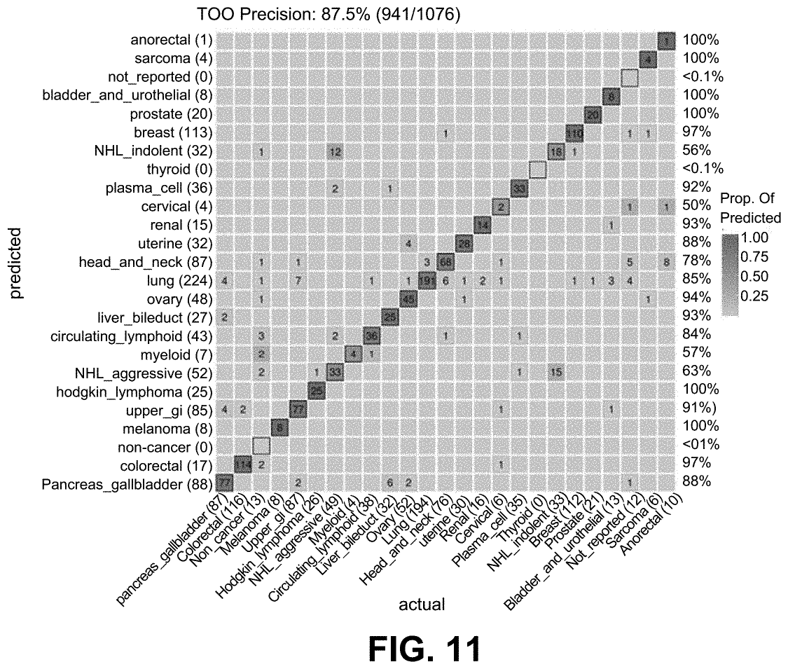

FIG. 11 illustrates a confusion matrix demonstrating performance of a trained cancer classifier with additional hematological cancer subtypes, in an example implementation. The cancer classifier may be trained according to the principles described above, for instance with regards to the cancer classifier example results of FIG. 7. The TOO labels for hematological subtypes include Hodgkin's-Lymphoma (HL), NHL aggressive, NHL indolent, myeloid, circulating lymphoma (or lymphoid), and plasma cell. Of note, the classification precision is 87.5% over 1,076.

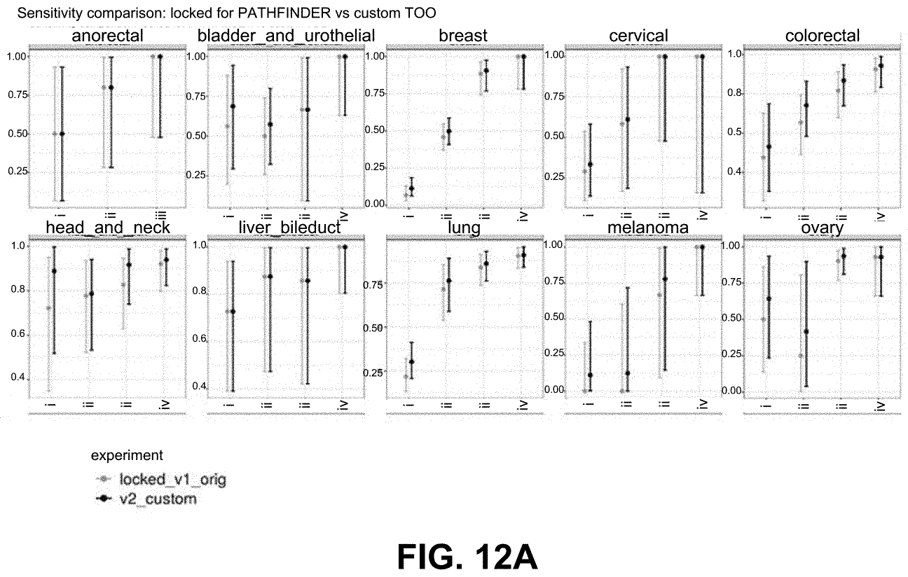

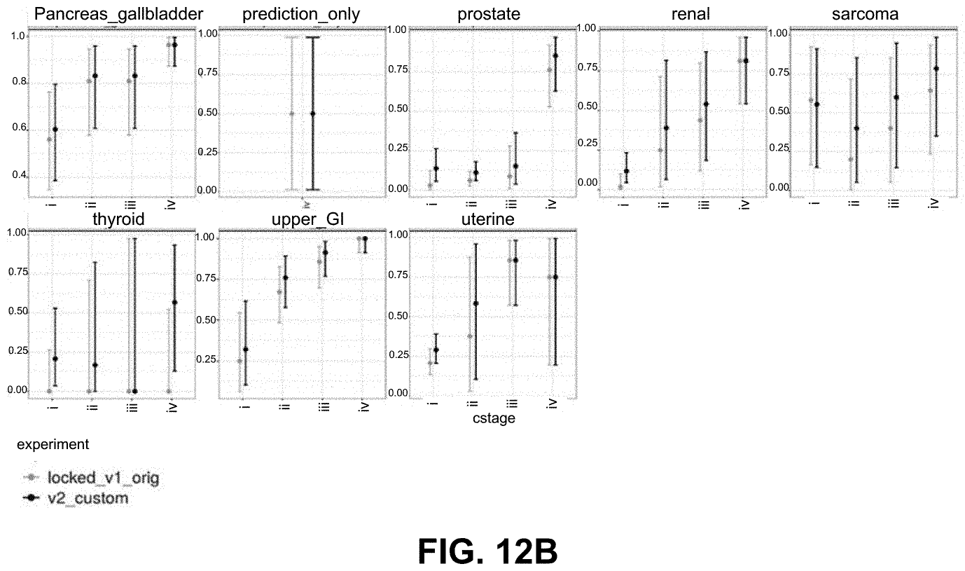

FIGS. 12A and 12B illustrate graphs showing cancer prediction accuracy for numerous cancer types over stages of cancer, in an example implementation. In this example, the cancer classifier is trained after pruning the non-cancer samples according to the process 1000 described above. The analytics system determined multiple TOO thresholds for the hematological subtypes. The analytics system excluded non-cancer samples with at least one TOO probability at or above the corresponding TOO threshold for the hematological subtypes. The graphs shown show the classification sensitivity over varying stages of cancer for cancer types: anorectal, bladder and urothelial, breast, cervical, colorectal, head and neck, liver and bile duct, lung, melanoma, ovary, pancreas and gallbladder, prostate, renal, sarcoma, thyroid, upper gastrointestinal, and uterine. A graph for each cancer type shows the prediction sensitivity over each stage of the cancer type with a first cancer classifier without TOO thresholding labeled as “locked_v1_orgi” and a second cancer classifier with TOO thresholding labeled as “v2_custom”. Notably, for many cancer types the second cancer classifier has higher prediction accuracy while maintaining a tight confidence interval, given more samples available for validation. Of particular note, there are higher prediction accuracies in many cancer types at the stage I and II levels, indicating improved prediction potential with TOO thresholding in early stage cancers.

### Example Hematologic-Specific Classifier

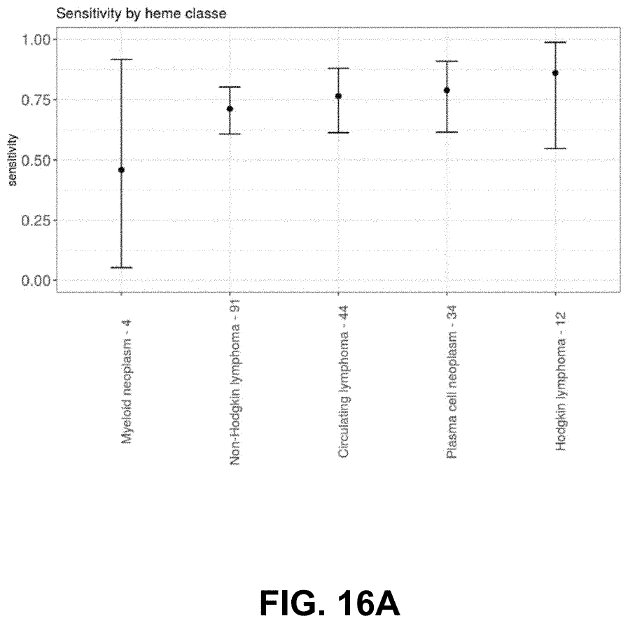

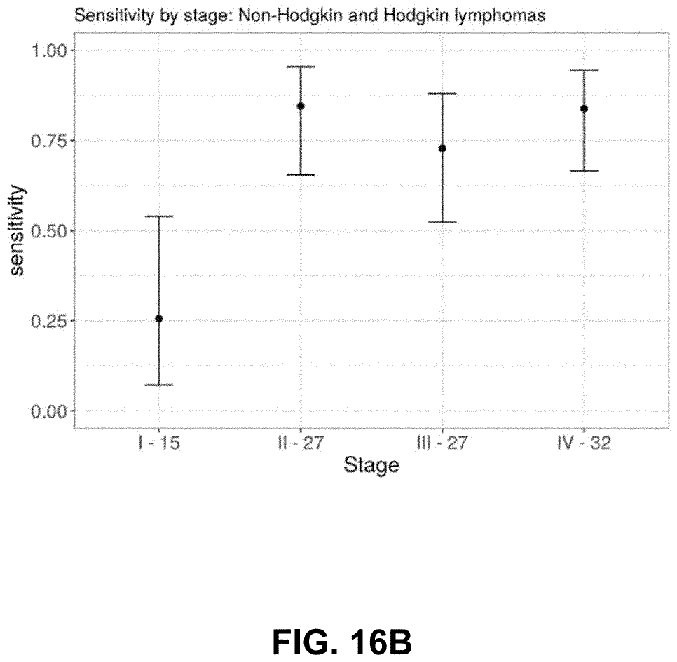

FIGS. 16A, 16B, 17, and 18 illustrate graphs showing cancer prediction accuracy for a hematological-specific cancer classifier, according to a first example implementation. First, cfDNA samples were accessed from a second pre-specified sub-study of CCGA, which was designed for targeted methylation assay validation. In this example, training set samples were used, and tumor tissue samples from an in-house tissue biopsy reference database were included for the classification model training. Specifically, the samples used to train the custom classification model for hematological malignancies were from participants enrolled with a hematological cancer diagnosis (cancer cases) and participants enrolled without a cancer diagnosis (non-cancer controls). 154 blood cell samples or tissue FFPE samples of hematological malignancies were also included. In total, cfDNA samples from 185 participants with hematological cancers and 1,998 non-cancer controls confirmed without cancer diagnosis at the one year follow-up were included for performance evaluation.

In this first example implementation, the cancer classifier is trained to distinguish between five hematological subtypes and an absence of cancer (“non-cancer”). The five hematological subtypes are myeloid neoplasm, non-Hodgkin lymphoma (NHL), circulating lymphoma, plasma cell neoplasm, and Hodgkin lymphoma (HL). A cross-validated mutual information-based algorithm was used to identify features that discriminated between the five hematological subtypes and the control class. A multinomial classifier was then trained to detect the presence or absence of cancer and predict tissue of origin among the five hematological cancers and non-cancers using 6-fold cross-validation.

The trained hematological-specific classifier achieved an overall specificity of >99.4% [95% Confidence Interval (CI): 99.0-99.7%] in controls, and a sensitivity of 74.3% [95% CI: 67.4-80.5%] for hematological cancers. FIG. 16A illustrates a graph showing the classifier's sensitivity at 99.5% specificity level across the hematological subtypes. The sensitivity of the hematological-specific cancer classifier for each hematological subtype is arranged in ascending order, with the number in the class label indicating the number of samples and the error bars showing the 95% confidence intervals. Myeloid neoplasm, having four samples classified, have a sensitivity just below 50% with a wide 95% confidence interval ranging from ˜10% to ˜90%. This lower sensitivity may be due to limited samples used in training. In the embodiment of FIG. 16A, NHL, circulating lymphoma, plasma cell neoplasm, and HL have better sensitivities than myeloid neoplasm, around 70% to 87%. Specifically, the sensitivities by hematological subtypes were 45.8% [95% CI: 5.3-91.6%] for myeloid neoplasms, 76.5% [95% CI: 61.3-88.0%] for circulating lymphomas, 86.1% [95% CI: 54.7-98.7%] for Hodgkin lymphomas, 71.3% [95% CI: 60.8-80.3%] for other Non-Hodgkin lymphomas, and 78.9% [95% CI: 61.6-91.0%] for plasma cell neoplasms.

FIG. 16B illustrates a graph showing the classifier's sensitivity at 95% specificity across stages for Hodgkin lymphomas and Non-Hodgkin lymphomas. Stage I sensitivity (out of 15 samples) is ˜25%. Stage II sensitivity (out of 27 samples) is ˜85%. Stage III sensitivity (out of 27 samples) is ˜75%. Stage IV sensitivity (out of 32 samples) is ˜85%. This graph shows a dramatic increase in sensitivity of the hematological-specific cancer classifier between Stage I and Stage II (and further). Specifically, for lymphomas (HL and NHL), the sensitivities by stages were 25.6% [95% CI: 7.2-54.0%] for stage I, 84.6% [95% CI: 65.5-95.5%] for stage II, 72.8% [95% CI: 52.4-88.0%] for stage III, and 83.9% [95% CI: 66.6-94.4%] for stage IV.

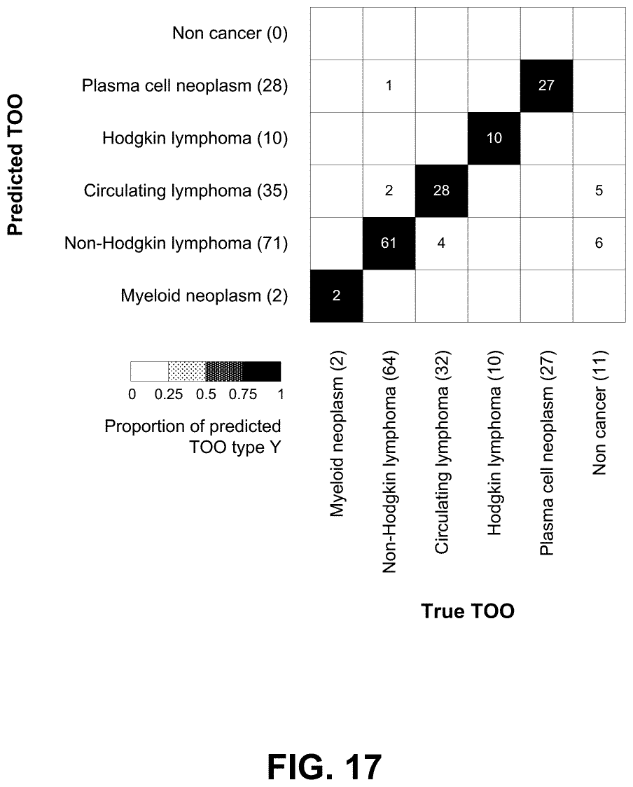

FIG. 17 illustrates a confusion matrix showing cancer prediction accuracy of the hematologic-specific cancer classifier in the first example implementation. The numbers in each box represent the total number of samples predicted. In the confusion matrix, coloring/shading corresponds to the proportion of the predicted hematological subtype, as indicated to the right of the plot. Likewise, the percentage of predictions that are correct is indicated to the right of the graph. The tissue of origin localization was assessed on cancer cases that were correctly detected by a TOO multiclass classifier as hematological cancers. As shown at FIG. 17, the hematological-specific classifier achieved an overall TOO prediction accuracy of 87.7%, with Hodgkin lymphoma and myeloid neoplasms showing the highest prediction accuracy (100%) followed by plasma cell neoplasm (96.4%), Non-Hodgkin lymphoma (85.9%), and circulating lymphomas (80%). Of the 11 non-cancer controls (0.55% of non-cancer controls) who were classified as having hematological cancers, five were predicted as circulating lymphoma, and six were predicted as other Non-Hodgkin lymphoma (<1% false positive rate), most showing confident TOO signal localizing to the predicted heme class (>50% of total probability mass).

In some examples, a low dimensional representation of the methylation features active for the final classifier can be generated using the UMAP method, which preserves the topology of high dimensional data. For example, at FIG. 9B discussed above, the UMAP embedding shows that the majority of hematological malignancies separated into five major clusters reflecting developmental lineages and disease ontogeny. The vast majority of non-cancer controls (shown using contour density at FIG. 9B) were clustered separate from the hematological cancers.

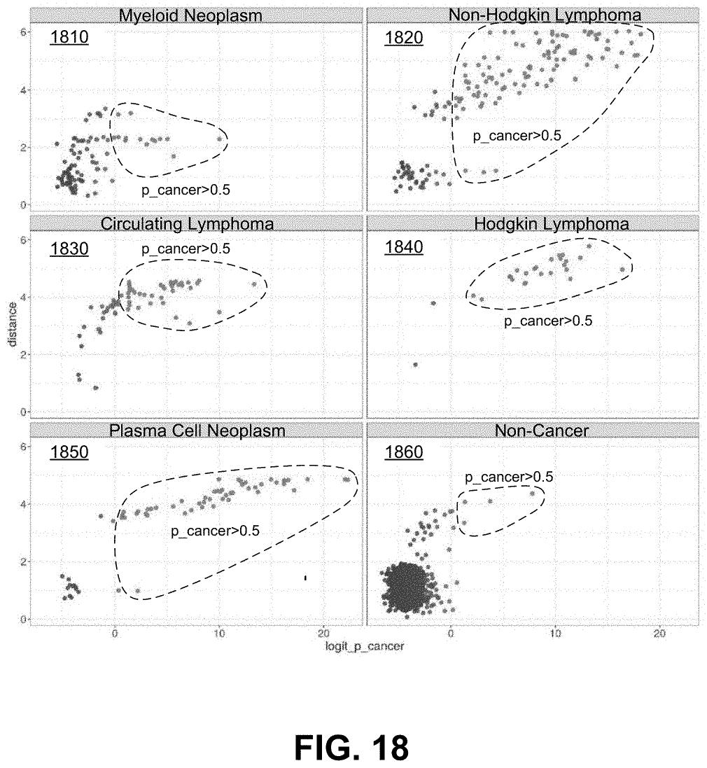

Scatter plots were then used to visualize the relationship between the spatial distribution of feature embeddings and the model's class probabilities. For example, FIG. 18 illustrates a series of graphs plotting cancer score against distance from the centroid in the UMAP embedding, in an example implementation. The UMAP embedding is the same as the UMAP embedding of FIG. 9B. The x-axis plots the logit transformed probability of a sample being cancer—i.e., logit of cancer score. The logit function (also referred to as the log-odds) is the logarithm of the odds

$$\frac{p}{1 - p} ,$$

where p is probability. The y-axis plots the Euclidean distance from the centroid of the UMAP embedding. These plots depict a correlation between the cancer score and the localization in the UMAP embedding for the various hematological subtypes. Graph 1810 depicts the correlation in the myeloid neoplasm subtype. Graph 1820 depicts the correlation in the NHL subtype. Graph 1830 depicts the correlation in the circulating lymphoma subtype. Graph 1840 depicts the correlation in the HL subtype. Graph 1850 depicts the correlation in the plasma cell neoplasm subtype. Graph 1860 depicts minimal correlation in the non-cancer samples. As shown at FIG. 18, there was a strong positive correlation between their UMAP embedding localization and classification score.

As demonstrated by the example hematological-specific classifier, the custom classifier for hematological malignancies offers a convenient way to simultaneously detect and distinguish five major hematological malignancies, which can facilitate clinical diagnosis and treatment selection. In this way, the custom classifier can achieve even more sensitive detection of multiple cancers and can be used to refine cancer detection and TOO prediction accuracy.

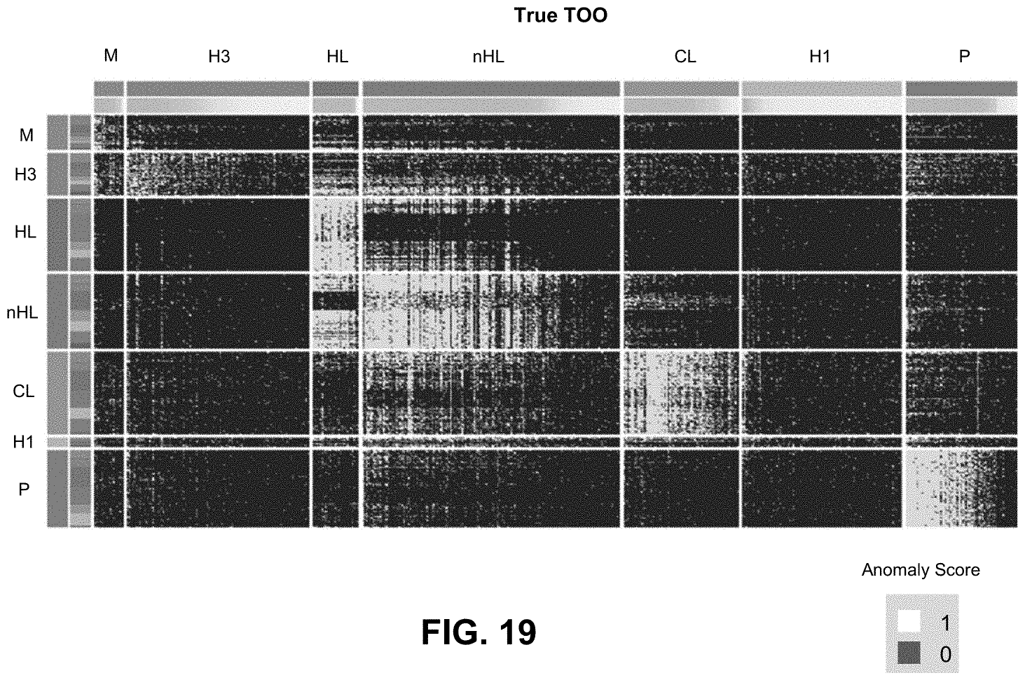

FIGS. 19, 20, and 21 illustrate graphs showing cancer prediction accuracy for a hematological-specific cancer classifier, according to a second example implementation. First, cfDNA samples were accessed from a second pre-specified sub-study of CCGA, which was designed for targeted methylation assay validation. In this example, training set samples were used, and tumor tissue samples from an in-house tissue biopsy reference database were included for the classification model training. Specifically, the samples used to train the custom classification model for hematological malignancies were from participants enrolled with a hematological cancer diagnosis (cancer cases) and participants enrolled without a cancer diagnosis (non-cancer controls). In total, cfDNA samples from 534 participants with hematological cancers were included for performance evaluation.

In this second example implementation, the cancer classifier is trained to distinguish between seven hematological subtypes and an absence of cancer (“non-cancer”). The seven hematological subtypes are myeloid neoplasm, non-Hodgkin lymphoma (NHL), circulating lymphoma, plasma cell neoplasm, Hodgkin lymphoma (HL), heme_1, and heme_3. The subtypes heme_1 and heme_3 refer to two types of hematological precursor conditions that may develop into hematological cancers such as the other hematological subtypes.

Hematological precursor conditions may include, but are not limited to, monoclonal gammopathy of uncertain significance or monoclonal B cell lymphocytosis. A cross-validated mutual information-based algorithm was used to identify features that discriminated between the seven hematological subtypes and the non-cancer class. A multinomial classifier was then trained to detect the presence or absence of cancer and predict tissue of origin among the five hematological cancers and non-cancers using 6-fold cross-validation.

FIG. 19 illustrates a graph plotting the anomaly scores of a plurality of training samples for hematological-specific cancer classification. In reference to the graph, “M” refers to the myeloid neoplasm hematological subtype, “H3” refers to the heme_3 hematological subtype, “HL” refers to the Hodgkin lymphoma hematological subtype, “nHL” refers to the non-Hodgkin lymphoma subtype, “CL” refers to the circulating lymphoma hematological subtype, “H1” refers to the heme_1 hematological subtype, and “P” refers to the plasma cell neoplasm hematological subtype. On the y-axis are several top features selected for use according to mutual information gain. As such, the first column shows each hematological subtype compared against each of the other hematological subtypes shown in the second column. Across the x-axis are training samples grouped by known hematological subtypes. For example, under column “nHL” are training samples known to be labeled the non-Hodgkin lymphoma hematological subtype. The analytics system determines an anomaly score for each of the selected features. In this implementation, the anomaly score is a binary score based on presence (shown in white) or absence (shown in grey) of an anomalously methylated fragment that covers the feature. Of note, the white regions along the main diagonal (from top left to bottom right) provides an indication to the discriminatory power in classifying the hematological subtypes. Within a row, if a feature is white spread across different samples from different hematological subtypes, there is an indication that the feature has less discriminatory power and is noisy.

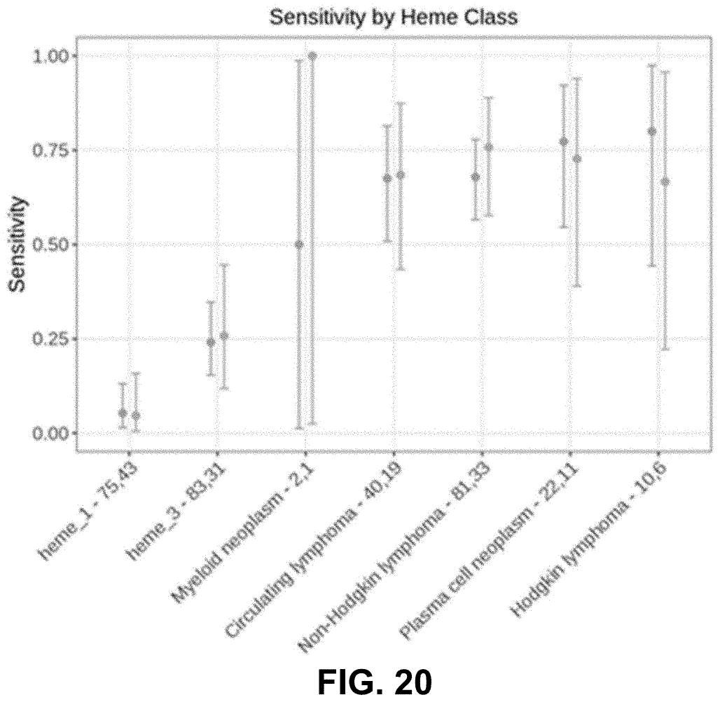

FIG. 20 illustrates a graph showing the hematological-specific cancer classifier's sensitivity at 99.5% specificity. The left set of data for each hematological subtype is for the training set used to train the hematological-specific cancer classifier; whereas, the right set of data is for the holdout set. The number of samples present in the training set and holdout set, respectively, are notated after the label for each hematological subtype across the bottom x-axis. 95% confidence intervals are shown for the sensitivities measured under the training set and the holdout set for each subtype. The heme_1 subtype had a low sensitivity for both the training set and the holdout set. The heme_3 subtype had ˜25% sensitivity for both the training set and the holdout set. The myeloid neoplasm subtype had 50% sensitivity for the training set (accurately predicted 1 in 2 training samples) and 100% sensitivity for the holdout set (accurately predicted 1 in 1 holdout sample). The circulating lymphoma subtype had ˜70% sensitivity for both sets. The non-Hodgkin lymphoma subtype had ˜70% sensitivity for the training set and ˜75% sensitivity for the holdout set. The plasma cell neoplasm subtype had ˜75% sensitivity for both sets. The Hodgkin lymphoma subtype had 80% for the training set and ˜70% for the holdout set.

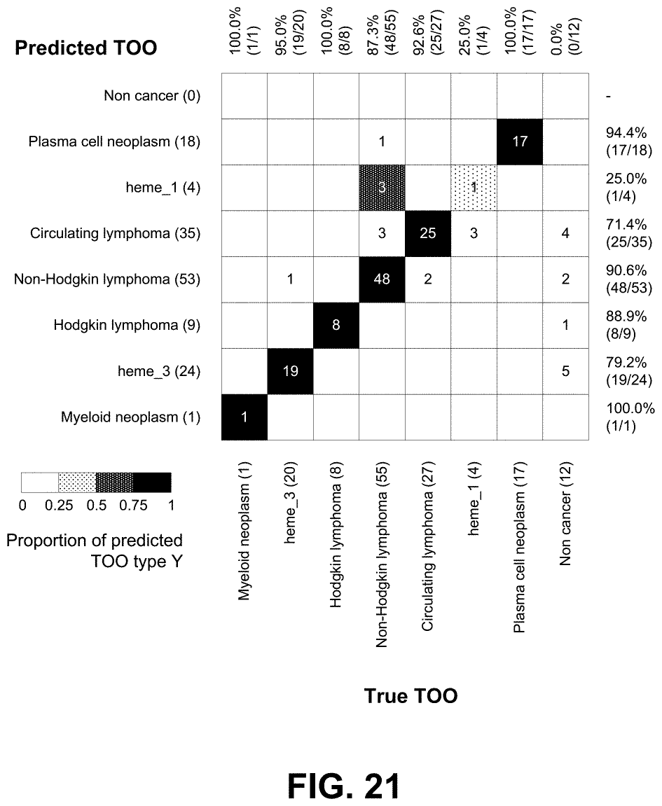

FIG. 21 illustrates a confusion matrix showing cancer prediction accuracy of the hematologic-specific cancer classifier in the second example implementation. The numbers in each box represent the total number of samples predicted. In the confusion matrix, coloring/shading corresponds to the proportion of the predicted hematological subtype, as indicated to the right of the plot. Likewise, the percentage of predictions that are correct is indicated to the right of the graph. The tissue of origin localization was assessed on cancer cases that were correctly detected by a TOO multiclass classifier as hematological cancers. As shown at FIG. 21, the hematological-specific classifier achieved an overall TOO prediction accuracy of ˜75%. Plasma cell neoplasm subtype had a prediction accuracy of 100% with 17 out of 17 known samples accurately predicted. Heme_1 subtype had a prediction accuracy of 25% with 1 out of 4 known samples accurately predicted. Circulating lymphoma subtype had a prediction accuracy of 92.6% with 25 out of 27 known samples accurately predicted. Non-Hodgkin lymphoma subtype had a prediction accuracy of 87.3% with 48 out of 55 known samples accurately predicted. Hodgkin lymphoma subtype had a prediction accuracy of 100% with 8 out of 8 known samples accurately predicted. Heme_3 subtype had a prediction accuracy of 95% with 19 out of 20 known samples accurately predicted. Myeloid neoplasm had a prediction accuracy of 100% with 1 out of 1 known sample accurately predicted.

As demonstrated by the second example hematological-specific classifier, the custom classifier for hematological malignancies and hematological precursor conditions is also capable of identifying such precursor conditions that can eventually devolve into hematological malignancies. This classification capability of precursor conditions proves helpful in identifying individuals that might later develop hematological malignancies, which can lead to even earlier clinical diagnosis and treatment selection. In this way, the custom classifier can achieve even more sensitive detection of multiple cancers and may be used to refine cancer detection and TOO prediction accuracy.

### VI. Microsimulation of Multi-Cancer Early Detection Screenings on Cancer Trajectories

### VI.A. Simulation System

In one embodiment, a simulation system performs microsimulations to model the effects of one or more early cancer detection screenings for a plurality of participants to simulate a randomized controlled trial (RCT). The microsimulation can simulate the impact of early detection screenings on individual trajectories of the participants. In particular, while most screening modalities are for single cancer types, the microsimulation performed herein can simulate the effect of a detection model on individual trajectories for participant populations having multiple types of cancer using, for example, multi-cancer early detection (MCED) screenings that are capable of detecting multiple types of cancer. In one embodiment, the microsimulations are performed by taking advantage of parallel processing techniques, and in one instance, may be performed using the programming language Julia. Julia can allow rapid prototyping and development, and also speed of execution at runtime by both compiling and providing a multi-processing environment in which the simulations can be conveniently run on multiple processes. Julia can combine the interactivity and syntax of “scripting” languages, such as R and Python, with the speed of “compiled” language such as C, which successfully solves a two-language problem. The two-language problem can include a situation where algorithms are coded in one programming language that is easy to read but rewritten in another programming language that runs fast.

In one embodiment, the MCED screening simulates the performance of one or more machine-learned cancer detection models. In one instance, the machine-learned cancer detection models can be any of the cancer detection models described in Section III of the specification. For example, the cancer detection model may be the cancer classifier trained via the process 300 in FIG. 3A or the multiclass cancer classifier trained via process 450. Moreover, the one or more cancer detection models may be configured such that each cancer detection model classifies a respective type of cancer or may be configured as a multiclass cancer detection model in which one cancer detection model classifies two or more types of cancer or may be a combination of the two.

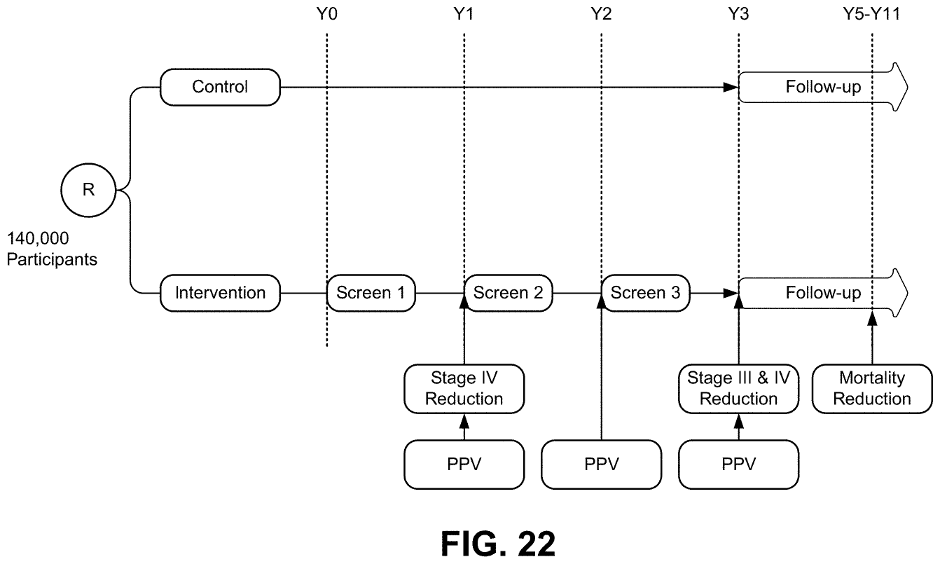

FIG. 22 illustrates the simulated RCT structure and outcome measures at different study lengths, in accordance with one embodiment. The plurality of participants can be enrolled in the RCT at an enrollment time and include a first subset of participants assigned to a control group and a second subset of participants assigned to an intervention group. For a participant in the plurality of participants, the simulation system can generate a trajectory for the participant that is a chronological timeline for cancer progression and diagnostic events of the participant. In particular, the diagnostic events may include clinical diagnosis of cancer through clinical symptoms or existing screening modalities. For a participant in the intervention group, the simulation system can further simulate MCED screenings at one or more screening times to update the trajectory of the participant. In particular, the diagnostic events of the updated trajectory may include earlier diagnosis of cancer through the MCED screenings that affect the survival time of the participant. The simulation system can compare one or more metrics related to cancer progression and diagnostics between the control group and the intervention group to determine the impact of MCED screenings. In one embodiment, the metrics include stage shift and mortality reduction.

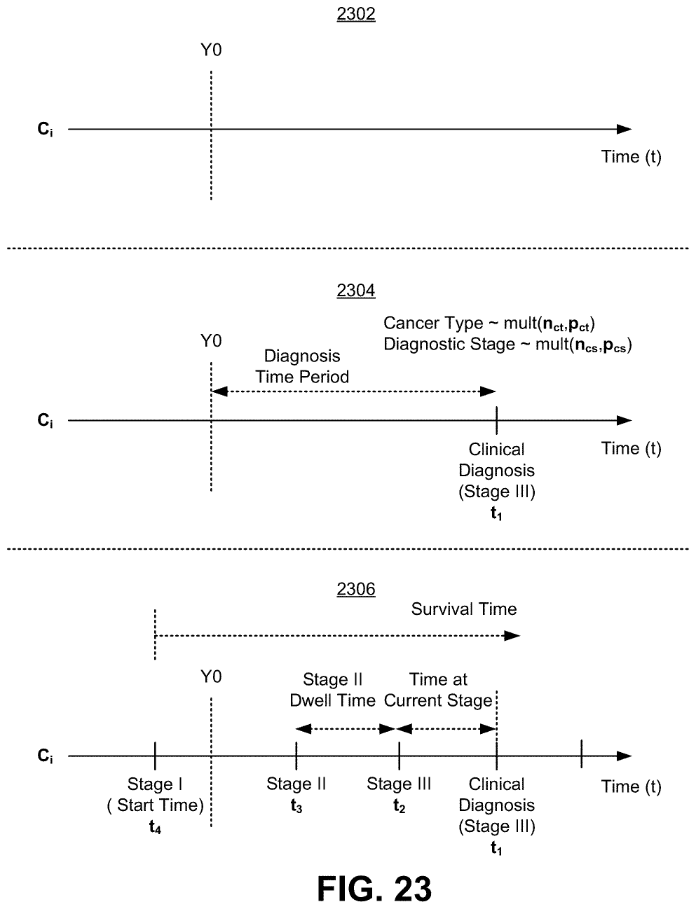

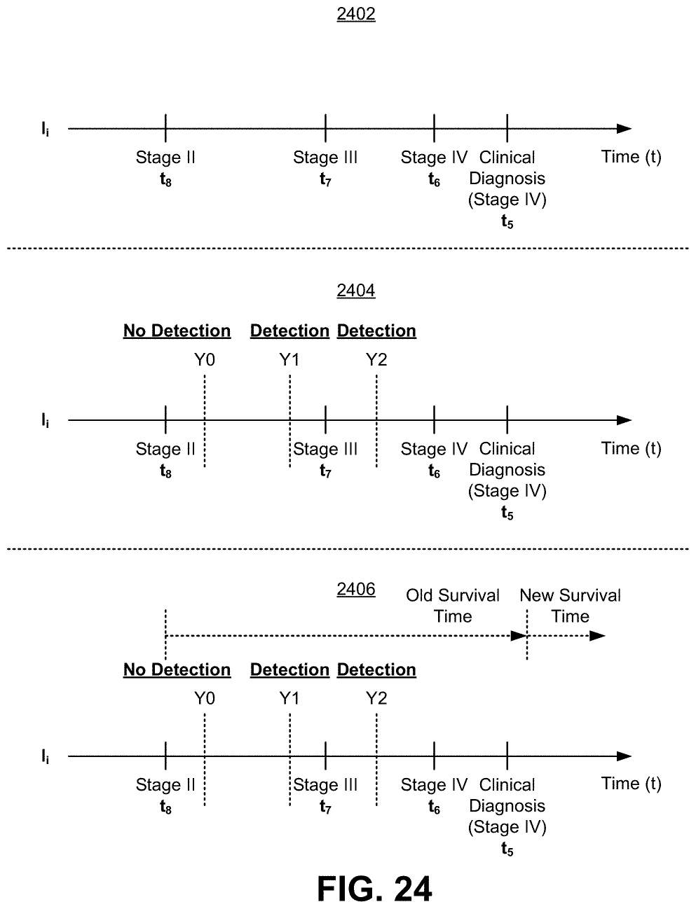

The example RCT illustrated in FIG. 22 shows a RCT for 140,000 participants enrolled in the RCT at an enrollment time Y0. A first subset of participants (70,000 participants) are assigned to a control group and a second subset of participants (70,000 participants) are assigned to an intervention group. For a participant in the plurality of participants, the simulation system generates a trajectory for the participant. For a participant in the intervention group, the simulation system simulates three MCED screenings at screening times Y0, Y1, Y2 to update the trajectory of the participant based on the performance of the MCED screenings. The details of simulating individual trajectories will be further described below with respect to FIGS. 23 and 24. In one instance, the screening times are one year apart from each other. Thus, the second screening time Y1 may be one year after the enrollment and first screening time Y0, and the third screening time Y2 may be one year after the second screening time Y1.

As described in further detail below, the simulation system determines one or more metrics related to cancer progression and diagnostics between the control group and the intervention group to determine the impact of the three MCED screenings. The metrics may be determined with respect to one or more evaluation times. In the example illustrated in FIG. 22, the simulation system may determine positive predictive values (PPV) of the cancer detection models, which indicate the proportion of participants that the model correctly estimated as having cancer out of the participants that the model estimated as having cancer. As another example, the simulation system may determine the reduction in participants diagnosed with stage IV cancer at evaluation time Y1, a one-year follow-up time after the first screening time Y0. As another example, the simulation system may determine the reduction in participants diagnosed with stage III and IV cancer at evaluation time Y3, a one-year follow-up time after the third screening time Y2. As yet another example, the simulation system may determine the mortality reduction in participants at any of the additional follow-up times Y5-Y11, where Y5 is two years after time Y3 and each sequential time is one year after the previous time.

The microsimulations described herein provide a flexible and computationally efficient framework for estimating the results of RCTs to detect clinically significant endpoints for cancer detection, stage shift, and potential mortality reduction. The microsimulations can also estimate statistical power over multiple parameter choices for the simulated RCT and account for multiple levels of variation in data. The results can be used to estimate harms as well as benefits of early cancer detection screening to patient populations with multiple types of cancer and guide formulation of appropriate standards for screenings. Moreover, instead of performing separate and multiple RCT simulations each tailored to a specific type of cancer, the microsimulation method described herein can provide a way to holistically perform a RCT simulation for multi-cancer populations using parallel processing techniques. Additionally, the microsimulation method described herein can be time efficient, cost effective, flexible and powerful, allowing parallel simulation for a plurality of subsets after simulating a plurality of cancer types instead of doing separate simulations.

### VI.B. Simulations of Individual Trajectories

The simulation system can simulate individual cancer trajectories for the plurality of participants of a RCT to determine the impact of MCED screenings. In one embodiment, the simulation system simulates multiple RCT repetitions. In one instance, the simulation system simulates 500 instances of RCT. The simulation system may execute the simulations in parallel across multiple (e.g., 96) cores. As described in detail further below, in one embodiment, the simulation system first simulates individual trajectories for the plurality of participants in the control group and the intervention group to determine cancer progression and diagnostic events under existing clinical settings without MCED screenings. The simulation system then simulates one or more MCED screenings for participants in the intervention group, and updates diagnostic events for these participants depending on the result of the screenings.

Simulations of Individual Trajectories for Clinical Diagnosis

For a participant in the plurality of participants of a RCT instance, the simulation system can generate a trajectory for the participant that is a chronological timeline for cancer progression and diagnostic events of the participant. In particular, the diagnostic events may include clinical diagnosis of cancer through clinical symptoms or existing screening modalities. Thus, this initial trajectory for participants in both the control group and the intervention group can simulate cancer progression and diagnostic events without the presence of MCED screens. In one embodiment, the trajectories for the participants are generated based on empirical data that includes cancer incidence, diagnostic stage distributions, and survival statistics from previous studies. In one instance, the empirical data is obtained from the National Cancer Institute's Surveillance, Epidemiology, and End Results (SEER) program (https://seer.cancer.gov/) for the population aged 50-79 when diagnosed with malignant primary cancer from 2006-2015 (“SEER data”), followed until December 2018 for vital status.

FIG. 23 illustrates simulation of a cancer trajectory for a participant from a control group or an intervention group, in accordance with one embodiment. In the example shown in FIG. 23, the simulation system generates a trajectory for a participant Cᵢ in the control group. However, it is appreciated that similar processes can be performed for participants in the intervention group. Moreover, the figures illustrate a timeline for simulating an individual trajectory of a participant to display cancer progression and diagnostic events on the timeline for ease of explanation. However, it is appreciated that in actual implementation, the simulation system can simulate these events using any method with or without the presence of such a timeline.

The simulation system samples a clinical diagnosis time period for the participant. In particular, the participant is expected to be clinically diagnosed with cancer after the clinical diagnosis time period for the participant has passed with respect to the enrollment time. The clinical diagnosis time period may be, for example, 0 year to 5 years, 0 year to 10 years, and the like. As defined herein, clinical diagnosis occurs when cancer is diagnosed through clinical symptoms, existing screening modalities for the types of cancer, or other existing methods for cancer diagnosis that do not incorporate early MCED screenings. In one embodiment, the clinical diagnosis time period is sampled from an exponential distribution defined by a mean rate obtained from the cancer incidence of empirical data, such as SEER data, that includes data on diagnosis time periods for previous and existing cancer patients. In such an instance, the occurrence of first cancer is assumed to be uniform over time for an individual participant. This leads to an exponential distribution to the time of cancer (e.g., first clinical cancer diagnosis) for the participant. In one instance, the exponential distribution for sampling the clinical diagnosis time period is generated by sampling a number from a uniform distribution between [0, 1] and taking the negative log of the sampled value, using the relationship that the negative log is the inverse of the cumulative distribution function for the exponential distribution. In one instance, multiple cancers are simulated for one or more subjects, since a first cancer may not be the most detectable cancer by screening.

For each participant in the plurality of participants, the simulation system can sample a cancer type and a diagnosis stage of cancer for the participant. In one embodiment, the cancer type for the participant is sampled from a multinomial distribution characterized by a set of cancer types and corresponding probabilities for sampling each cancer type. The probabilities may be determined based on the frequency of cancer types from empirical data. In one instance, the set of cancer types include 50 AJCC cancer types that are detectable via an MCED screening, including subclasses for breast cancer (HR+, HR−, HR unknown) and lung cancer (adenocarcinoma, squamous cell carcinoma, small cell, all other). In one embodiment, the diagnostic stage is also sampled from a multinomial distribution characterized by a set of cancer stages and corresponding probabilities for sampling each stage. The probabilities may be determined based on the frequency of diagnostic stages from empirical data. In one instance the set of diagnostic stages include stage I, II, III, and IV.

The clinical diagnosis time period may vary with cancer type. For example, the clinical diagnosis time period for a participant with lung cancer may be sampled from an exponential distribution with a mean 3 years, and one for a participant with breast cancer may be sampled from an exponential distribution with a mean of 1 year. Thus, the simulation system can determine a clinical diagnosis time by summing the enrollment time Y0 and the sampled clinical diagnosis time period. As defined herein, a detection time of cancer may be a time when the presence of cancer was known, and a clinical diagnosis time may be one type of a detection time in which the cancer is clinically diagnosed without the impact of early cancer detection screenings.

In the example shown in FIG. 23, the simulation system samples a clinical diagnosis time period (e.g., from an exponential distribution). The simulation system determines a clinical diagnosis time as time t₁ when the participant's cancer will be clinically diagnosed. The simulation system also samples 2304 the cancer type and diagnostic stage for the participant Cᵢ. In particular, the cancer type for participant Cᵢ is sampled from a multinomial distribution with parameters net representing the set of cancer types and p_{ct} representing the corresponding probabilities for cancer types. The diagnostic stage for participant Cᵢ is sampled from a multinomial distribution with parameters n_{cs} representing the set of cancer stages and p_{cs} representing the respective probabilities for the set of cancer stages. Based on the sampled values, participant Cᵢ is assigned to lung cancer progressed to stage III at the time of the clinical diagnosis event.

The simulation system determines the progression of one or more stages of the participant's cancer prior to the clinical diagnosis. In one embodiment, the simulation system determines one or more stages of cancer progression by determining a dwell time for each stage that indicates the time span the cancer would stay in that stage before progressing to the next stage in the absence of clinical intervention. The simulation system can sequentially use the dwell time at each stage to trace back the start time of each stage. For example, the dwell time at a current stage (stage at clinical diagnosis, e.g., stage III) can be subtracted from the clinical diagnosis time to determine the start time of the current stage. The dwell time for the previous stage (e.g., stage II) can be subtracted from the start time of the current stage to determine the start time of the previous stage, and so on, until the start time of the cancer.

In one embodiment, the dwell time per cancer type per cancer stage for a participant is sampled from a respective exponential distribution defined by a mean rate. In one instance, the simulation system accounts for various tumor growth rate scenarios and generates three sets of tumor growth rate that represents slow, fast, and aggressively fast growth rates. The exponential distribution for a dwell time for a cancer type assigned to a slow growth rate may have a mean of 3-7 years for stage I (with shorter mean times for later stages), a dwell time for a cancer type assigned to a fast growth rate may have a mean of 2-4 years for stage I (with shorter mean times for later stages), and a dwell time for a cancer type assigned to an aggressively fast rate may have a mean of 1-2 years for stage I (with shorter mean times for later stages). The time at the current stage may be uniformly distributed within the dwell time of the current stage.

In the example shown in FIG. 23, the simulation system determines 2306 the progression of one or more stages of cancer prior to the clinical diagnosis time t₁ of the participant Cᵢ. The simulation system obtains three exponential distributions that models dwell times for stages I, II, and III of lung cancer. The simulation system samples dwell times for each stage from the respective exponential distribution. In particular, the time at the current stage is further determined by uniformly sampling from the dwell time of the current stage. The simulation system subtracts the time at the current stage from the clinical diagnosis time t₁ to determine the start time of stage III as time t₂. The simulation system subtracts the dwell time for stage II from the start time of stage III to determine the start time of stage II as time t₃. The simulation system subtracts the dwell time for stage I from the start time of stage II to determine the start time of stage I and the start of the lung cancer as time t₄.

In one embodiment, the simulation system determines a clinical diagnosis time period and the cancer type and diagnostic stage at the time of clinical diagnosis to incorporate varying cancer incidence over time. Specifically, a constant cancer incidence rate over time (e.g., mean cancer incidence) for a participant may be appropriate for short lead times, but this approximation may be limited when cancer incidence changes over time (e.g., higher cancer incidence with higher age) and lead times are longer. This can be often the case for many types of cancers, as cancer incidence generally increases by age.

The simulation system can obtain sources, such as SEER data, that include intensity of cancer incidence by age. The simulation system can convert the cancer incidence into a cumulative distribution function (CDF) of cancer by age. For example, the simulation system may construct the CDF such that 25% of all cancers are diagnosed by age 80. In one instance, the simulation system generates the clinical diagnosis time period by sampling a uniform variable and applying the inverse of the constructed CDF of cancer incidence by age. Such an inverse function may be generated empirically by using function approximation techniques or any other appropriate technique.

In one embodiment, the simulation system simulates participants who are starting the screening at a predetermined age with no previous diagnosis of cancer. For example, the simulation system may simulate a uniform age of enrollment in which the plurality of participants are of the same age (e.g., 50 years old) and are simulated to be screened for a predetermined number of years (e.g., 30 years). As another example, the simulation system may simulate a plurality of participants based on a population distribution which can be drawn from population tables (e.g., individuals of age 50-80 plus individuals at younger ages based on elevated risk factors). In yet another example, the simulation system may simulate a rolling enrollment model where all comers are enrolled at a particular starting date (e.g., year 2023), but participants turning age 50 in, for example, year 2024 are allowed to newly enroll.

For a simulated participant with a given age, the simulation system can generate the clinical diagnosis time period (conditional on no cancer diagnosis before the age at the enrollment time of the study) by generating a uniform random number starting from the quantile of the age of the participant for a given age distribution. For example, for a participant of age 50, the simulation system may sample a uniform random variable between [0.6, 1], which can be done, for example, by subdividing the interval multiplicatively. The simulation system can apply the inverse of the CDF of cancer incidence by age to the sampled value. In one instance, the simulation system can simulate participants conditional on different types of clinical variables, such as gender (e.g., men, women), whether participant is a smoker, non-smoker, former smoker, and the like. The simulation system can obtain the CDF of cancer incidence for each clinical variable and generates the clinical diagnosis time for participants that are associated with the clinical variable by performing the sampling process described above and applying the inverse of the CDF of cancer incidence.

The simulation system may also assign the cancer type and diagnostic stage of a simulated participant to incorporate for variation in incidence over age. Specifically, the simulation system can extend the process of sampling the cancer type and diagnostic stage from multinomial distributions to a conditional multinomial distribution that varies over age. Thus, conditional on the clinical diagnosis time for the participant, the cancer type for the participant can be sampled from a time-varying multinomial distribution characterized by a set of cancer types and corresponding probabilities that vary by age or time. Similarly, the diagnostic stage for the participant may be sampled from a time-varying multinomial distribution characterized by a set of diagnostic stages and corresponding probabilities that vary by age.

After generating the individual trajectories, the simulation system can determine a first detection time and a first detection stage of cancer for the plurality of participants. The first detection time may be an earliest time when the cancer is correctly detected without MCED screenings, and the first detection stage may be the progression stage of cancer of the participant at the first detection time. In one embodiment, the simulation system determines the clinical diagnosis time as the first detection time of cancer for the participant, and the first detection stage as the diagnostic stage of the participant's cancer at the clinical diagnosis time. However, as further described in detail below, the microsimulations may also simulate additional non-MCED screenings, and the first detection time and the first detection stage may be determined based on the additional screenings in addition to clinical diagnosis alone.

Based on the individual trajectory, the simulation system determines a survival time for the participant based on the cancer type and first detection stage, for example, the diagnostic stage at the clinical diagnosis time. In one embodiment, the survival time is determined by linear interpolation of the empirical survival probabilities from empirical SEER data based on the cancer type and diagnostic stage for clinical diagnosis. In one embodiment, the simulation system determines the survival time by sampling a random value from a uniform distribution within a range of [0, 1] that corresponds to a survival percentile for the participant. Specifically, the empirical data can include statistical data on diagnostic survival times for previous cancer patients that indicate the time a patient survived from the time of cancer diagnosis. For example, the statistical data may show diagnostic survival times ranging from 1 year to 4 years for patients diagnosed with stage III lung cancer, with 100 percentile at 1 year, 50 percentile at 2.5 years, and 0 percentile at 4 years.

The simulation system can obtain a diagnostic survival time for the participant from cancer patients that had the same type of cancer and progression stage at clinical diagnosis (e.g., patients clinically diagnosed with stage III lung cancer), specifically the diagnostic survival time at a percentile that corresponds to the sampled survival percentile for the participant. In one embodiment, when the first detection time is the clinical diagnosis time, the survival time for the participant may be determined as the sum of the clinical diagnosis time and the diagnostic survival time determined for the participant. In the example shown in FIG. 23, the simulation system samples a survival percentile of 50% for the participant and determines the survival time of the participant Cᵢ as 2.5 years based on empirical data for stage III lung cancer diagnoses.

In one embodiment, the simulation system can incorporate varying survival times and mortality rates dependent on age as well as other clinical variables. Specifically, the simulation system can construct a multi-dimensional CDF conditioned on both age and time from clinical diagnosis for both cancer and non-cancer mortality rates. Thus, one dimension (non-cancer) of the CDF may indicate the cumulative mortality due to non-cancer causes for varying times from clinical diagnosis, and the other dimension (cancer) of the CDF may indicate cumulative mortality due to cancer causes for varying times from clinical diagnosis that are dependent on the cancer type and diagnostic stage of the participant.

For a simulated participant with a given age, the simulation system generates a date of non-cancer death based on cumulative mortality rates that vary according to age and other clinical variables. The simulation system may generate such a date by sampling a uniform random variable starting at the quantile for the participant's age at the time of enrollment and applying the inverse of the constructed CDF to the sampled value. In one instance, the simulation system may construct different CDF's conditioned on different clinical variables (e.g., smoking status may affect cardiovascular and pulmonary mortality). If the survival time computed based on the date of non-cancer death is before the age of clinical diagnosis for the participant, no alteration of the survival time for the participant is used unless MCED screening events intervene (as described below), as the participant may eventually die of non-cancer causes before the cancer is clinically diagnosed.

On the other hand, if the clinical diagnosis time of the cancer for the participant is before the age of non-cancer death, the presence of cancer may shorten the participant's lifespan and cancer mortality can be generated conditional on the participant's age at clinical diagnosis as well as the cancer type and diagnostic stage of the cancer. Moreover, participants diagnosed with cancer display an elevated non-cancer mortality post-diagnosis compared to the general population (e.g., because of treatment affecting non-cancer mortality). In such an instance, the simulation system first generates a cancer-mortality age of death and then a new non-cancer mortality age of death accounting for the fact that the participant can have survived to the clinical diagnosis age, and the simulated participant uses a more hazardous cancer-diagnosed non-cancer mortality curve. The new non-cancer mortality estimate can be substituted for the original estimate to maintain the average in the population correctly, and the survival time of the participant can be determined.

FIG. 23 illustrates an example simulation of a cancer trajectory for one participant in the control group. However, it is appreciated that this process can be repeated for other participants in the control group and intervention group, and the timeline of events may vary depending on the values that are sampled from each distribution.

Simulations of MCED Screenings for Intervention Group

For a participant in the intervention group, the simulation system can simulate early cancer detection screening and in particular, multi-cancer early detection screening, for participants in the intervention group. As described above, participants in the intervention group may already be assigned with a cancer type and diagnostic stage. The participant in the intervention group may also have simulated trajectories that indicate the participant's clinical diagnosis time and cancer progression through a process similar to that described in conjunction with FIG. 23.

FIG. 24 illustrates simulation of a cancer trajectory for a participant from an intervention group with MCED screening, in accordance with one embodiment. In the example shown in FIG. 24, the simulation system obtains 2402 a cancer trajectory generated for the participant L prior to simulation of the MCED screenings. In particular, participant L is assigned to cancer type of breast cancer and a diagnostic stage of stage IV at a clinical diagnosis time t₅. The simulation system determines the start time of stage IV as time t₆, the start time of stage III as time t₇, and the start time of stage II as time t₈.

For a participant in the intervention group, the simulation system can simulate MCED screenings at one or more screening times. The one or more screening times may occur at common times across the participants. In one embodiment, the MCED screening is simulated based on the performance of one or more machine-learned cancer detection models. For each screening event of a participant, the simulation system can determine whether the participant had cancer at the time of the screening event. If the participant had cancer at the time of the screening event, the simulation system can generate a detection estimate for the screening event of whether the cancer detection models predict the participant has cancer based on the sensitivity of the one or more cancer detection models for the cancer type and progression stage of the participant at that time. On the other hand, if the participant did not have cancer at the time of the screening event, the simulation system can generate a detection estimate for the screening event based on the specificity of the one or more cancer detection models. In one instance, the sensitivity and specificity of one or more cancer detection models are estimated from the “Circulating Cell-Free Genome Atlas (CCGA; NCT02889978) case-control study.

Specifically, the “sensitivity” of a cancer detection model can indicate the rate the cancer detection model correctly detects presence of cancer from a set of participants who have cancer and may depend on different cancer types and progression stages of the cancer at the time of the screening. Therefore, if the participant has cancer at the time of the screening event, the simulation system can generate the detection estimate based on the sensitivity of the cancer detection model being simulated for the screening. In one instance, the simulation system obtains the sensitivity of the cancer detection model (e.g., 0.7, 0.75, 0.8, 0.85) for the screening event. The test may include individual cancer detection models each directed to detecting a respective type of cancer, or a single cancer detection model that is configured to detect multiple types of cancer or a combination of both. The simulation system can sample a detection level from a range of [0, 1]. If the detection level is equal to or below the sensitivity of the cancer detection model for that cancer type and progression stage, the simulation system can generate a positive detection estimate that indicates presence of cancer. Otherwise, the simulation system can generate a negative detection estimate that indicates absence of cancer.

In one embodiment, sensitivities are assumed to vary with different cancer types and stages, and the simulation system may model the sensitivity of the one or more cancer detection models to account for stage progression of cancer in the participant's trajectory. For example, the simulation system may assume sensitivities for MCED screenings generally increase for advanced stages of cancer and assign non-decreasing sensitivities for cancer detection models as screening events are simulated for advanced stages of cancer over time. As an example, the sensitivity for a cancer detection model used to detect lung cancer in a patient may have a sensitivity of 65% if performed during stage II of cancer, 70% if performed during stage III of cancer, and 75% if performed during stage IV of cancer.

In one embodiment, to account for uncertainty remaining in the estimate of sensitivity in the study, the simulation system models a set of non-decreasing sensitivities across increasing progression stages for each cancer type that are drawn from the posterior distribution conditional on the observed data and non-informative Jeffreys prior using a Markov-Chain Monte-Carlo (MCMC) technique. The uncertainty to be propagated through the microsimulation can come from the input data being a case-control study with a finite number of cases. The observed sensitivity from this study can include detections from n attempts for each stage of the cancer. Without constraints on the true underlying sensitivity, this uncertainty may be handled with an independent binomial draw for each stage. However, in one embodiment, based on the underlying principle that cancer is a progressive disease, the unknown true sensitivities are modeled to be non-decreasing. Thus, this method can allow generation of a draw from such a distribution respecting the relative distribution of outcomes, which might not have been observed in previous microsimulation methods. A simple rejection method may be inefficient and difficult to normalize to the underlying probability distribution. However, the space of non-decreasing true sensitivities compatible with the observations can be explored efficiently by means of a Markov chain.

In one embodiment, this method constructs a starting point within the valid space (strictly increasing sensitivities, not 0 or 1, so as to not be on a boundary). This can be done in multiple ways. After, a coordinate-at-a time update can be used to propose a move for a randomly chosen stage, treating the other sensitivities as hard boundaries, as well as 0 and 1. For the hard boundary, the “mirror-move” trick may be exploited to reflect proposals outside the boundary back into the valid space. This move set satisfies the basic balance condition that a move is as likely as the reverse move. This Markov chain then samples from the appropriate probability distribution. To mix efficiently, a scale for proposals can be chosen based on the sampling uncertainty from the case-control study for each stage. By thinning the chain at an appropriate scale, approximately independent draws from the posterior distribution can be generated. Each such draw is exploited by a round of the microsimulation as the input sensitivity vector for that round. In this way, repeated rounds of the microsimulation can span the uncertainty in the input data. This methodology can generate input sensitivities that can be used and is independent from the correlation of successive screening tests. Moreover, the simulation system may also model the sensitivity such that for a given participant, a cancer type detectable at a given stage was also detectable at a more advanced stage, and a cancer type undetectable at a given stage (that is, sensitivity is zero) was undetectable at earlier stages.

On the other hand, the “specificity” of a cancer detection model can indicate the rate the cancer detection model correctly detects absence of cancer from a set of participants who do not have cancer. Therefore, if the participant does not have cancer at the time of a screening event, the simulation system can generate the detection estimate based on the specificity of the cancer detection model being simulated for the screening. In one instance, the simulation system obtains the sensitivity and/or specificity of the cancer detection model (e.g., 0.995) for the screening event. The simulation system can sample a random number from a range of [0, 1]. If the random number is equal to or below the specificity of the cancer detection model, the simulation system can generate a positive detection estimate that predicts presence of cancer. Otherwise, the simulation system can generate a negative detection estimate that predicts absence of cancer.

In the example shown in FIG. 24, a first MCED screening event occurs at time Y0 (enrollment time), a second MCED screening event occurs at time Y1, and a third MCED screening event occurs at time Y2 for the participant Iᵢ. Each screening event may be one year apart from each other. Since each screening time corresponds to a time when the participant is simulated to have cancer, the simulation system can generate a detection estimate for each screening event based on the sensitivities of the one or more cancer detection models used for the participant. The sensitivities may vary according to the stage the cancer has progressed to. In the example shown in FIG. 24, the simulation system generates a detection estimate predicting no detection of cancer for the first screening event at Y0, detection of cancer for the second screening event at Y1, and detection of cancer for the third screening event at Y2.

For each participant in the intervention group, the simulation system may sequentially generate detection estimates for one or more MCED screenings. In particular, for a screening event simulated at a time the participant had cancer, the detection estimate may be “true positive” if it correctly estimates presence of cancer or may be “false negative” if it incorrectly estimates the absence of cancer when in fact there was cancer. On the other hand, for a screening event simulated at a time the participant did not have cancer, the detection estimate may be “true negative” if it correctly estimates absence of cancer or may be “false positive” if it incorrectly estimates the presence of cancer when in fact there was no cancer present.

For each of the second subset of participants in the intervention group, the simulation system can determine whether the participant is associated with one or more true positive detection estimates for MCED screenings. The simulation system can determine a second detection time and a second detection stage for participants in the intervention group. Thus, the second detection time may be different from the first detection time assigned to the participants when generating the initial trajectory because the second detection time is when the cancer is correctly detected after one or more MCED screenings have been simulated.

In one embodiment, the simulation system determines the earliest screening event with a true positive detection estimate. If such a screening event exists and the time of the earliest screening event is earlier than the first detection time, the simulation system can determine the second detection time as the time of the earliest screening event and the second detection stage as the progression stage of cancer at the second detection time. If the participant's second detection stage is earlier than the first detection stage, a “stage-shift” can occur in which the participant's cancer is detected at an earlier stage than the stage the cancer is expected to progress at the time of clinical diagnosis alone. In the example shown in FIG. 24, the earliest true positive screening is the second MCED screening at time Y1 and the simulation system updates the second detection stage of the participant to stage II breast cancer, since Y1 is earlier than the clinical diagnosis time (first detection time) of the participant.

Specifically, once a participant in an actual RCT is assigned a positive detection estimate from a MCED screening, a follow-up investigation (e.g., clinical diagnosis) can be performed for the participant to confirm the presence or absence of cancer, so the microsimulation described herein assumes the second detection time can be relatively close to the time of the screening event. On the other hand, if there is no screening event with a true positive detection estimate or the earliest screening event with a true positive detection estimate is later than the first detection time, the second detection time for the participant may be determined to be equal to the first detection time, and thus, the second detection stage may be equal to the first detection stage.

The simulation system can update the survival time of the participants in the intervention group based on the second detection stage. For participants that have a second detection stage earlier than the first detection stage, the updated survival time may generally be greater than the survival time based on the first detection stage. Specifically, in such a case, the simulation system obtains a new diagnostic survival time based on the earlier detection stage of the participant. For example, the simulation system may obtain an empirical diagnostic survival time using an existing or newly sampled survival percentile assigned to the participant. The simulation system can determine the updated survival time as the sum of the first/second detection time and the updated diagnostic survival time. The updated survival time or cancer-specific mortality can be determined based on the condition that participants survive to the original date of diagnosis. The updated survival time or cancer-specific mortality can follow the interpolated distribution for survival based on the updated stage at detection by screening translated to the original diagnosis time. If the second detection time and the second detection stage is equal to the first detection time and the first detection stage, the survival time may not change. In the example shown in FIG. 24, the simulation system updates the survival time of the participant based on the earlier second detection stage of stage II breast cancer. The new survival time can be greater than the old survival time.

In one embodiment, in the case of “overdiagnosis” in which a cancer is detected early that would have been clinically diagnosed after another cause of death (e.g., non-cancer cause of death), a new non-cancer mortality date of death can be generated based on the earlier detection time (i.e., second detection time) using the cancer-diagnosed excess conditional mortality curve to determine whether cancer diagnosis and treatment may shorten the lifespan due to non-cancer causes. Since the participant would not have died of their cancer until the date of diagnosis, the cancer-specific mortality can on average respect this trait.

In another embodiment, the detection estimates for the MCED screenings are simulated by applying the one or more machine-learned cancer detection models to input data for the simulated participants, as described in section III of the specification. In one embodiment, the input data are one or more feature vectors of the simulated participants. For example, the simulation system may determine feature vectors for a participant in the intervention group, apply one or more trained cancer detection models to the feature vectors for the participant to generate one or more probability likelihoods the participant has cancer (single or multiple types of cancer). If the probability likelihood is above a predetermined threshold, the detection estimate may be positive, and if the probability likelihood is below a predetermined threshold the detection estimate may be negative.

In one embodiment, the simulation system may obtain feature vectors of human participants that include methylation state vectors described throughout the present specification that contain elements corresponding to sites where methylation has or has not occurred (even if those sites are not CpG sites specifically). For example, samples of human participants may be obtained and wet laboratory assays may be prepared to detect methylation and generate the methylation state vectors of the participants. The simulation system may apply a MCED model to the feature vectors including the methylation state vectors of the participants to generate detection estimates for one or more MCED screenings. In this manner, the simulation system can simulate the impact of MCED screenings on human participants given their feature vectors, without having to wait a significant amount of time to determine the various metrics for evaluation.

While FIG. 24 illustrates an example simulation of an individual trajectory for one participant in the intervention group, this process may be repeated for other participants in the intervention group, and the timeline of events may vary depending on the values that are sampled from each distribution. For example, another participant may have a clinical diagnosis time that is earlier than the times of any of the one or more MCED screening events.

Incorporation of Additional Non-MCED Screenings

In one embodiment, the simulation system simulates one or more additional non-MCED screenings that may additionally affect the first detection times and first detection stages of the participants. In one instance, the simulation system incorporates low-dose computed tomography (LDCT) screenings for lung cancer that was recommended by United States Preventive Services Task Force (USPSTF) in 2013. Specifically, the USPSTF recommends annual lung cancer screening with LDCT for adults aged 55 to 80 years who have a 30 pack-year smoking history and currently smoke or have quit within the past 15 years. Potential for LDCT screening to be added to both the control group and intervention group for simulation.

In one instance, when generating individual trajectories, the simulation system identifies a subset of participants (e.g., 10%) in each group that are eligible (e.g., participant has lung cancer) for LDCT screening and identifies a further subset of participants (e.g., 5%) within that subset that adopts LDCT screening. The LDCT screening can be assumed to occur every year from enrollment time. In one embodiment, similar to the method for simulating MCED screenings described in conjunction with FIG. 24, the simulation system assigns a detection estimate at each LDCT screening time for participants that adopted LDCT in the control group and the intervention group based on the sensitivities and specificities of LDCT screenings found through empirical studies. In one instance, the sensitivities of the LDCT screening ranged from 84.9% to 78.6% and the specificities of the LDCT screening ranged from 87.2% to 94.7%. For each participant in the control group and the intervention group selected to adopt LDCT screenings, the simulation system may sequentially generate detection estimates for a set of annual LDCT screenings for the identified participants in both the control group and the intervention group.

The simulation system may further determine the first detection time and first detection stage based on the LDCT screenings in addition to clinical diagnosis. Specifically, the simulation system can determine whether the participant is associated with one or more true positive detection estimates for LDCT screenings. If a participant is associated with a true positive LDCT detection estimate, the simulation system may determine whether the stage of the cancer at the time of such earliest LDCT screening is earlier than the diagnostic stage of cancer at the clinical diagnosis time. If the progression stage of cancer at the time of LDCT screening is earlier, the simulation system may determine the first detection time as the time of the LDCT screening and the first detection stage as the stage of cancer at the time of LDCT screening, and determine the survival time based on the detection stage of the LDCT screening. Otherwise, the simulation system may keep the first detection time as the clinical diagnosis time and the first detection stage as the diagnostic stage of the cancer.

Determination of Performance Metrics

As described below in further detail with respect to the experimental results section, the simulation system can determine one or more metrics related to cancer progression and diagnostic events after one or more RCT simulations are performed. In one embodiment, the one or more metrics for each group are determined based on the first detection time and first detection stage of participants in the control group and the second detection time and second detection stage of participants in the intervention group. Thus, cancer in participants in the control group can be assumed to be detected at the first detection time at a stage progressed to the first detection stage. On the other hand, cancer in participants in the intervention group can be assumed to be detected at the second detection time at a stage progressed to the second detection stage.

### VI. C. Experimental Results

The experimental results described in this section are a result of 500 RCT simulations. Each RCT simulation assumed a population of 140,000 participants total, 70,000 participants assigned to the control group and 70,000 participants assigned to the intervention group with a 6% annual dropout rate. Sampling of cancer incidence, dwell times, clinical diagnosis times were based on SEER empirical data. Three MCED screenings were simulated from the enrollment time Y0, with each subsequent screening happening in one-year intervals. The sensitivity of the MCED screening was determined based per cancer type per stage based on SEER empirical data, and the sensitivity of the MCED screening was set as 99.5%.

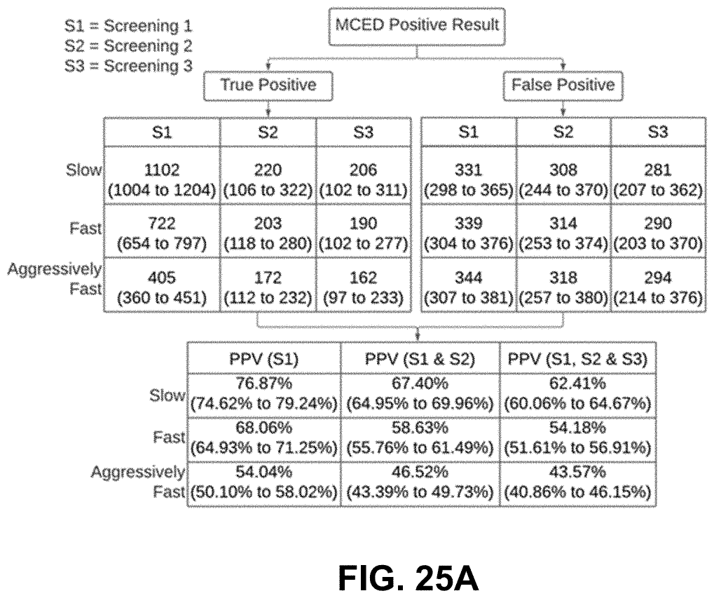

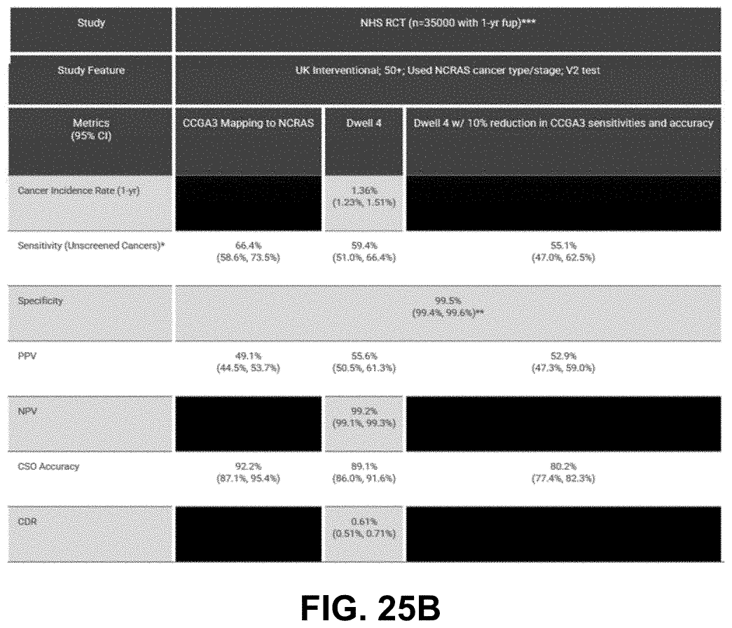

FIG. 25A are experimental results illustrating true positive, false positive, and positive predicted value (PPV) of an example RCT simulation. In one embodiment, the one or more metrics include statistics related to true positive detection estimates, false positive detection estimates, and PPV's of the RCT simulation. The PPV indicates the proportion of positive detection results that were true positive estimates. The label “S1” indicates the first MCED screening, “S2” indicates the second MCED screening, and “S3” indicates the third MCED screening, while “Slow,” “Fast,” and “Aggressively Fast” indicate cancer types assigned to each respective tumor growth rate.

As shown in FIG. 25A, the number of false positive estimates ranged from 331 to 344 in S1, 308 to 318 in S2, and 281 to 294 in S3 under different tumor growth rates. This reflected the overall false positive rate of 0.5% after accounting for dropouts and deaths during the trial process. The cumulative PPV's were 0.0.7687 (95% confidence interval (CI): 0.7462 to 0.7924), 0.6740 (95% CI: 0.6495 to 0.6996), and 0.6241 (95% CI: 0.6006 to 0.6467) after 51, S2, and S3, respectively, under a slow tumor growth rate. Given that fewer cancers were intercepted with a faster growth rate, PPV's declined with a faster tumor growth rate. For example, the PPV's were 0.6806 (95% CI: 0.6493 to 0.7125), 0.5863 (95% CI: 0.5576 to 0.6149), and 0.5418 (95% CI: 0.5161 to 0.5691) with the fast growth rate, and were 0.5404 (95% CI: 0.5010 to 0.5802), 0.4652 (95% CI: 0.4339 to 0.4973) and 0.4357 (95% CI: 0.4086 to 0.4615) with the aggressively fast growth rate.

FIG. 25B are experimental results illustrating negative predicted value (NPV), sensitivity, specificity, cancer detection rate (CDR), CSO accuracy, and cancer incidence rate for simulated example MCED screening based on CCGA3 clinical study. The NPV indicates the proportion of negative detection results that were true negative estimates. The CDR indicates the ratio of true positive detection estimates to the total number of samples (i.e., sum of true positive, false positive, true negative, false negative samples). The CSO accuracy indicates the proportion of correct cancer signal predictions among true positive samples. The cancer incidence rate indicates the proportion of number of cancers occurred out of the total number of participants.

As shown in FIG. 25B, the first column indicates the sensitivity, PPV, and CSO accuracy of a MCED model based on a CCGA3 clinical study for NCRAS cancers. The sensitivity of the model was 0.664 (95% CI: 0.586 to 0.735), the specificity of the model was 0.995 (95% CI: 0.994 to 0.996), the PPV was 0.491 (95% CI: 0.445 to 0.537), and the CSO accuracy was 0.922 (95% CI: 0.8781 to 0.954). The second column indicates the metrics for cancers of aggressively fast tumor growth rates. The cancer incidence rate was 0.0136 (95% CI: 0.0123 to 0.0151), the sensitivity was 0.594 (95% CI: 0.510 to 0.664), the PPV was 0.556 (95% CI: 0.505 to 0.613), the NPV was 0.992 (95% CI: 0.991 to 0.993), the CSO accuracy was 0.891 (95% CI: 0.86 to 0.916), and the CDR was 0.0061 (95% CI: 0.0051 to 0.0071). The third column indicates the metrics for aggressively fast tumor growth rates with a 10% reduction in CCGA3 sensitivities and accuracy. The sensitivity was 0.551 (95% CI: 0.470 to 0.625), the PPV was 0.529 (95% CI: 0.473 to 0.590), and the CSO accuracy was 0.802 (95% CI: 0.774 to 0.823).

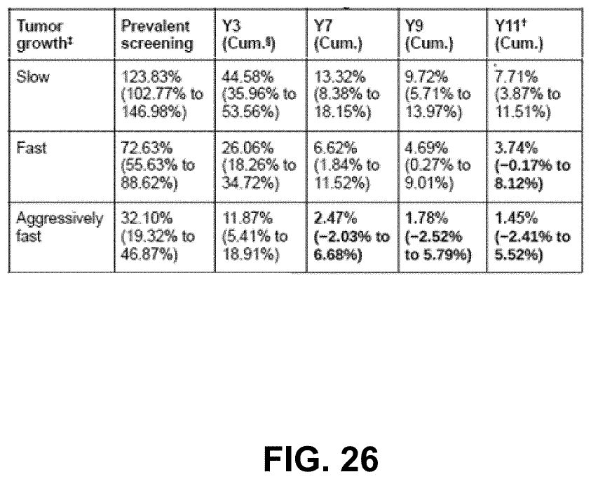

FIG. 26 are experimental results illustrating proportion of cancers detected in the intervention group compared to the control group after three MCED screenings. In one embodiment, the one or more metrics also include statistics related to the number of cancers detected in the plurality or participants. In FIG. 26, Y3 is three years after, Y7 is seven years after, Y9 is nine years after, and Y11 is eleven years after the enrollment time Y0. Each column in FIG. 26 represents the proportion of more cancers detected in the intervention group compared with the control group for different tumor growth rates. After three rounds of screening, there were 11.87% (95% CI: 5.41% to 18.91%) to 44.58% (95% CI: 35.96% to 53.56%) more cancers detected in the intervention group under different tumor growth rates. The excessive cancers were mainly due to the prevalent screening where undetectable cancers by clinical diagnosis in the first year were detected earlier by the MCED screenings. With extended follow-up times, there were “catch-up” cancers in the control group which led to smaller differences between the two arms. For example, at time Y11, there were 1.45% (95% CI: −2.41% to 5.52%) to 7.71% (3.87% to 11.51%) more cancers cumulatively detected in the intervention group.

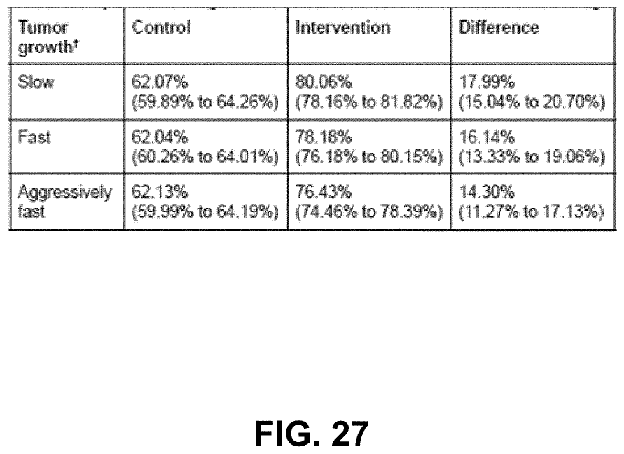

FIG. 27 are experimental results illustrating proportions of stage I and II cancers after three rounds of MCED screening. In one embodiment, the one or more metrics also include statistics related to detection of cancer at earlier progression stages. In particular, FIG. 27 illustrates the proportion of stage I and II cancers detected in the control group and the intervention group by time Y3. In the control group, the proportion of stage I and II cancers was approximately 62%. The LDCT screening of lung cancer was implemented for both groups and relatively increased the early-stage lung cancers detected in the control group. In the intervention group, tumor growth rate affected results. For instance, under slow tumor growth rate, 80.06% (95% CI: 78.16% to 81.82%) of the detected cancers were stage I and II, which resulted in an increase of 17.99% (95% CI: 15.04% to 20.70%) compared with the control group. For the fast and aggressively fast tumor growth rates, the proportions of stage I and II cancers were 78.18% (95% CI: 76.18% to 80.15%) and 76.43% (95% CI: 74.46% to 78.39%), respectively. The differences were then 16.14% (95% CI: 13.33%, 19.06%) and 14.30% (95% CI: 11.27% to 17.13%), respectively between the control group and the intervention group.

The results shown in FIG. 27 illustrate the effect of stage shift by performing MCED screenings. Specifically, the MCED screenings shifted the proportion of detected stage I and II cancers to be more than 76% under all tumor growth rates and resulted in a significant increase in early-stage cancers compared to the control group. Moreover, the results illustrate that implementing MCED screenings can help detect early-stage cancers that might well be asymptomatic and might go clinically undiagnosed until after the cancer has progressed to advanced stages.

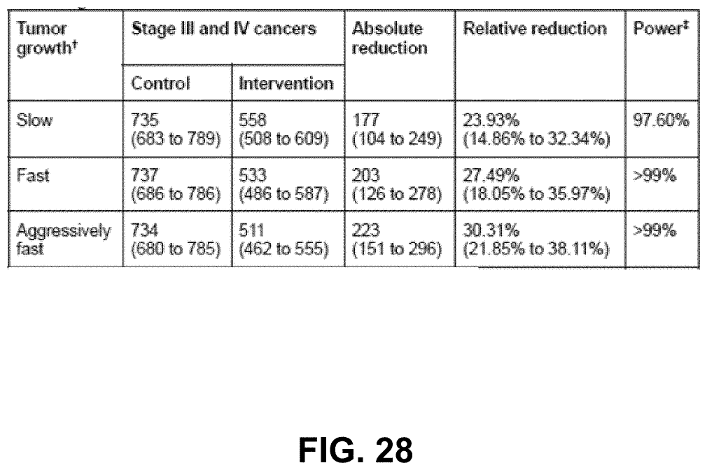

FIG. 28 are experimental results illustrating absolute and relative reductions of stage III and IV cancers after the three MCED screenings. In one embodiment, the one or more metrics include statistics related to detection of cancer at advanced progression stages. In particular, FIG. 28 illustrates the absolute and relative reductions of stage III and IV cancers between the control group and the intervention group after the three MCED screenings. Under slow tumor growth rates, 735 (95% CI: 683 to 789) stage III and IV cancers were detected in the control group, compared to 558 (95% CI: 508 to 609) in the intervention group. The absolute reduction was 177 (95% CI: 104 to 249) and the relative reduction was 23.93% (95% CI: 14.86% to 32.34%). The power to detect a significant reduction in stage III and IV cancers was 97.60%. Under fast tumor growth rates, the absolute reduction was 203 (95% CI: 126 to 278) and the relative reduction was 27.49% (95% CI: 18.05% to 35.97%). Under aggressively fast tumor growth rates, the absolute and relative reductions were 223 (95% CI: 151 to 296) and 30.31% (95% CI: 21.85% to 38.11%). The powers were greater than 99% for both fast and aggressively fast tumor growth rates.

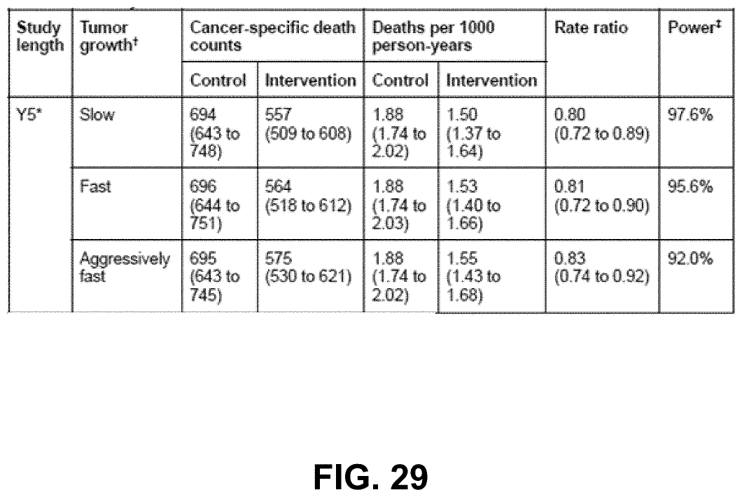

FIG. 29 are experimental results illustrating mortality reduction under different types of tumor growth rates. In one embodiment, the one or more metrics include statistics related to cancer deaths and mortality reduction at one or more evaluation times. Specifically, a cancer death at an evaluation time may occur if the survival time of a participant is less than the evaluation time, meaning that the participant did not survive until the evaluation time. In the results of FIG. 29, the evaluation time is Y5, five years after the enrollment time Y0. The cancer-specific death counts denote the number of deaths by cancer at this evaluation time. The deaths per 1000 person-years denotes the number of deaths per 1000 person-years, where person-years are calculated from the enrollment time Y0 to the date of cancer death or censoring due to dropout or end of study, whichever came first. The rate ratio was defined as the ratio of cancer-specific death rates between the control group and the intervention group.

The results in FIG. 29 illustrates cancer-specific mortality at evaluation time Y5. Under slow tumor growth rates, the death counts were 694 (95% CI: 643 to 748) and 557 (95% CI: 509 to 608) in the control group and the intervention group, respectively. This equates to a death rate of 1.88 (95% CI: 1.74 to 2.02) and 1.50 (95% CI: 1.37 to 1.64) deaths per 1000 person-years and a rate ratio of 0.80 (95% CI: 0.72 to 0.89). The power of a significant reduction in cancer-specific mortality with a two-sided exact test was 97.6%. A faster tumor growth rate led to a slightly smaller effect size and a lower power. Rate ratios were 0.81 (95% CI: 0.72 to 0.90) and 0.83 (95% CI: 0.74 to 0.92) and powers dropped to 95.6% and 92.0% under the fast and aggressively fast tumor growth rates. Thus, FIG. 29 illustrates the effect of MCED screenings on potential mortality reduction at one or more evaluation times.

As described in conjunction with various experimental results presented in this section, the method of simulating RCT's herein can provide a computationally efficient way for evaluating MCED screenings to detect clinically significant endpoints for cancer detection, stage shift, and mortality reduction. Moreover, most existing screening modalities are for single cancer types, and while simulations may be performed to simulate the impact of these existing screening modalities, it is difficult to estimate the impact of early detection screenings on a population of participants with multiple-cancer types using machine-learned detection models configured to predict multiple types of cancer. Instead of having to perform multiple and separate RCT simulations for each type of cancer, the microsimulation method described herein can provide a way to holistically perform a RCT simulation for multi-cancer populations. This can also be facilitated by using multiple or parallel cores and parallel processing through a highly efficient and powerful programming language such as Julia.

### VI.D. Process of Computer-Simulation of RCT's

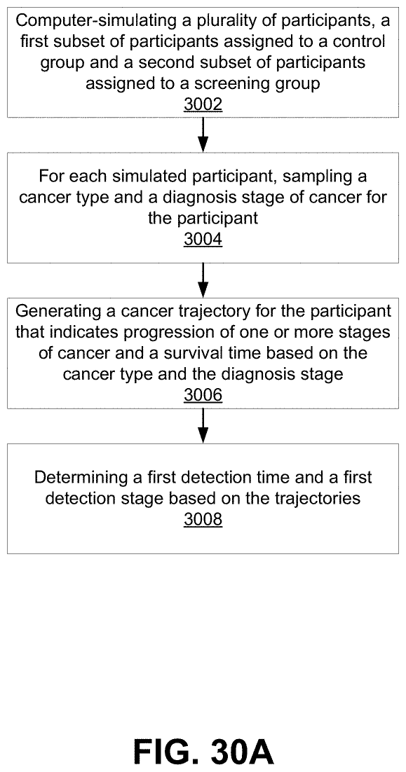

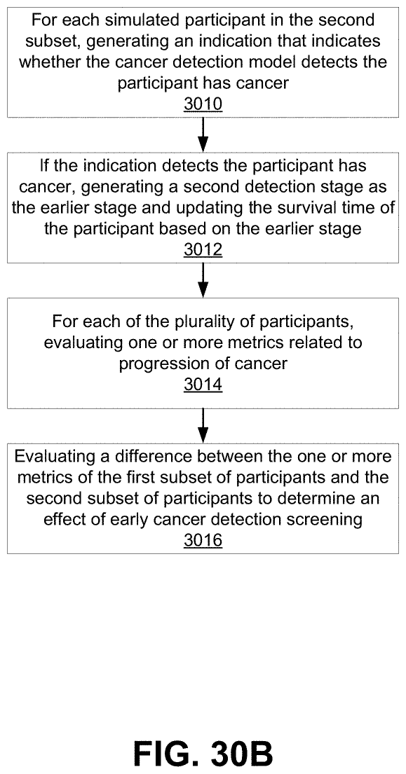

FIGS. 30A and 30B illustrate a flowchart describing a process of computer-simulating a RCT to determine impacts of MCED screenings, in accordance with an embodiment. One or more of the steps in FIGS. 30A-30B may be performed by the simulation system but embodiments are not limited hereto.

The simulation system computer-simulates 3002 a plurality of participants. A first subset of the participants can be assigned to a control group and a second subset of participants can be assigned to an intervention group. For a simulated participant in the plurality of participants, the simulation system samples 3004 a cancer type and a diagnostic stage of cancer for the participant. The participant can be clinically diagnosed with the cancer type progressed at the diagnostic stage after a diagnosis time period for the participant has passed with respect to an enrollment time. The simulation system further generates 3006 a cancer trajectory for the participant that indicates progression of one or more stages of cancer and a survival time based on the cancer type and the diagnostic stage for the participant. The simulation system determines 3008 a first detection time that indicates a time when the cancer of the participant was correctly detected at a first detection stage equal to or earlier than the diagnostic stage.

For a simulated participant in the second subset of participants of the intervention group, the simulation system can simulate early cancer detection screening by one or more machine-learned cancer detection models for at least one screening time. Specifically, the simulation system generates 3010 an indication for the participant that indicates whether the machine-learned cancer detection models detect the participant has cancer. If the indication of the cancer detection models detects the participant has cancer and the participant has the earlier stage of cancer at the at least one screening time, the simulation system generates 3012 a second detection stage as the earlier stage and updates the survival time of the participant based on the earlier stage.

For the plurality of participants, the simulation system evaluates 3014 one or more performance metrics related to progression of cancer. The simulation system evaluates 3016 a difference between the one or more metrics of the first subset of participants and the second subset of participants to determine an effect of early cancer detection screening on a multi-cancer population.

### VII. Evaluating Effects of Early MCED Screening by Integrating Over Time Through Integral Model

### VII.A. Integration System

The microsimulations described in section VI can simulate participants and model each participant's individual trajectory to determine the performance of MCED screenings via various evaluation metrics, such as the number of cancers detected, reduction in mortality due to stage-shift, and expected improvement in life expectancy. Thus, microsimulations can provide insight into how many cancers are detected due to such early screenings and the benefits of doing so for a given population with respect to lead time and mortality benefit. Moreover, as also described throughout section VI, microsimulations can additionally incorporate varying cancer incidence with age or time to determine how MCED screenings affect individuals when incidence of cancer is known to change with age or time. Often times, entities may request information on how these metrics change as a function of time or age of the participants, especially since cancer incidence changes with age.

In one embodiment, an integration system can integrate, over future time, to determine various metrics for MCED screenings as a function of age or time (or a combination of both). For example, the integration system may determine how many future incidences of cancers would be intercepted at each MCED screening as a function of time without individually simulating trajectories of participants. In other words, when it is feasible in some cases to integrate out future cancer incidence and mortality rates over the population of individuals, the integration system may integrate over diagnosis times to determine the number of “intercepted” detections and integrate over future mortality and lead time to obtain expected improvement in life expectancy. By doing so, the integration system can provide insight into how varying cancer incidence over time would affect MCED screening detection while saving computational resources that might otherwise be required to perform a number of microsimulations.

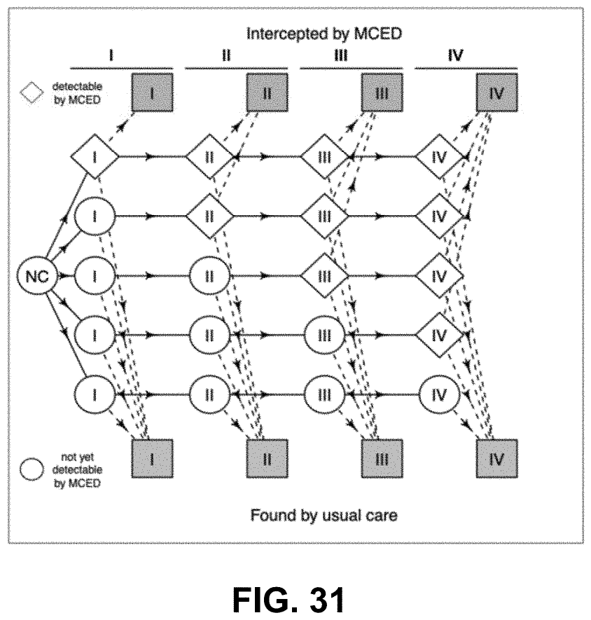

FIG. 31 illustrates an interception model approach that identifies different subpopulations of detectability, according to one embodiment. In one embodiment, the integration system obtains distinct subpopulations of detectability based on which stage cancer is first detectable by MCED screenings and how effective the MCED screenings are in intercepting cases before clinical diagnosis time. As defined herein, an “interception” or an “intercepted case” is an event in which a MCED screen detected the presence of cancer for a participant before an estimated or actual clinical diagnosis time of the participant. For example, due to the presence of one or more MCED screenings, a participant with lung cancer may have the lung cancer detected at a MCED screening at a stage (e.g., stage II) earlier than when the lung cancer would have been clinically diagnosed (e.g., stage IV). As another example, another participant with breast cancer may have the cancer detected at a MCED screening at the same stage the cancer would have been clinically diagnosed but at an earlier time within that stage.

As shown in FIG. 31, in one embodiment, there are five different subpopulations for a given type of cancer—(1) the first subpopulation are participants who are first detectable by MCED screenings from stage I of the cancer—thus, depending on the sensitivity of a MCED screen, their cancer may potentially be intercepted at stages I, II, III, or IV; (2) the second subpopulation are participants who are first detectable from stage II of the cancer, thus potentially intercepted at stages II, III, or IV; (3) the third subpopulation are participants who are first detectable from stage III of the cancer, thus potentially intercepted at stages III or IV; (4) the fourth subpopulation are participants who are first detectable from stage IV of the cancer, thus potentially intercepted at stage IV; and (5) the fifth subpopulation are participants who are not detectable by MCED screenings at any stage.

In one embodiment, the integration system can obtain the inferred number of cases for each subpopulation through, for example, observed case-control sensitivity studies. In one instance, a separate interception model is generated per cancer type, and the inferred values may be combined across different cancer types. In one instance, the integration system may obtain the inferred data from another source or may infer the data itself using empirical data from medical literature or publications, hospitals, and the like. An observation from FIG. 31 is if detectable stages in each subpopulation have similar or same dwell times, each subpopulation has different distributions of the duration of time over which the cancers can be intercepted. For example, a participant in the first subpopulation may have a longer duration over which the cancer could be intercepted by MCED screenings than a participant in the fourth subpopulation, since cancers in the first subpopulation may potentially be intercepted at all stages I, II, III, or IV. Thus, as described in further detail below, the integration system can integrate over future time to determine various metrics for MCED screenings as a function of age or time.

Throughout the remainder of the specification, the integration system is described using example detectability performances obtained from MCED screenings on CCGA2 studies, which are similar to CCGA3 studies described in section VI.C of the specification above. The cancer incidence information is obtained from SEER data and includes incidence for each 1-year of age for all cancers. The duration of detectability time may be approximated as a gamma distribution with mean and variance.

### VII.B. Integrating Over Time to Determine Interceptions Due to MCED Screenings and Effect on Mortality Rates

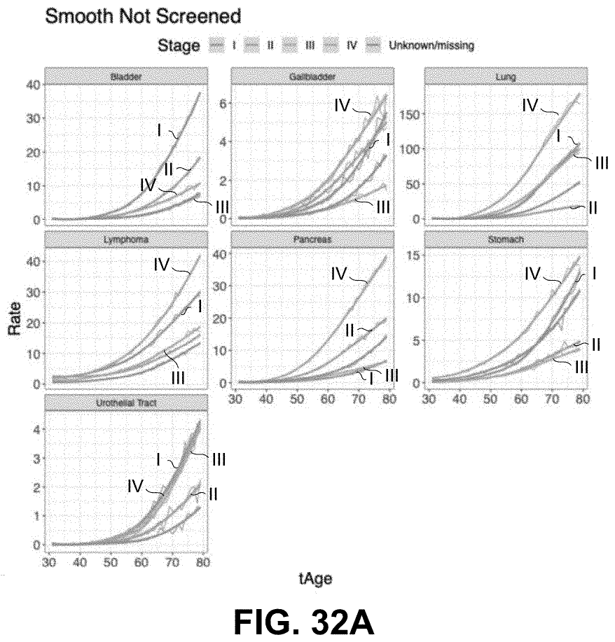

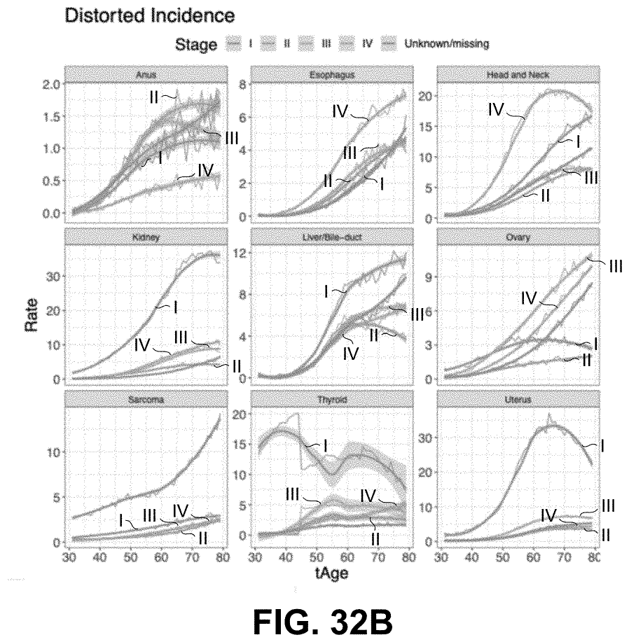

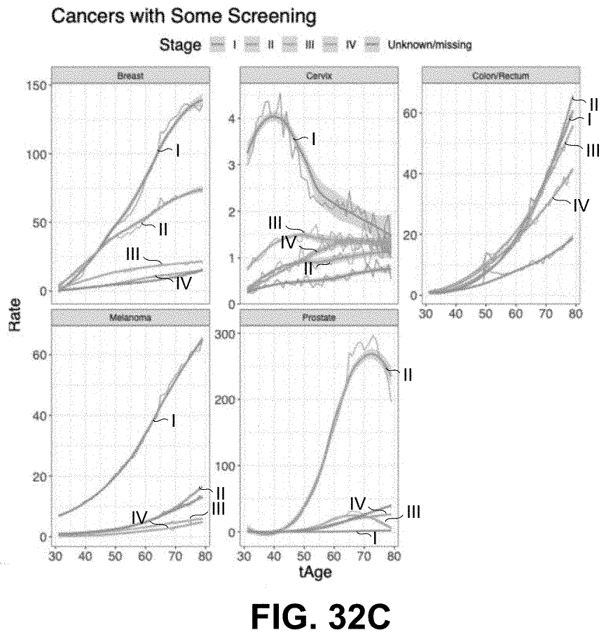

FIGS. 32A-32C illustrate cancer incidence as a function of age as obtained from SEER data, according to one embodiment. In one embodiment, the integration system obtains cancer incidence data per stage for a particular cancer type. FIG. 32A illustrates cancer incidence data for bladder, gallbladder, lung, lymphoma, pancreas, stomach, and urothelial tract cancer from SEER data. As shown in FIG. 32A, the cancer incidence data illustrates the rate of cancer per population of 100,000 at risk (vertical axis) as a function of age (horizontal axis). In general, there is an increase in cancer incidence with age (approximately 7-9% per year). Moreover, for the examples shown in FIG. 32A, there is a relatively smooth increase with age.

FIG. 32B illustrates cancer incidence data for anus, esophagus, head and neck, kidney, liver/bile-duct, ovary, sarcoma, thyroid, and uterus cancer from SEER data. As shown in FIG. 32B, certain cancer types vary by age differently from those shown in FIG. 32A. These variations may be due to, for example, diagnostic bias since thyroid cancer is redefined at age 45 for individuals and liver/bile-duct cancer is not examined in individuals likely to die. These variations may also be due to cohort effects such as exposure to risk factors, age effects such as hormone exposures, and surgical history such as increased uterus removal by older age.

FIG. 32C illustrates cancer incidence data for breast, cervix, colon/rectum, melanoma, and prostate cancer, which are cancers with some amount of ordinary care screening. As shown in FIG. 32C, for breast cancer, stage distribution shifts significantly when mammography screenings start (approximately age 50). For colon/rectum cancer, there are sudden increases in cancer incidence around ages 50, 65 due to diagnostic intensity changes. For prostate cancer, the variations may be due to prostate-specific antigen (PSA) diagnostic intensities, especially for stage II incidence.

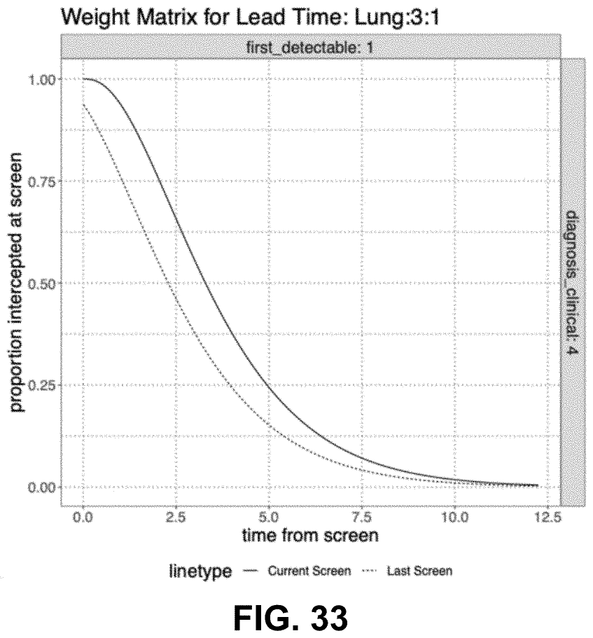

FIG. 33 illustrates a weight matrix for a MCED screening as a function of time from screen for a subpopulation that would be clinically diagnosed at stage IV but is first detectable at stage I for lung cancer, according to one embodiment. In one embodiment, the integration system generates one or more weight matrices. A weight matrix may correspond to a respective subpopulation of participants that were determined to be clinically diagnosed at a particular stage but were first detectable through MCED screenings at an earlier or the same stage but at an earlier time. Thus, the weight matrices may correspond to one or more subpopulations that are determined to be clinically diagnosed at stage I but were first detectable at stage I, determined to be clinically diagnosed at stage II but were first detectable at either stages I or II, determined to be clinically diagnosed at stage III but were first detectable at either stages I, II, or III, and determined to be clinically diagnosed at stage IV but were first detectable at either stages I, II, III, or IV.

In particular, the weight matrix can illustrate the proportion of participants intercepted at a given MCED screening (“current screen”) (vertical axis) as a function of the time of the clinical diagnosis from MCED screen. Thus, for a given MCED screening, a participant that would be clinically diagnosed in the future may be intercepted at the MCED screening if the detectable sojourn time of the cancer is greater than the time from screen of the clinical diagnosis. FIG. 33 in particular illustrates the weight matrix for a subpopulation that would be clinically diagnosed with lung cancer at stage IV but is first detectable at stage I. Thus, based on the solid curve in FIG. 33, there would approximately be a 75% likelihood that a participant who would be clinically diagnosed at 2.3 years from the MCED screen will be detected at the screening event (at time “0”).

In one instance, the current screen for a weight matrix may be a prevalence screen, and the proportion intercepted is given by the solid curve. In another instance, the current screen may be an incidence screen that comes after one or more previous screens. For example, one or more screening events of one-year intervals may be simulated, and the current screen may be the third MCED screen where the first screening event was two years before and the second screening event was one year before the current screen. In such an instance, the dotted curve illustrates the weight matrix for the previous screen that is adjusted according to the time of the current screen. The proportion intercepted for participants at each time from screen can be given by the difference between the solid curve and the dotted curve, as participants who were previously intercepted cannot be intercepted again. For example, the proportion of participants that would be clinically diagnosed from 5 years of the current screen would approximately be the difference between 25% (current screen) and 13% (last screen).

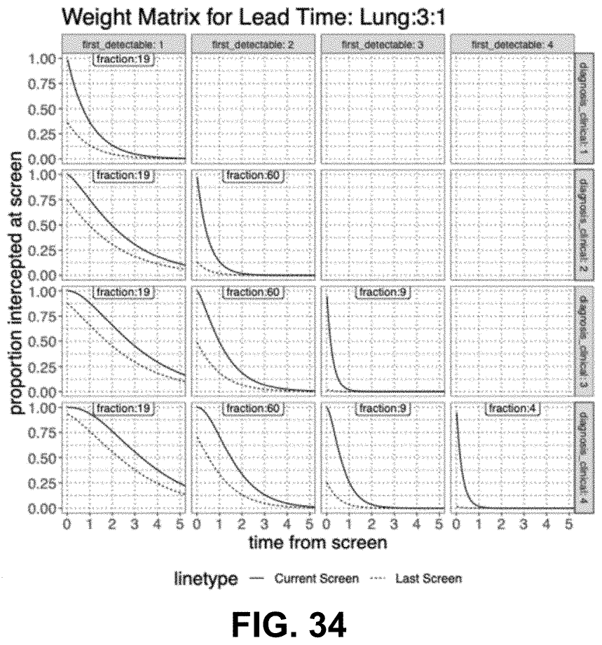

FIG. 34 illustrates weight matrices for a MCED screening as a function of time from screen for one or more subpopulations, according to one embodiment. Specifically, FIG. 34 illustrates weight matrices generated for each of the subpopulations described above in conjunction with FIG. 31. In one embodiment, the weight matrices are obtained from the interception model to obtain for each subpopulation, the proportion of participants that would be intercepted at a given screening as a function of time of clinical diagnosis from the screening event. For example, the weight matrix for the subpopulation that would be clinically diagnosed at stage IV and is first detectable at stage IV is relatively very steep because stage IV lasts a very short time for lung cancer. In other words, if a participant in the subpopulation would get clinically diagnosed one year or after, there is almost minimal chance that the participant is at stage IV at the time of MCED screening.

Thus, the weight matrix for a subpopulation can be the distribution of the probability of intercepting future incidences of cancer by lead time. The weight matrix thus may depend on dwell times at each stage of cancer but not on the cancer incidence or changes in cancer incidence over time. Moreover, while the weight matrix computes the fraction of future incidence that was intercepted, this may not be the same as the stage at diagnosis. Rather, the stage at interception may be a variable lead time which depends on the stage of future clinical diagnosis and MCED screening intervals. For example, if the screening intervals are relatively shorter, the stage at interception may generally be at an earlier stage, while if the screening intervals are relatively longer, the weight matrix for an incidence screen may approach the distribution for the prevalence screen. Thus, individuals that benefit the most from screenings can have the largest stage-shift and most delay in time-to-benefit for mortality.

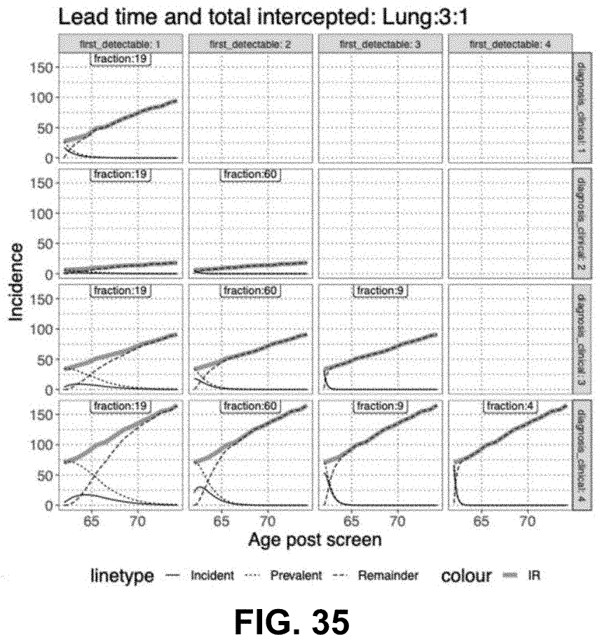

FIG. 35 illustrates intercepted incidences of lung cancer as a function of age post screen for a particular cancer type and starting age, according to one embodiment. In the example shown in FIG. 35, the intercepted incidences are plotted for a population with a starting age of 62 years. The curve with line type “IR” (in red) illustrates cancer incidence based on clinical diagnosis. Thus, each cancer incidence curve for a subpopulation that would be clinically diagnosed at stage I, stage II, stage III, and stage IV may respectively correspond to the stage I, stage II, stage III, and stage IV cancer incidence data illustrated in FIG. 32A for lung cancer. Note that the proportion of the population that is first detectable at stage I is 0.19, the proportion that is first detectable at stage II is 0.60, the proportion that is first detectable at stage III is 0.09, and the proportion that is first detectable at stage IV is 0.04.

The curve with the dotted line type “prevalent” illustrates the cumulated intercepted incidences for the current MCED screen and is generated by multiplying the cancer incidence starting from the age of the population at the current screen with the weight matrix for the current screen. For example, to generate the intercepted incidences from the future population of participants that would be of age 65 years old post the screening, the cancer incidence value at age 65 years old is multiplied with the value of the weight matrix corresponding to 3 years post screen, assuming a population age of 62 years old. Thus, this number represents the number of intercepted cases the MCED screening would detect and would “pull from” a future population of participants that would be 65 years of age in the future and would be fated to be clinically diagnosed at a particular stage of lung cancer (indicated by each row). This calculation can be performed for all values of the age post screen to generate the prevalent incidence curve.

The curve with the solid line type “incident” illustrates the intercepted incidences for the current screen not taking into account interceptions from one or more previous screens. The incident curve may be generated by multiplying the cancer incidence curve with the difference between the prevalence weight matrix and the incidence weight matrix. In addition, the curve with the dotted line type “remainder” illustrates the missed future incidences and represents the number of cases that were not detected by the current screen. Thus, the remainder curve may be the difference between the cancer incidence curve and the prevalent curve.

In one embodiment, the integration system can sum the intercepted incidences over the age post screen to determine the total number of intercepted incidences for a current MCED screen either using the prevalence curve or the incidence curve. For example, the integration system may sum over all intercepted incidences indicated in the prevalent curve to determine the total number of interceptions made at the current screen assuming the current screen is the prevalent screen. In other words, this value represents the number of cases that were intercepted or pulled from the future for a given population due to the current screen.

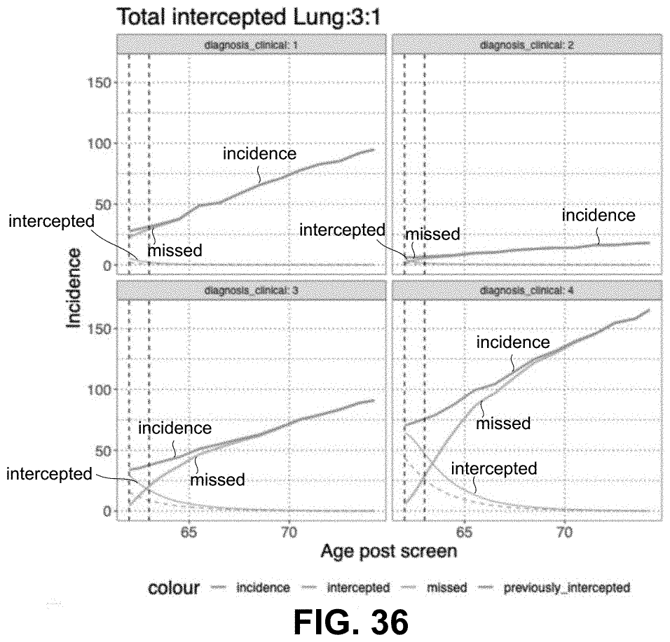

FIG. 36 illustrates intercepted incidences of lung cancer by clinical diagnosis stage, according to one embodiment. Specifically, the intercepted incidence curves generated in FIG. 36 are similar to FIG. 35 but are added up over all subpopulations per clinical diagnosis stage. The “incidence” curve illustrates the cancer incidence by original diagnosis. The “intercepted” curve illustrates the number of intercepted cases from the current screen assuming it is the first MCED screen performed for participants starting at the age of 62 years old. The “missed” curve illustrates the number of missed incidences by the current screen and the slope may represent how fast the pool of participants that are to be clinically diagnosed are refilled. The “previously intercepted” curve illustrates the number of intercepted incidences from a previous screen (when the participants were 61 years of age) assuming that the current screen is an incidence screen. In other words, FIG. 36 illustrates variable depletion of future incidence depending on stage of clinical diagnosis.

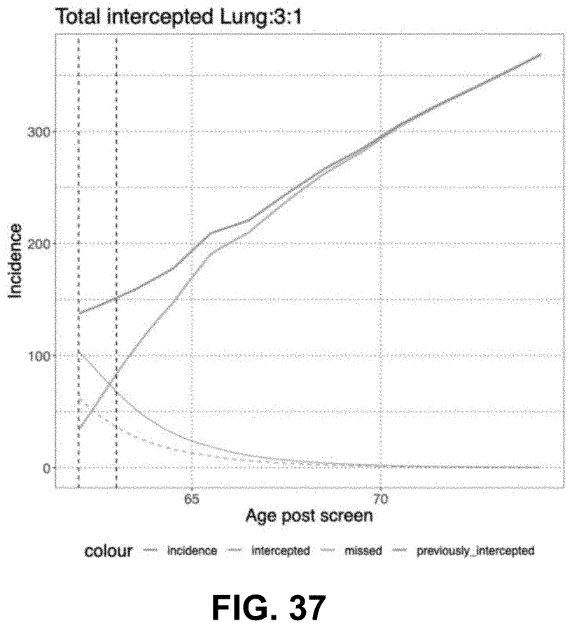

FIG. 37 illustrates intercepted incidences of lung cancer over all clinical diagnosis stages, according to one embodiment. In particular, the intercepted incidence curves generated in FIG. 37 are similar to FIG. 36 but are added up across all clinical diagnosis stages. As described above, the total number of intercepted cases for a given MCED screen can be determined by summing over all intercepted incidences indicated in the intercepted curve to determine the total number of interceptions made at the current screen. Based on the refresh rate of cancer incidence depleted by interception, the integration system can estimate an appropriate screening interval from the incidence curves.

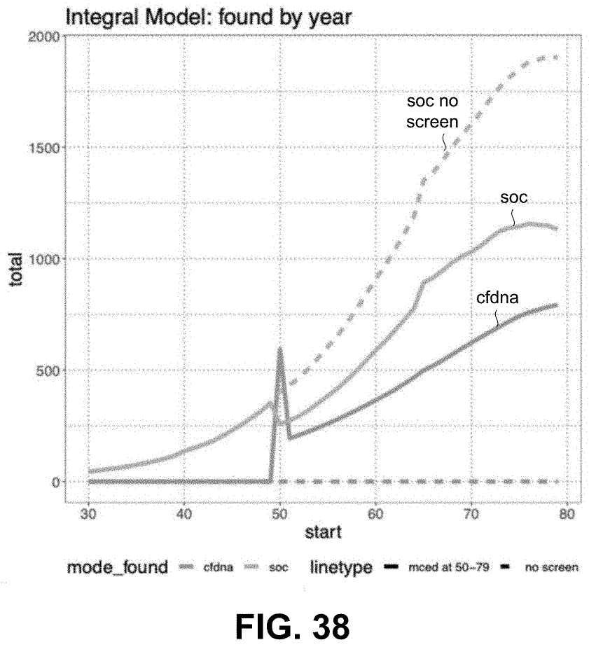

FIG. 38 illustrates intercepted incidences for each starting age, according to one embodiment. Specifically, FIG. 38 assumes that participants are screened through MCED at a starting age of 50 years old and have been screened in one-year intervals. For each start age, the integration system computes the effect of prevalence screening (at age 50) and the effect of subsequent incidence screenings by summing the intercepted incidences across age post screen, similarly to that described in conjunction with FIGS. 35-37. The curve with legend “soc no screen” indicates usual cancer incidence in the presence of no MCED screenings. The curve with legend “cfdna” illustrates the intercepted incidences at each start age, assuming the MCED screenings start at age 50. As shown in FIG. 38, there is a sudden spike of intercepted cases at age 50 because the MCED screenings are “turned on” at this age and the sum of the future incidences that are detected at screening is typically significantly more than the one-year usual cancer incidence. In some cases, the intercepted incidences that are determined by age can be similar to other types of models, such as the interception model described in section VII.A since cancer incidence does not change much over short lead time.

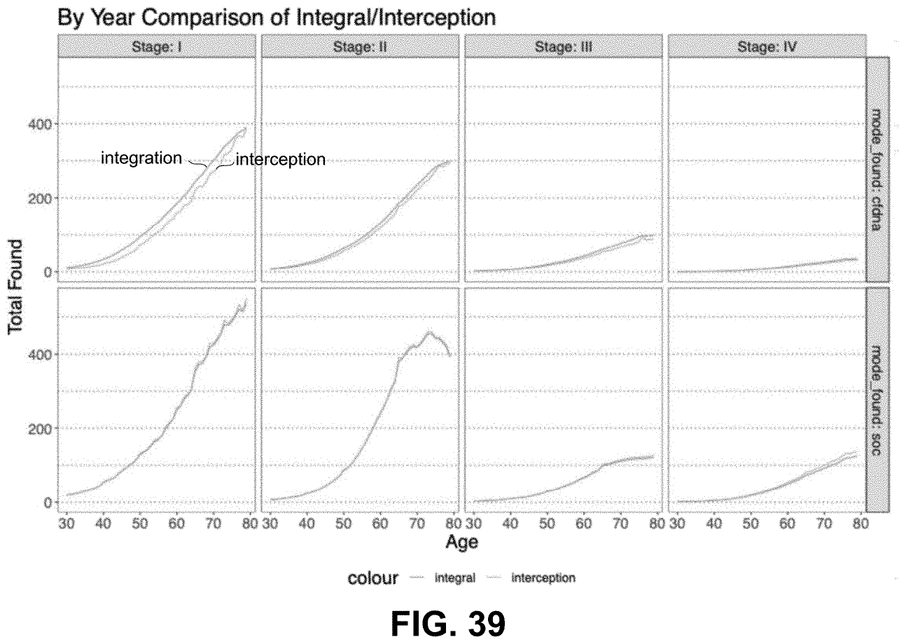

FIG. 39 illustrates a comparison of interception incidences modeled by the integration system with interception incidences modeled by an interception model, according to one embodiment. In the results of FIG. 39, an example model for simulating the number of interceptions, such as the interception model described in section VII.A, is used to generate the intercepted incidences with age, and these results are compared to those generated by the integration system. The top row illustrates the number of interceptions, and the bottom row illustrates the number of cancers found through usual clinical diagnosis. While the integration system incorporates changes of cancer incidence with age, the incidence generated by the interception model may be agnostic to how cancer incidence changes locally for a given age. However, since the integration system pulls incidences from the future (approximately 2-3 years from the future depending on dwell times for cancer) when there is generally more cancer incidence, the intercepted incidences for the integration system is relatively higher than that of the interception model.

As described above, the integration system integrates, over future time, to determine various metrics for MCED screenings as a function of age or time without simulating individual trajectories of participants that result in high computational power. The integration system can provide insight into how varying cancer incidence over time would affect the interceptions from MCED screenings, as described in detail in FIGS. 35-39. The integration system described herein provides a flexible way to incorporate various types of cancer incidence curves, even those that are non-smooth, which may not be possible or may be technically difficult to incorporate for other types of models. The analysis generated by the integration system can be provided to an analysis module (that is a part of the integration system or is separate from) such that the analysis module can determine various parameters of early detection screening, such as optimal screening intervals, for improving stage shift and mortality reduction of participants in an enrollment study.

### VII.C. Process of Integration System

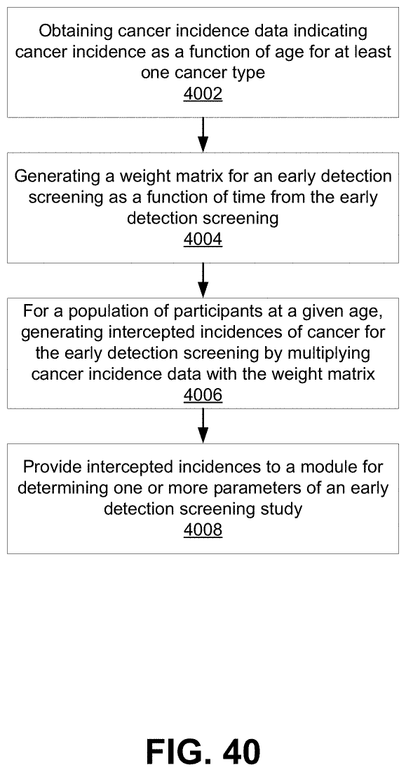

FIG. 40 illustrate a flowchart describing a process of an integrated method for determining intercepted incidences for MCED screenings, in accordance with an embodiment. One or more of the steps in FIG. 40 may be performed by the integration system but embodiments are not limited hereto.

The integration system obtains 4002 cancer incidence data indicating cancer incidence as a function of age for at least one cancer type. The cancer incidence data for an age may indicate a number of clinically diagnosed incidences of cancer at the age. The integration system generates 4004 a weight matrix for an early detection screening as a function of time from the early detection screening. For a set of participants that would be clinically diagnosed at a particular time from the early detection screening, the weight matrix may indicate a proportion of the set of participants that would be intercepted by the early detection screening. For a population of participants at a given age at a time of the early detection screening, the integration system generates 4006 intercepted incidences of cancer for the early detection screening by multiplying the cancer incidence data starting from the given age with the weight matrix for the early detection screening. The integration system provides 4008 the intercepted incidences to a module for determining one or more parameters of an early detection screening study.

### VIII. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method, comprising:
simulating, using a simulation model, a cancer progression for each of a plurality of participants, wherein a first subset of the participants are assigned to a control group and a second subset of the participants are assigned to a intervention group;
for each simulated participant in the plurality of participants:
sampling a cancer type and a diagnostic stage of cancer for the participant, wherein the participant is expected to be clinically diagnosed with the cancer type at the diagnostic stage after a diagnosis time period for the participant has passed with respect to an enrollment time;
generating a cancer trajectory for the participant that indicates progression of one or more stages of cancer for the participant and a survival time based on the cancer type and the diagnostic stage for the participant; and
determining a first detection time that indicates a time when the cancer of the participant was correctly detected at a first detection stage equal to or earlier than the diagnostic stage;
for each simulated participant in the second subset of participants of the intervention group, simulating early cancer detection screening by one or more machine-learned cancer detection models for at least one screening time, by:
generating an indication for the participant that indicates whether the machine-learned cancer detection models predict that the participant has cancer;
if the indication of the cancer detection models predict the participant has cancer and the participant has an earlier stage of cancer at the at least one screening time than the first detection stage, generating a second detection stage as the earlier stage and updating the survival time based on the second detection stage; and
for each of the first subset of participants and the second subset of participants, evaluating one or more metrics related to progression of cancer; and
evaluating a difference between the one or more metrics of the first subset of participants and the second subset of participants to determine an effect of early cancer detection screening.

2. The method of claim 1, wherein the steps of claim 1 are performed on two or more cores of a hardware processing system in parallel, and wherein the steps of claim 1 are implemented using the Julia programming language.

3. The method of claim 1, wherein the cancer type for the participant is sampled from a first multinomial distribution characterized by a set of cancer types and corresponding probabilities of the set of cancer types, and wherein the diagnostic stage for the participant is sampled from a second multinomial distribution characterized by a set of stages and corresponding probabilities of the set of stages.

4. (canceled)

5. The method of claim 1, further comprising:
obtaining cancer incidence data as a function of age and generating a cumulative distribution function (CDF) of cancer incidence from the cancer incidence data;
for each participant in the plurality of participants:
assigning an age to the participant;
sampling a value from a uniform distribution starting from a quantile that corresponds to the age of the participant; and
applying an inverse of the CDF of the cancer incidence to the sampled value to generate the diagnosis time period for the participant.

6. The method of claim 1, wherein generating a cancer trajectory for the participant further comprises:
sampling one or more dwell times for one or more stages of the participant's cancer, each dwell time indicating a time span the cancer will stay in a respective stage before progressing to a next stage; and
determining start times of the one or more stages based on the sampled dwell times.

7. The method of claim 1, wherein the one or more machine-learned cancer detection models include a machine-learned model configured to output predictions on presence of cancer for multiple types of cancer.

8. The method of claim 1, wherein the at least one screening time includes a plurality of screening times, and simulating the early cancer detection screening further comprises:
generating a plurality of indications for the participant, each indication indicating whether the machine-learned cancer detection models predict the participant has cancer at a respective screening time in the plurality of screening times; and
if two or more indications for two or more screening times detect the participant has cancer and the participant has cancer at the two or more screening times, selecting an earlier one of the two or more screening times; and
if the participant has the earlier stage of cancer at the selected screening time than the first detection stage, classifying the second detection stage as the earlier stage.

9. (canceled)

10. The method of claim 1, wherein generating the indication comprises:
determining whether the participant has cancer at the at least one screening time;
if the participant has cancer at the at least one screening time, sampling the indication based on a sensitivity of the one or more machine-learned cancer detection models; and
if the participant does not have cancer at the at least one screening time, sampling the indication based on a specificity of the one or more machine-learned cancer detection models.

11. The method of claim 1, wherein simulating the early cancer detection screening further comprises if the indication of the cancer detection models do not predict the participant has cancer or the participant has a later stage of cancer at the at least one screening time than the first detection stage, determining the first detection stage as the second detection stage, and wherein the one or more metrics include, for each of the control group and the intervention group, statistics related to number of cancers detected at stages I or II of progression, number of cancers detected at stages III or IV of progression, and mortality rate.

12. (canceled)

13. A system, comprising:
a processor;
a computer readable storage medium for storing instructions executable by a processor, the instructions comprising:
simulating, using a simulation model, a cancer progression for each of a plurality of participants, wherein a first subset of the participants are assigned to a control group and a second subset of the participants are assigned to a intervention group;
for each simulated participant in the plurality of participants:
sampling a cancer type and a diagnostic stage of cancer for the participant, wherein the participant is expected to be clinically diagnosed with the cancer type at the diagnostic stage after a diagnosis time period for the participant has passed with respect to an enrollment time;
generating a cancer trajectory for the participant that indicates progression of one or more stages of cancer for the participant and a survival time based on the cancer type and the diagnostic stage for the participant; and
determining a first detection time that indicates a time when the cancer of the participant was correctly detected at a first detection stage equal to or earlier than the diagnostic stage;
for each simulated participant in the second subset of participants of the intervention group, simulating early cancer detection screening by one or more machine-learned cancer detection models for at least one screening time, by:
generating an indication for the participant that indicates whether the machine-learned cancer detection models predict that the participant has cancer;
if the indication of the cancer detection models predict the participant has cancer and the participant has an earlier stage of cancer at the at least one screening time than the first detection stage, generating a second detection stage as the earlier stage and updating the survival time based on the second detection stage; and
for each of the first subset of participants and the second subset of participants, evaluating one or more metrics related to progression of cancer; and
evaluating a difference between the one or more metrics of the first subset of participants and the second subset of participants to determine an effect of early cancer detection screening.

14. The system of claim 13, wherein the steps of claim 1 are performed on two or more cores of a hardware processing system in parallel, and wherein the steps of claim 1 are implemented using the Julia programming language.

15-24. (canceled)

25. A non-transitory computer readable storage medium for storing instructions executable by a processor, the instructions comprising:
simulating, using a simulation model, a cancer progression for each of a plurality of participants, wherein a first subset of the participants are assigned to a control group and a second subset of the participants are assigned to a intervention group;
for each simulated participant in the plurality of participants:
sampling a cancer type and a diagnostic stage of cancer for the participant, wherein the participant is expected to be clinically diagnosed with the cancer type at the diagnostic stage after a diagnosis time period for the participant has passed with respect to an enrollment time;
generating a cancer trajectory for the participant that indicates progression of one or more stages of cancer for the participant and a survival time based on the cancer type and the diagnostic stage for the participant; and
determining a first detection time that indicates a time when the cancer of the participant was correctly detected at a first detection stage equal to or earlier than the diagnostic stage;
for each simulated participant in the second subset of participants of the intervention group, simulating early cancer detection screening by one or more machine-learned cancer detection models for at least one screening time, by:
generating an indication for the participant that indicates whether the machine-learned cancer detection models predict that the participant has cancer;
if the indication of the cancer detection models predict the participant has cancer and the participant has an earlier stage of cancer at the at least one screening time than the first detection stage, generating a second detection stage as the earlier stage and updating the survival time based on the second detection stage; and
for each of the first subset of participants and the second subset of participants, evaluating one or more metrics related to progression of cancer; and
evaluating a difference between the one or more metrics of the first subset of participants and the second subset of participants to determine an effect of early cancer detection screening.

26. The non-transitory computer readable storage medium of claim 25, wherein the steps of claim 1 are performed on two or more cores of a hardware processing system in parallel, and wherein the steps of claim 1 are implemented using the Julia programming language.

27. The non-transitory computer readable storage medium of claim 25, wherein the cancer type for the participant is sampled from a first multinomial distribution characterized by a set of cancer types and corresponding probabilities of the set of cancer types, and wherein the diagnostic stage for the participant is sampled from a second multinomial distribution characterized by a set of stages and corresponding probabilities of the set of stages.

28. (canceled)

29. The non-transitory computer readable storage medium of claim 25, further comprising:
obtaining cancer incidence data as a function of age and generating a cumulative distribution function (CDF) of cancer incidence from the cancer incidence data;
for each participant in the plurality of participants:
assigning an age to the participant;
sampling a value from a uniform distribution starting from a quantile that corresponds to the age of the participant; and
applying an inverse of the CDF of the cancer incidence to the sampled value to generate the diagnosis time period for the participant.

30. The non-transitory computer readable storage medium of claim 25, wherein generating a cancer trajectory for the participant further comprises:
sampling one or more dwell times for one or more stages of the participant's cancer, each dwell time indicating a time span the cancer will stay in a respective stage before progressing to a next stage; and
determining start times of the one or more stages based on the sampled dwell times.

31. The non-transitory computer readable storage medium of claim 25, wherein the one or more machine-learned cancer detection models include a machine-learned model configured to output predictions on presence of cancer for multiple types of cancer.

32. The non-transitory computer readable storage medium of claim 25, wherein the at least one screening time includes a plurality of screening times, and simulating the early cancer detection screening further comprises:
generating a plurality of indications for the participant, each indication indicating whether the machine-learned cancer detection models predict the participant has cancer at a respective screening time in the plurality of screening times; and
if two or more indications for two or more screening times detect the participant has cancer and the participant has cancer at the two or more screening times, selecting an earlier one of the two or more screening times; and
if the participant has the earlier stage of cancer at the selected screening time than the first detection stage, classifying the second detection stage as the earlier stage.

33. (canceled)

34. The non-transitory computer readable storage medium of claim 25, wherein generating the indication comprises:
determining whether the participant has cancer at the at least one screening time;
if the participant has cancer at the at least one screening time, sampling the indication based on a sensitivity of the one or more machine-learned cancer detection models; and
if the participant does not have cancer at the at least one screening time, sampling the indication based on a specificity of the one or more machine-learned cancer detection models.

35. The non-transitory computer readable storage medium of claim 25, wherein simulating the early cancer detection screening further comprises if the indication of the cancer detection models do not predict the participant has cancer or the participant has a later stage of cancer at the at least one screening time than the first detection stage, determining the first detection stage as the second detection stage, wherein the one or more metrics include, for each of the control group and the intervention group, statistics related to number of cancers detected at stages I or II of progression, number of cancers detected at stages III or IV of progression, and mortality rate.

36-37. (canceled)
